(19) United States
(12) Patent Application Publication
(10) Pub. No.: US 2022/0347117 A1
(43) Pub. Date: Nov. 3, 2022

(54) DOSAGE FORMS HAVING EQUIVALENT BIOCOMPARABLE PROFILES

(71) Applicant: Aquestive Therapeutics, Inc., Warren, NJ (US)

(72) Inventors: Alexander Mark Schobel, Vero Beach, FL (US); Stephen Wargacki, Pittstown, NJ (US)

(21) Appl. No.: 17/691,107

(22) Filed: Mar. 9, 2022

Related U.S. Application Data
(60) Provisional application No. 63/158,400, filed on Mar. 9, 2021.

(51) Int. Cl. A61K 9/70; A61K 9/00; A61K 45/06; A61K 47/02; A61K 47/10

(52) U.S. Cl. CPC A61K 9/7007; A61K 9/006; A61K 45/06; A61K 47/02; A61K 47/10

(57) ABSTRACT

Self-supporting film unit dosage forms for oral transmucosal delivery are disclosed herein containing an active. These film dosage forms for oral transmucosal delivery have a substantially equivalent pharmacokinetic profile to dosage forms administered by another route or in another dosage form (e.g., tablet, patch) and yet have a different amount of active contained therein.

### CROSS-REFERENCE TO RELATED APPLICATIONS

This application claims priority to, and the benefit of U.S. Application No. 63/158,400, filed Mar. 9, 2021, the entire contents of which are incorporated by reference herein.

### FIELD OF THE DISCLOSURE

The present disclosure relates to dosage forms, such as self-supporting films, for delivery of one or more pharmaceutical actives via the mucosa, such as the oral mucosa, that provide a substantially equivalent pharmacokinetic profile to that of individual dosage forms of the same active(s) having a different route of administration.

### BACKGROUND

Mucosal membranes, such as the oral mucosa, are a convenient route for delivering drugs to the body due to the fact that they are highly vascularized and permeable, providing increased bioavailability and rapid onset of action because it does not pass through the digestive system and thereby avoids first pass metabolism. In particular, the buccal and sublingual tissues offer advantageous sites for drug delivery because they are highly permeable regions of the oral mucosa, allowing drugs diffusing from the oral mucosa to have direct access to systemic circulation. This also offers increased convenience and therefore increased compliance in patients.

There are many reasons why the oral mucosa might be an attractive site for the delivery of therapeutic agents into the systemic circulation. Due to the direct drainage of blood from the buccal epithelium into the internal jugular vein first-pass metabolism in the liver and intestine may be avoided. First-pass effect can be a major reason for the poor bioavailability of some compounds when administered orally. Additionally, the mucosa lining the oral cavity is easily accessible, which ensures that a dosage form can be applied to the required site and can be removed easily in the case of an emergency. However, like the skin, the buccal mucosa acts as a barrier to the absorption of xenobiotics, which can hinder the permeation of compounds across this tissue. Consequently, the identification of safe and effective penetration enhancers has become a major goal in the quest to improve oral mucosal drug delivery.

The permeability of the buccal mucosa is greater than that of the skin, but less than that of the intestine. The differences in permeability are the result of structural differences between each of the tissues. The absence of organized lipid lamellae in the intercellular spaces of the buccal mucosa results in greater permeability of exogenous compounds, compared to keratinized epithelia of the skin; while the increased thickness and lack of tight junctions results in the buccal mucosa being less permeable than intestinal tissue.

The existence of hydrophilic and lipophilic regions in the oral mucosa has led researchers to postulate the existence of two routes of drug transport through the buccal mucosa—paracellular (between the cells) and transcellular (across the cells).

The buccal mucosa delineates the inside lining of the cheek as well as the area between the gums and upper and lower lips and it has an average surface area of 100 cm². Structurally the sublingual mucosa is comparable to the buccal mucosa but is thinner. Blood flow to the sublingual mucosal is slower compared with the buccal mucosa and is of the order of 1.0 ml/min-1/cm-2.

A pharmaceutical composition can be designed to deliver a pharmaceutical active in a deliberate and tailored way. However, solubility and permeability of the pharmaceutical active in vivo, in particular, in the mouth of a subject, can vary tremendously. A particular class of permeation enhancer can improve the uptake and bioavailability of the pharmaceutical active in vivo. Thus, for certain drugs, or pharmaceutical actives, a permeation enhancer can help to overcome the mucosal barrier and improve permeability. Permeation enhancers reversibly modulate the penetrability of the barrier layer in favor of drug absorption. Permeation enhancers facilitate transport of molecules through the epithelium. Absorption profiles and their rates can be controlled and modulated by a variety of parameters, such as but not limited to film size, drug loading, enhancer type/loading, polymer matrix release rate and mucosal residence time.

Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery—Current status and future prospects, Intl Journal of Pharmaceutics, 47(2014) 498-506, which is incorporated by reference herein. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium, as well as the emergence of new analytical techniques to study the oral cavity, and the progressing development of in silico models predictive of buccal and sublingual permeation, prospects are encouraging. For example, WO 2012/053006 discloses oral fast dissolving films comprising polymers and a therapeutically effective amount of ondanetron/tadalafil are bioequivalent to conventional immediate release dosage forms containing the same amount of the actives. As stated therein, an advantage of oral films is bypass of the gastrointestinal (GI) tract and barriers in the GI tract to drug absorption such as first pass metabolism and decomposition of the active in the stomach.

In order to deliver broader classes of drugs across the buccal mucosa, reversible methods of reducing the barrier potential of this tissue should be employed. This requisite has fostered the study of penetration enhancers that will safely alter the permeability restrictions of the buccal mucosa. It has been shown that buccal penetration can be improved by using various classes of transmucosal and transdermal penetration enhancers such as bile salts, surfactants, fatty acids and their derivatives, chelators, cyclodextrins and chitosan.

There is a need for improved oral film dosage forms that provide the desired pharmacokinetic profiles for the active(s) contained therein, while providing an adhesive effect in the mouth.

### SUMMARY OF THE DISCLOSURE

A dosage form for mucosal delivery of an active is disclosed herein that comprises an active and a carrier for the active. When the dosage form is administered to the mucosa of a mammal, a pharmacokinetic profile of the active is substantially equivalent to the pharmacokinetic profile of a corresponding enterally delivered dosage form containing the active, and the dosage form for mucosal delivery contains about 0.5% to about 75% less active per individual dosage unit than the corresponding enterally delivered dosage form.

A self-supporting film dosage form for mucosal delivery of a pharmaceutical active is disclosed herein that comprises a water-soluble or water swellable film-forming polymer matrix containing the pharmaceutical active. When the film dosage form is administered to the mucosa of a mammal, a pharmacokinetic profile of the pharmaceutical active is substantially equivalent to the pharmacokinetic profile of a corresponding enterally delivered dosage form containing the pharmaceutical active, and the film dosage form for mucosal delivery contains about 0.5% to about 75% less active per individual dosage unit than the corresponding enterally delivered dosage form.

A self-supporting film dosage form for oral mucosal delivery of a pharmaceutical active is disclosed herein that comprises a water-soluble or water swellable film-forming polymer matrix containing the pharmaceutical active. When the film dosage form is administered to the oral mucosa of a mammal, a pharmacokinetic profile of the pharmaceutical active is substantially equivalent to the pharmacokinetic profile of a corresponding enterally delivered dosage form containing the pharmaceutical active, and the film dosage form for mucosal delivery contains about 0.5% to about 75% less active per individual dosage unit than the corresponding enterally delivered dosage form.

A pharmaceutically acceptable dosage form for mucosal delivery of an active comprising an active, a polyethylene-oxide composition, and about 0.02% to about 3% by weight of silicon dioxide is disclosed herein. The dosage form for mucosal delivery, which may comprise water or a water-containing solvent, is an individual unit dosage form, each individual unit dosage form contains about 0.5% to about 50% less active than the corresponding enterally delivered dosage form, and, when it is administered to a patient, one or more of the AUC, Cmax or Tmax of the active is about 80% to about 125% of the AUC, Cmax or Tmax, respectively, of the corresponding enterally delivered dosage form.

A pharmaceutically acceptable film for mucosal delivery of a pharmaceutical active comprising a pharmaceutical active and a water-soluble or water swellable film-forming polymer matrix is disclosed herein. The polymer matrix includes a polyethylene oxide composition, water or a water-containing solvent, and about 0.02% to about 3% by weight of silicon dioxide. The film is an individual unit dosage form, each individual unit dosage form contains about 0.5% to about 50% less active than the corresponding enterally delivered dosage form, and, when it is administered to a patient, one or more of the AUC, Cmax or Tmax of the pharmaceutical active is about 80% to about 125% of the AUC, Cmax or Tmax, respectively, of the corresponding enterally delivered dosage form.

Disclosed herein is a pharmaceutically acceptable film for oral mucosal delivery of a pharmaceutical active comprising a pharmaceutical active and a water-soluble or water swellable film-forming polymer matrix. The polymer matrix includes a polyethylene oxide composition, water or a water-containing solvent, and about 0.02% to about 3% by weight of silicon dioxide. The oral film is an individual unit dosage form, each individual unit dosage form contains about 0.5% to about 50% less active than the corresponding enterally delivered dosage form, and, when it is administered to a patient, one or more of the AUC, Cmax or Tmax of the pharmaceutical active is about 80% to about 125% of the AUC, Cmax or Tmax, respectively, of the corresponding enterally delivered dosage form.

A dosage form for mucosal delivery of a biological macromolecule active that comprises a carrier containing the active is disclosed herein. When the dosage form is administered to the mucosa of a mammal, a pharmacokinetic profile of the biological macromolecule active is substantially equivalent to the pharmacokinetic profile of a corresponding intravenously delivered dosage form containing the active, and the dosage form for mucosal delivery contains about 10% to about 10,000% more active per individual dosage unit than the corresponding intravenously delivered dosage form.

A self-supporting film dosage form for mucosal delivery of a biological macromolecule active that comprises a water-soluble or water swellable film-forming polymer matrix containing the active is disclosed herein. When the film dosage form is administered to the mucosa of a mammal, a pharmacokinetic profile of the active is substantially equivalent to the pharmacokinetic profile of a corresponding intravenously delivered dosage form containing the pharmaceutical active, and the film dosage form for mucosal delivery contains about 10% to about 10,000% more active per individual dosage unit than the corresponding intravenously delivered dosage form.

Also disclosed is a self-supporting film dosage form for oral mucosal delivery of a biological macromolecule active is that comprises a water-soluble or water swellable film-forming polymer matrix containing the active. When the film dosage form is administered to the oral mucosa of a mammal, a pharmacokinetic profile of the active is substantially equivalent to the pharmacokinetic profile of a corresponding intravenously delivered dosage form containing the pharmaceutical active, and the film dosage form for mucosal delivery contains about 10% to about 10,000% more active per individual dosage unit than the corresponding intravenously delivered dosage form.

Disclosed herein is a pharmaceutically acceptable film for mucosal delivery of a biological macromolecule active comprising the active and a water-soluble or water swellable film-forming polymer matrix is disclosed herein. The polymer matrix includes a polyethylene oxide composition, water or a water-containing solvent, and about 0.02% to about 3% by weight of silicon dioxide. The film is an individual unit dosage form, each individual unit dosage form contains about 10% to about 10,000% more active than the corresponding intravenously delivered dosage form, and, when it is administered to a patient, one or more of the AUC, Cmax or Tmax of the pharmaceutical active is about 80% to about 125% of the AUC, Cmax or Tmax, respectively, of the corresponding intravenously delivered dosage form.

Disclosed herein is a pharmaceutically acceptable film for oral mucosal delivery of a biological macromolecule active comprising the active and a water-soluble or water swellable film-forming polymer matrix. The polymer matrix includes a polyethylene oxide composition, water or a water-containing solvent, and about 0.02% to about 3% by weight of silicon dioxide. The oral film is an individual unit dosage form, each individual unit dosage form contains about 10% to about 10,000% more active than the corresponding intravenously delivered dosage form, and, when it is administered to a patient, one or more of the AUC, Cmax or Tmax of the pharmaceutical active is about 80% to about 125% of the AUC, Cmax or Tmax, respectively, of the corresponding intravenously delivered dosage form.

Disclosed herein is a method of treating a patient comprising administering a dosage form for mucosal delivery of an active that comprises a carrier containing an active, wherein a pharmacokinetic profile of the active is substantially equivalent to the pharmacokinetic profile of a corresponding enterally delivered dosage form containing the active, and the dosage form for mucosal delivery contains about 0.5% to about 75% less active per individual dosage unit than the corresponding enterally delivered dosage form, and a method of treating a patient comprising administering a dosage form for mucosal delivery of a biological macromolecule active that comprises a carrier containing the active, wherein a pharmacokinetic profile of the active is substantially equivalent to the pharmacokinetic profile of a corresponding intravenously delivered dosage form containing the active, and the dosage form for mucosal delivery contains about 10% to about 10,000% more active per individual dosage unit than the corresponding intravenously delivered dosage form.

Disclosed herein is a method of treating a patient comprising administering a self-supporting film dosage form for mucosal delivery of a pharmaceutical active that comprises a water-soluble or water swellable film-forming polymer matrix containing a pharmaceutical active, wherein a pharmacokinetic profile of the pharmaceutical active is substantially equivalent to the pharmacokinetic profile of a corresponding enterally delivered dosage form containing the pharmaceutical active, and the film dosage form for mucosal delivery contains about 0.5% to about 75% less active per individual dosage unit than the corresponding enterally delivered dosage form, and a method of treating a patient comprising administering a self-supporting film dosage form for mucosal delivery of a biological macromolecule active that comprises a water-soluble or water swellable film-forming polymer matrix containing the active, wherein a pharmacokinetic profile of the active is substantially equivalent to the pharmacokinetic profile of a corresponding intravenously delivered dosage form containing the active, and the film dosage form for mucosal delivery contains about 10% to about 10,000% more active per individual dosage unit than the corresponding intravenously delivered dosage form.

Disclosed herein is a method of treating a patient comprising administering a self-supporting film dosage form for oral mucosal delivery of a pharmaceutical active that comprises a water-soluble or water swellable film-forming polymer matrix containing a pharmaceutical active, wherein a pharmacokinetic profile of the pharmaceutical active is substantially equivalent to the pharmacokinetic profile of a corresponding enterally delivered dosage form containing the pharmaceutical active, and the film dosage form for mucosal delivery contains about 0.5% to about 75% less active per individual dosage unit than the corresponding enterally delivered dosage form, and a method of treating a patient comprising administering a self-supporting film dosage form for oral mucosal delivery of a biological macromolecule active that comprises a water-soluble or water swellable film-forming polymer matrix containing the active, wherein a pharmacokinetic profile of the active is substantially equivalent to the pharmacokinetic profile of a corresponding intravenously delivered dosage form containing the active, and the film dosage form for mucosal delivery contains about 10% to about 10,000% more active per individual dosage unit than the corresponding intravenously delivered dosage form.

Disclosed herein is a dosage form for mucosal delivery of an active that comprises a carrier containing the active, wherein, when the dosage form is administered to the mucosa of a mammal, a pharmacokinetic profile of the active is substantially equivalent to the pharmacokinetic profile of a corresponding enterally or parenterally delivered dosage form containing the active, and the dosage form for mucosal delivery contains about 0.5% to about 75% less active per individual dosage unit than the corresponding enterally or parenterally delivered dosage form, and methods of treating a patient by administering the same.

Disclosed herein is a self-supporting film dosage form for mucosal delivery of a pharmaceutical active that comprises a water-soluble or water swellable film-forming polymer matrix containing the pharmaceutical active, wherein, when the film dosage form is administered to the mucosa of a mammal, a pharmacokinetic profile of the pharmaceutical active is substantially equivalent to the pharmacokinetic profile of a corresponding enterally or parenterally delivered non-film dosage form containing the pharmaceutical active, and the film dosage form for mucosal delivery contains about 0.5% to about 75% less active per individual dosage unit than the corresponding enterally or parenterally delivered dosage form, and methods of treating a patient by administering the same.

Disclosed herein is a self-supporting film dosage form for oral mucosal delivery of a pharmaceutical active that comprises a water-soluble or water swellable film-forming polymer matrix containing the pharmaceutical active, wherein, when the film dosage form is administered to the oral mucosa of a mammal, a pharmacokinetic profile of the pharmaceutical active is substantially equivalent to the pharmacokinetic profile of a corresponding enterally or parenterally delivered non-film dosage form containing the pharmaceutical active, and the film dosage form for mucosal delivery contains about 0.5% to about 75% less active per individual dosage unit than the corresponding enterally or parenterally delivered dosage form, and methods of treating a patient by administering the same.

### BRIEF DESCRIPTION OF THE FIGURES

FIG. 1 depicts a self-supporting individual film dosage form of the disclosure.

FIG. 2 depicts how a mucosally delivered film dosage form of the disclosure, i.e., a 20 mg Diazepam film, exhibited improved bioavailability and Cmax relative to a 20 mg Diastat® rectal gel.

FIG. 3 depicts how a mucosally delivered film dosage form of the disclosure, i.e., a 15 mg Diazepam buccal film, exhibited bioavailability substantially equivalent to that of a 20 mg Diastat® rectal gel (20 mg diazepam rectal gel) in subjects in a fasted state but exhibited bioavailability that was significantly less in subjects in a fed state.

FIG. 4 depicts how a film dosage form of the disclosure, i.e., a 24 mg Dipivefrin film, exhibited enhanced bioavailability and Cmax when administered via transmucosal delivery versus oral absorption (film dosage form administered by swallowing).

FIG. 5A depicts the plasma concentrations of an active, i.e., Octreotide, in subjects following administration of a dosage form of the disclosure, i.e., an Octreotide 10 mg film, to the subjects by sublingual administration.

FIG. 5B depicts the plasma concentrations of an active, i.e., Octreotide, in subjects following administration of a dosage form of the disclosure, i.e., an Octreotide 10 mg film, to the subjects by enteral application.

### DETAILED DESCRIPTION OF THE DISCLOSURE

The disclosure includes a self-supporting film dosage form for oral mucosal delivery of a pharmaceutical active in individual dosage units comprising a water-soluble or water swellable film-forming polymer matrix containing a pharmaceutical active, wherein when the film dosage form is administered to the oral mucosa of a mammal, a pharmacokinetic profile of the pharmaceutical active is substantially equivalent to the pharmacokinetic profile of a corresponding enterally delivered dosage form containing the pharmaceutical active, and wherein the film dosage form for mucosal delivery contains about 0.5% to about 75% less active per individual dosage unit than the corresponding enterally delivered dosage form. Such a self-supporting film dosage form may have any of the following features: one or more of the AUC and Cmax of the film dosage form is about 80% to about 125% at a confidence level of at least about 80%, of the AUC and/or Cmax of the corresponding enterally delivered dosage form; the pharmacokinetic profile of the film dosage form (pKP_{film}) is about 90% to about 115% of the pharmacokinetic profile of the corresponding enterally delivered dosage form (pKP_{ingestible}) at a confidence level of at least about 70%; the pharmacokinetic profile of the film dosage form (pKP_{film}) is about 95% to about 110% of the pharmacokinetic profile of the corresponding enterally delivered dosage form (pKP_{ingestible}) at a confidence level of at least about 60%; the Tmax is about 80% to about 125% of the corresponding enterally delivered dosage form.

The disclosure includes a pharmaceutically acceptable film for oral mucosal delivery of a pharmaceutical active including: a. pharmaceutical active; and b. a water-soluble or water swellable film-forming polymer matrix including: i. a polyethylene oxide composition, ii. water or a water-containing solvent, iii. and silicon dioxide; wherein the oral film is an individual unit dosage form, wherein, when the individual unit dosage form is administered to the oral mucosa of a patient, one or more of the AUC and Cmax of the pharmaceutical active is about 80% to about 125% at a confidence level of at least about 80% of the AUC and/or Cmax of the corresponding enterally delivered dosage form, and wherein each individual unit dosage form contains about 0.5% to about 50% less active than the corresponding enterally delivered dosage form.

The disclosure includes a self-supporting film dosage form for oral mucosal delivery of a biological macromolecule active in individual dosage units including a water-soluble or water swellable film-forming polymer matrix containing the biological macromolecule active; wherein, when the film dosage form is administered to the oral mucosa of a patient, a pharmacokinetic profile of the biological macromolecule active is substantially equivalent to the pharmacokinetic profile of an intravenously delivered dosage form (pKP_{intravenously delivered}) of the biological macromolecule active, and wherein the film dosage form for oral mucosal delivery contains about 10 times to about 100,000 times more active per unit dose than the corresponding intravenously delivered dosage form. Such a self-supporting film dosage form may have any of the following features: wherein the pharmacokinetic profile of the oral film dosage form (pKP_{film}) is about 80% to about 125% of the pharmacokinetic profile of the corresponding intravenously delivered unit dosage form (pKP_{intravenously delivered}) at a confidence level of at least about 80%; wherein the pharmacokinetic profile of the film dosage form (pKP_{film}) is about 90% to about 115% of the pharmacokinetic profile of the corresponding intravenously delivered unit dosage form (pKP_{intravenously delivered}) at a confidence level of at least about 80%; wherein the pharmacokinetic profile of the film dosage form (pKP_{film}) is about 95% to about 110% of the pharmacokinetic profile of the corresponding intravenously delivered unit dosage form (pKP_{intravenously delivered}) at a confidence level of at least about 80%.

The present disclosure includes a pharmaceutically acceptable film for oral mucosal delivery of a biological macromolecule active in individual unit dosage forms including: a. the biological macromolecule active; b. a water-soluble or water swellable polymer matrix including: i. a polyethylene oxide composition, ii. water or a water-containing containing solvent and iii. silicon dioxide; wherein, when the individual unit dosage form is administered to a patient, one or more of the AUC and Cmax of the individual unit dosage form is about 80% to about 125% at a confidence level of at least about 80% of the AUC and/or Cmax of the corresponding intravenously delivered unit dosage form, and wherein each individual unit dosage form contains about 10% to about 10,000% more of the biological macromolecule active than the corresponding intravenously delivered unit dosage form.

The present disclosure includes a self-supporting film dosage form for oral mucosal delivery of a pharmaceutical active in individual dosage units including a water-soluble or water swellable film-forming polymer matrix containing a pharmaceutical active, wherein when the film dosage form is administered to the oral mucosa of a mammal, a pharmacokinetic profile of the pharmaceutical active is substantially equivalent to the pharmacokinetic profile of a corresponding enterally or parenterally delivered non-film dosage form containing the pharmaceutical active, and wherein the film dosage form for mucosal delivery contains about 0.5% to about 75% less active per individual dosage unit than the corresponding enterally or parenterally delivered non-film dosage form.

The present disclosure includes a pharmaceutically acceptable film for oral mucosal delivery of a pharmaceutical active including: a. a pharmaceutical active; and b. a water-soluble or water swellable film-forming polymer matrix including: i. a polyethylene oxide composition, ii. water or a water-containing solvent, iii. and silicon dioxide; wherein the oral film is an individual unit dosage form, wherein, when the individual unit dosage form is administered to the oral mucosa of a patient, one or more of the AUC and Cmax of the pharmaceutical active is about 80% to about 125% at a confidence level of at least about 80% of the AUC and/or Cmax of the corresponding enterally or parenterally delivered non-film dosage form, and wherein each individual unit dosage form contains about 0.5% to about 50% less active than the corresponding enterally or parenterally delivered non-film dosage form.

The disclosure includes a self-supporting film dosage form for oral mucosal delivery of a pharmaceutical active in individual dosage units including a water-soluble or water swellable film-forming polymer matrix containing a pharmaceutical active, wherein when the film dosage form is administered to the oral mucosa of a mammal, a pharmacokinetic profile of the pharmaceutical active is substantially equivalent to the pharmacokinetic profile of a dosage form containing the pharmaceutical active which is delivered to the rectal mucosa, and wherein the film dosage form for oral mucosal delivery contains about 0.5% to about 75% less active per individual dosage unit than a dosage form which is delivered to the rectal mucosa.

The dosage forms for mucosal delivery disclosed herein may be administered to a subject in a fed state or to a subject in a fasted state. The dosage forms for mucosal delivery disclosed herein which may be administered to a subject in a fed state or in a fasted state include the self-supporting film dosage forms for mucosal delivery and, more particularly, the self-supporting films for oral mucosal delivery disclosed herein. The subject may be a mammal. More particularly, the subject may be a patient such as a human patient.

A self-supporting film dosage form for mucosal delivery, preferably for delivery through the oral mucosa, of a pharmaceutical active is disclosed herein. The film is made in sheets and then cut into individual dosage units. The film contains a water-soluble or water swellable film-forming polymer matrix incorporating a pharmaceutical active. The pharmaceutical active may be a biological macromolecule or a small molecule. When the film dosage form is administered through the oral mucosa of a mammal, a pharmacokinetic profile of the pharmaceutical active is substantially equivalent to the pharmacokinetic profile of a corresponding enterally delivered dosage form containing the same pharmaceutical active, and the film dosage form for mucosal delivery contains about 0.5% to about 75% less active per individual dosage unit than the corresponding enterally delivered dosage form. When the film dosage form contains a biological macromolecule active and is administered through the oral mucosa of a mammal, a pharmacokinetic profile of the pharmaceutical active is substantially equivalent to the pharmacokinetic profile of a corresponding intravenously delivered dosage form containing the pharmaceutical active, and the film dosage form for mucosal delivery contains about 10% to about 10,000% more active per individual dosage unit than the corresponding intravenously delivered dosage form. When the film dosage form is administered through the oral mucosa of a mammal, a pharmacokinetic profile of the pharmaceutical active is substantially equivalent to the pharmacokinetic profile of a corresponding enterally or parenterally delivered non-film dosage form containing the same pharmaceutical active, and the film dosage form for mucosal delivery contains about 0.5% to about 75% less active per individual dosage unit than the corresponding enterally or parenterally delivered non-film dosage form.

Bioequivalence is generally determined and measured based on the same amount of a pharmaceutical active present in two different pharmaceutical dosage forms. As disclosed herein, it was surprisingly found that a substantially equivalent pharmacokinetic profile could be obtained for an active in a self-supporting film dosage form formulated for mucosal delivery as with an active in another individual unit dosage form formulated for delivery by another route, while the amount of active in the self-supporting film dosage form is different from the amount of active in the other unit dosage form. For small molecule actives, it was found that less of the active is needed in a self-supporting film dosage form formulated for mucosal delivery to obtain a substantially equivalent pharmacokinetic profile to that of a dosage form formulated for delivery by another route. For biological macromolecule actives, it was found that more of the active is needed in a self-supporting film dosage form formulated for mucosal delivery to obtain a substantially equivalent pharmacokinetic profile to that of an intravenously delivered dosage form.

It was found that a dosage form for mucosal delivery of the disclosure, such as a self-supporting film of the disclosure, can be either sub potent or super potent compared to other dosage forms such as enterally delivered dosage forms. In certain instances, a dosage form for mucosal delivery can be both sub potent and super potent in different pharmacokinetic parameters.

As used herein, a dosage form for mucosal delivery of the disclosure, such as a self-supporting film dosage form of the disclosure, is “sub potent” when it contains less active to achieve pharmacokinetic bioequivalence to another dosage form that contains a targeted amount of active.

As used herein, a dosage form for mucosal delivery of the disclosure, such as a self-supporting film dosage form of the disclosure, is “super potent” when it contains more active to achieve pharmacokinetic bioequivalence to another dosage form with less active.

### Fed/Fasted

A dosage form for mucosal delivery of the disclosure, such as a self-supporting film of the disclosure, can be administered to a subject in a fasted state or in a fed state. As used herein, the terms “fed state” and “fasted state” are in accordance with the definitions attributed to those terms by the Food and Drug Administration (FDA). As such, a dosage form for mucosal delivery of the disclosure, such as a self-supporting film dosage form of the disclosure, is administered to a subject, such as a human patient, in a fed state when it is delivered to the subject, such as a human patient, within 30 minutes of the subject, such as a human patient, consuming a high fat meal. A dosage form for mucosal delivery of the disclosure, such as a self-supporting film dosage form of the disclosure, is administered to a subject in a fasted stated, when it is administered to a subject, such as a human patient, in the absence of food or drink at least 8 hours prior to administration. Accordingly, a subject in a fasted state, such as a human patient in a fasted state, will not have consumed food or drink at least 8 hours prior to administration of the dosage form for mucosal delivery of the disclosure, such as a self-supporting film of the disclosure.

The term “film” can include films and sheets, in any shape, including rectangular, square, or other desired shape. A film can be any desired thickness and size. In an embodiment, a film can have a thickness and size such that it can be administered to a patient, for example, by placement into the oral cavity. A film can be relatively thin from about 0.0025 mm to about 0.250 mm, or a film can be somewhat thicker from about 0.250 mm to about 1.0 mm. Some films may be even thicker, e.g., greater than about 1.0 mm, or thinner, e.g., less than about 0.0025 mm. A film can be a single layer or a film can be multi-layered, including laminated or multiple cast films. When multilayered, the pharmaceutical active may be present in one layer, in more than one layer but not all layers, or in all layers. The active may be present in the mucosal-contacting layer. A pharmaceutical active may be combined, with a permeation active enhancer in a single layer, each contained in separate layers, or can each be otherwise contained in discrete regions of the same dosage form. In certain embodiments, the pharmaceutical active contained in the polymer matrix can be dispersed in the matrix. In certain embodiments, the permeation enhancer being contained in the polymer matrix can be dispersed in the matrix.

The individual dosage unit of the self-supporting film disclosed herein can have a suitable thickness, and small size, which is between about 0.0625 inch to about 3 inch by about 0.0625 inch to about 3 inch. In at least one aspect, the film size may be greater than about 0.0625 inch, greater than about 0.5 inch, greater than about 1 inch, greater than about 2 inches, about 3 inches, and greater than 3 about inches, less than about 3 inches, less than about 2 inches, less than about 1 inch, less than about 0.5 inch, less than about 0.0625 inch, and/or in another aspect, greater than about 0.0625 inch, greater than about 0.5 inch, greater than about 1 inch, greater than about 2 inches, or greater than about 3 inches, about 3 inches, less than about 3 inches, less than about 2 inches, less than about 1 inch, less than about 0.5 inch, less than about 0.0625 inch. The aspect ratio, including thickness, length, and width can be optimized by a person of ordinary skill in the art based on the chemical and physical properties of the polymer matrix, the pharmaceutical active, dosage, enhancer, and other additives involved as well as the dimensions of the desired dispensing unit. The self-supporting film should have good adhesion when placed in the buccal cavity or in the sublingual region of the patient.

The self-supporting film disclosed herein may dissolve (which includes dispersing and dissolving) in about 30 seconds to about 24 hours, about 30 seconds to about 30 minutes, about 1 minute to about 24 hours, about 1 minute to about 30 minutes, about 1 minute to about 20 minutes, about 3 minutes to about 40 minutes, or about 5 minutes to about 30 minutes. The self-supporting film disclosed herein may dissolve in more than about 1 minute, more than 5 about minutes, more than about 7 minutes, more than about 10 minutes, more than about 12 minutes, more than about 15 minutes, more than about 20 minutes, more than about 30 minutes, about 30 minutes, or less than about 30 minutes, less than about 20 minutes, less than about 15 minutes, less than about 12 minutes, less than about 10 minutes, less than about 7 minutes, less than about 5 minutes, or less than about 1 minute. Sublingual dissolution rates may be shorter than buccal dissolution rates.

More specifically, oral dissolving films can fall into three main classes: fast dissolving, moderate dissolving and slow dissolving. Oral dissolving films can also include a combination of any of the above categories. Fast dissolving films may dissolve in about 1 second to about 30 seconds in the mouth, including more than about 1 second, more than about 5 seconds, more than about 10 seconds, more than about 20 seconds, and less than about 30 seconds. Moderate dissolving films may dissolve in about 1 to about 30 minutes in the mouth, including more than about 1 minute, more than about 5 minutes, more than about 10 minutes, more than about 20 minutes, and less than about 30 minutes. Slow dissolving films may dissolve in more than about 30 minutes in the mouth, including about 30 minutes to about 24 hours, about 30 minutes to about 12 hours, about 30 minutes to about 10 hours, and about 1 hour to about 10 hours. Fast dissolving films may include (or consist of) low molecular weight hydrophilic polymers (e.g., polymers having a molecular weight between about 1,000 to about 9,000 daltons, or polymers having a molecular weight up to about 200,000 daltons). In contrast, slow dissolving films may include high molecular weight polymers (e.g., having a molecular weight in millions).

There may be certain advantages to moderate dissolving films in that they may dissolve rather quickly, while having a good level of mucoadhesion. Moderate dissolving films can also be flexible, quickly wettable, and are typically non-irritating to the patient. Such moderate dissolving films can provide a quick enough dissolution rate, for example, between about 1 minute and about 20 minutes, while providing an acceptable mucoadhesion level such that the film is not easily removable once it is placed in the oral cavity of the patient. This can ensure complete delivery of a pharmaceutical active to a patient.

Self-supporting means that the film maintains its integrity and structure in the absence of any separate support. The films of the present disclosure are formulated for absorption in the oral mucosa, that is, e.g., buccal or sublingual. Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms.

A film and/or its components may be water-soluble, water swellable or water-insoluble. The term “water-soluble” may refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water.

A film can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through controlled drying processes, which include application of heat and/or radiation energy to the wet film matrix to form a visco-elastic structure, thereby controlling the uniformity of content of the film. The controlled drying processes can include air alone, heat alone, or heat and air together contacting the top of the film, bottom of the film, or the substrate supporting the cast, or deposited or extruded film, or contacting more than one surface at the same time or at different times during the drying process. Some of such processes are described in more detail in U.S. Pat. Nos. 8,765,167 and 8,652,378, which are incorporated by reference herein. Alternatively, the films may be extruded as described in U.S. Patent Publication No. 2005/0037055 A1, which is incorporated by reference herein.

A polymer matrix included in the films may be water-soluble, water swellable, or a combination thereof.

The polymer matrix includes a polymer. The polymer may be a polyethylene oxide. The polymer may include cellulose, cellulose derivatives or gums. The polymer may include polyethylene oxide, cellulose, cellulose derivatives, or a combination thereof. The polymer may be a cellulosic polymer. In certain embodiments, the cellulosic polymer can be hydroxypropylmethyl cellulose, hydroxyethyl cellulose, hydroxyethylmethyl cellulose, hydroxypropyl cellulose, methylcellulose, carboxymethyl cellulose and/or sodium carboxymethylcellulose. In certain embodiments, the polymer can include hydroxypropyl methylcellulose. In certain embodiments, the polymer can include polyethylene oxide and hydroxypropyl methylcellulose. In certain embodiments, the polymer can include polyethylene oxide and/or polyvinyl pyrrolidone. In certain embodiments, the polymer matrix can include polyethylene oxide and/or a polysaccharide. In certain embodiments, the polymer matrix can include polyethylene oxide, hydroxypropyl methylcellulose and/or a polysaccharide. In certain embodiments, the polymer matrix can include polyethylene oxide, a cellulosic polymer, polysaccharide and/or polyvinylpyrrolidone. In certain embodiments, the polymer matrix can include at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide, propylene oxide co-polymers, collagen, albumin, poly-amino acids, polyphosphazenes, polysaccharides, chitin, chitosan, and derivatives thereof. Other examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, polysaccharides, and combinations thereof.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, films formed from such water-soluble polymers may be sufficiently water-soluble to be dissolvable upon contact with bodily fluids.

Other polymers useful for incorporation into the films include biodegradable polymers, copolymers, block polymers or combinations thereof. It is understood that the term “biodegradable” is intended to include materials that chemically degrade, as opposed to materials that physically break apart (i.e., bioerodable materials). The polymers incorporated in the films can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, polyhydroxy-alkanoates or mixtures thereof. The polymer matrix can include one, two, three, four or more components.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of pharmaceutical active contained in the composition. Some pharmaceutical actives may only require a few minutes for delivery via the mucosal membrane, whereas other pharmaceutical actives may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable. The inclusion of one or more polymers that are water-swellable, water-insoluble and/or biodegradable may provide films with slower dissolution or disintegration rates than films formed from water-soluble polymers alone. As such, the film may adhere to the mucosal membrane for longer periods of time, such as up to several hours, which may be desirable for delivery of certain pharmaceutical actives.

The polymer matrix may include a dendritic polymer which can include highly branched macromolecules with various structural architectures. The dendritic polymers can include dendrimers, dendronised polymers (dendrigrafted polymers), linear dendritic hybrids, multi-arm star polymers, or hyperbranched polymers.

The polymer matrix may include a hyperbranched polymer, which are highly branched polymers with imperfections in their structure. However they can be synthesized in a single step reaction which can be an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications. See, e.g., Dendrimers as Drug Carriers: Applications in Different Routes of Drug Administration. J Pharm Sci, VOL. 97, 2008, 123-143, which is incorporated by reference herein.

The dendritic polymers can have internal cavities which can encapsulate drugs. The steric hindrance caused by the highly dense polymer chains might prevent the crystallization of the drugs. Thus, branched polymers can provide additional advantages in formulating crystallizable drugs in a polymer matrix.

Examples of suitable dendritic polymers include but are not limited to poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers or hyperbranched polymers.

Other examples of hyperbranched polymers include poly(amines)s, polycarbonates, poly(ether ketone)s, polyurethanes, polycarbosilanes, polysiloxanes, poly(ester amines, poly(sulfone amine)s, poly(urea urethane)s and polyether polyols such as polyglycerols.

For instance, in some embodiments, the self-supporting film may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In an embodiment, the water-soluble polymer may be a hydrophilic cellulosic polymer, such as hydroxypropyl cellulose and/or hydroxypropylmethyl cellulose. In another embodiment, one or more water swellable, water-insoluble and/or biodegradable polymers also may be included in polyethylene oxide-based film. Any of the water-swellable, water-insoluble or biodegradable polymers provided above may be employed. The second polymer may be employed in amounts of about 0% to about 80% by weight of the polymer matrix, more specifically about 30% to about 70% by weight, and even more specifically about 40% to about 60% by weight, including greater than about 5%, greater than about 10%, greater than about 15%, greater than about 20%, greater than about 30%, greater than about 40%, greater than about 50%, greater than about 60%, and greater than about 70%, about 70%, less than about 70%, less than about 60%, less than about 50%, less than about 40%, less than about 30%, less than about 20%, less than about 10% or less than about 5% by weight.

Additives may be included in the dosage forms for mucosal delivery of the disclosure. Additives may be included in the polymer matrix, e.g., dispersed therein, or in the film composition that makes up the self-supporting film. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutical active(s) and/or the permeation enhancer, when present. As used herein, the term “stabilizer” means an excipient capable of preventing aggregation or other physical degradation, as well as chemical degradation, of the active pharmaceutical ingredient, another excipient, or the combination thereof.

Stabilizers may also be classified as antioxidants, sequestrants, pH modifiers, emulsifiers and/or surfactants, and UV stabilizers.

Antioxidants (i.e., pharmaceutically compatible compound(s) or composition(s) that decelerates, inhibits, interrupts and/or stops oxidation processes) suitable for use herein include, but are not limited to, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) suitable for use herein include, but are not limited to: calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by α linkages), cyclogalactins (5 or more β-D-galactopyranose units linked at the 1,4 positions by β linkages), cycloaltrins (5 or more α-D-altropyranose units linked at the 1,4 positions by a linkages), and combinations thereof.

pH modifiers suitable for use herein include, but are not limited to: acids (e.g., tartaric acid, citric acid, lactic acid, fumaric acid, phosphoric acid, ascorbic acid, acetic acid, succininc acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants suitable for use herein include, but are not limited to: poloxamers or pluronics, polyethylene glycols, polyethylene glycol monostearate, polysorbates, sodium lauryl sulfate, polyethoxylated and hydrogenated castor oil, alkyl polyoside, a grafted water-soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C₁₀-C₂₄)-alkyl-dimethylammonium chloride or bromide), (C₁₀-C₂₄)-alkyl-dimethylethylammonium chloride or bromide, (C₁₀-C₂₄)-alkyl-trimethylammonium chloride or bromide cetyltrimethylammonium chloride or bromide), (C₁₀-C₂₄)-alkyl-dimethylbenzylammonium chloride or bromide (e.g., (C₁₂-C₁₈)-alkyl-dimethylbenzylammonium chloride), N—(C₁₀-C₁₈)-alkyl-pyridinium chloride or bromide (e.g., N—(C₁₂-C₁₆)-alkyl-pyridinium chloride or bromide), N—(C₁₀-C₁₈)-alkyl-isoquinolinium chloride, bromide or monoalkyl sulfate, N—(C₁₂-C₁₈)-alkyl-polyoylaminoformylmethylpyridinium chloride, N—(C₁₂-C₁₈)-alkyl-N-methylmorpholinium chloride, bromide or monoalkyl sulfate, N—(C₁₂-C₁₈)-alkyl-N-ethylmorpholinium chloride, bromide or monoalkyl sulfate, (C₁₆-C₁₈)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate the foregoing, “acyl” standing for, e.g., stearyl or oleyl), and combinations thereof.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers suitable for use herein include, but are not limited to: ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-carnosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, o-phenanthroline, and citrate. “Detergents” include, but are not limited to: SDS and sodium lauroyl sarcosyl. “Chaotropes” include, but are not limited to guandinium hydrochloride, isothiocyanate, urea, and formamide. As discussed herein, stabilizers can be present in about 0.0001% to about 50% by weight, including greater than about 0.0001%, greater than about 0.001%, greater than about 0.01%, greater than about 0.1%, greater than about 1%, greater than about 5%, greater than about 10%, greater than about 20%, greater than about 30%, greater than about 40%, greater than about 50%, less than about 50%, less than about 40%, less than about 30%, less than about 20%, less than about 10%, less than about 1% less than about 0.1%, less than about 0.01%, less than about 0.001%, or less than about 0.0001% by weight.

Other additives suitable for use herein include, but are not limited to: gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

The amount of additives in the film can range up to about 80%, about 0.005% to about 50%, about 1% to about 20%, or about 3% to about 20% based on the weight of the film composition (which is the totaled weight of all components therein) including greater than about 1%, greater than about 5%, greater than about 10%, greater than about 20%, greater than about 30%, greater than about 40%, greater than about 50%, greater than about 60%, less than about 80%, less than about 70%, less than about 60%, less than about 50%, less than about 40%, less than about 30%, less than about 20%, less than about 10%, less than about 5%, about 3%, or less than about 1%.

Other additives can include anti-tacking, flow agents and opacifiers, such as the oxides of magnesium aluminum, silicon, titanium, etc. These additives may be present in a concentration range of about 0.005% to about 5%, or about 0.02% to about 2% based on the weight of the film composition, including greater than about 0.02%, greater than about 0.2%, greater than about 0.5%, greater than about 1%, greater than about 1.5%, greater than about 2%, greater than about 4%, about 5%, greater than about 5%, less than about 4%, less than about 2%, less than about 1%, less than about 0.5%, less than about 0.2%, or less than about 0.02%.

In certain embodiments, the film may include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols sorbitol, sodium diethylsulfosuccinate, triethyl citrate, tributyl citrate, phytoextracts, fatty acid esters, fatty acids, oils and the like. Plasticizers may be added in concentrations ranging from about 0.1% to about 40%, or about 0.5% to about 20% based on the weight of the film composition, including greater than about 0.5%, greater than about 1%, greater than about 1.5%, greater than about 2%, greater than about 4%, greater than about 5%, greater than about 10%, greater than about 15%, about 20%, greater than about 20%, less than about 20%, less than about 15%, less than about 10%, less than about 5%, less than about 4%, less than about 2%, less than about 1%, and less than about 0.5%. There may further be added compounds to improve the texture properties of the film material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids. The total amounts used of the fats, mono-, di-glycerides and/or lecithins are up to about 5% or within the range of about 0.5% to about 2% based on the weight of the film composition.

The film composition can also include compounds to improve the textural properties of the self-supporting film.

Other ingredients can include binders which contribute to the ease of formation and general quality of the film. Non-limiting examples of binders suitable for use herein include, but are not limited to: starches, natural gums, pregelatinized starches, gelatin, polyvinylpyrrolidone, methylcellulose, sodium carboxymethylcellulose, ethylcellulose, polyacrylamides, polyvinyloxoazolidone, and polyvinylalcohols.

Further potential additives suitable for use herein include, but are not limited to: solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutical actives may fit within the hydrophobic cavity, thereby producing an inclusion complex, which is soluble in water. Accordingly, the formation of the inclusion complex permits very insoluble and/or unstable pharmaceutical actives to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of the film composition.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform films. Agents or components which perform this function can be referred to as densification or densifying agents. As described above, entrapped air or undesired components may lead to non-uniform films.

A variety of other components and fillers may also be added to the films disclosed herein. These may include, without limitation, surfactants; other anti-foaming agents; such as simethicone, which promote a smoother film surface by releasing oxygen from the film; thermo-setting gels such as pectin, carageenan, and gelatin, which help in maintaining the dispersion of components; and inclusion compounds, such as cyclodextrins and caged molecules, which improve the solubility and/or stability of certain active components.

Further additives may be inorganic fillers, such as the oxides of magnesium aluminum, silicon, titanium, etc. desirably in a concentration range of about 0.02% to about 3% by weight and desirably about 0.02% to about 1% based on the weight of the film composition.

It may be useful to add silicon dioxide, calcium silicate, or titanium dioxide in a concentration of about 0.02% to about 1% by weight of the total composition. These compounds act as texturizing agents.

These additives are to be used in amounts sufficient to achieve their intended purpose. Generally, the combination of certain of these additives will alter the overall release profile of the active ingredient and can be used to modify, i.e., impede or accelerate the release.

Lecithin is one surface active agent for use in the present invention. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

As additional modifiers which enhance the procedure and product of the present invention are identified, Applicants intend to include all such additional modifiers within the scope of the invention claimed herein.

Any other optional components described in commonly assigned U.S. Pat. Nos. 7,425,292 and 8,765,167, referred to above, also may be included in the films described herein.

A buffer may be included in the dosage forms for mucosal delivery of the disclosure. The film further desirably contains a buffer so as to control the pH. Any desired level of buffer tray be incorporated into the polymer matrix so as to provide the desired pH level encountered as the pharmaceutical active is released from the film. The buffer is preferably provided in an amount sufficient to control the release from the film and/or the absorption into the body of the active. The buffer may include sodium citrate, citric acid, bitartrate salt, or any combination thereof.

The self-supporting films described herein may be formed via any desired process. Suitable processes are set forth in U.S. Pat. Nos. 8,652,378, 7,425,292 and 7,357,891, which are incorporated by reference herein. In one embodiment, the film is formed by first preparing a wet composition, the wet composition including a polymer matrix and a therapeutically effective amount of a pharmaceutical active. The wet composition is cast into a film and then sufficiently dried to form a self-supporting film. The wet composition may be cast into individual dosages, or it may be cast into a sheet, which is then cut into individual unit dosages.

As used herein, the term “dosage form for mucosal delivery” refers to any dosage form that delivers an active through a mucous membrane, such as a mucous membrane in the mouth, nose, vagina and rectum. Accordingly, the “mucosal delivery” as used herein includes oral mucosal delivery, nasal mucosal delivery, aural mucosal delivery, ocular mucosal delivery, vaginal mucosal delivery and rectal mucosal delivery. A variety of dosage forms can be used for mucosal delivery including, without limitation, films, sprays, aerosols, nebulizers, gels, tablets, patches, powders and liquids. Many of the same forms may be used for enteral delivery as well.

The self-supporting film disclosed herein can adhere to a mucosal membrane. The self-supporting film may be used in the localized treatment of body tissues, diseases, or wounds which may have moist surfaces and which are susceptible to bodily fluids, such as the mouth, the vagina, organs, or other types of mucosal membranes. The self-supporting film carries a pharmaceutical active, and upon application and adherence to the mucosal membrane, offers a layer of protection and delivers the pharmaceutical active to the treatment site, the surrounding tissues, and other bodily fluids. The self-supporting film provides an appropriate residence time for effective drug delivery at the treatment site, given the control of erosion in aqueous solution or bodily fluids such as saliva, and the slow, natural erosion of the film concomitant or subsequent to the delivery. “Mucosal” or “mucosal membrane” as used interchangeably herein refer to the mucus-coated biological membranes of the body. In certain embodiments, the mucosal membrane is in the oral cavity, including buccal, sublingual, gingival, and palatal. Absorption through a “mucosal membrane” or “mucosal delivery” or “transmucosal delivery” encompasses all forms of delivery through a mucosal membrane. “Oral transmucosal” delivery of an active includes delivery across any tissue of the mouth, pharynx, larynx, trachea, or upper gastrointestinal tract, particularly including the sublingual, gingival and palatal mucosal tissues.

As used herein, “sublingual” means literally “under the tongue” and refers to a method of administering substances via the mouth in such a way that the substances are rapidly absorbed via the blood vessels under the tongue rather than via the digestive tract. Among the various transmucosal sites, the mucosa of the sublingual cavity is found to be the most convenient and easily accessible site for the delivery of therapeutic agents for both local and systemic delivery as controlled release dosage forms because it of its abundant vascularization and the near absence of Langerhans cells. Direct access to the systemic circulation through the internal jugular vein bypasses the hepatic first pass metabolism leading to high bioavailability. Further, owing to the highly vascularized nature of the sublingual mucosal membrane and the reduced number of epithelial cell layers compared to other mucosal membranes, absorption of therapeutic substances occurs rapidly, thus allowing for direct access to the systemic circulation and thus enable quick onset of action while avoiding complications of oral administration.

The residence time of the self-supporting film depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water-soluble organic and inorganic salts; by using crosslinking agents such as glyoxal with polymers such as hydroxyethyl cellulose for partial crosslinking; or by post-treatment irradiation or curing, which may alter the physical state of the film, including its crystallinity or phase transition, once obtained. These strategies might be employed alone or in combination in order to modify the erosion kinetics of the film.

Upon application, the self-supporting film adheres to the mucosal membrane and is held in place. Water absorption softens the film, thereby diminishing the foreign body sensation. As the film rests on the mucosal membrane, delivery of the drug occurs. Residence times may be adjusted over a wide range depending upon the desired timing of the delivery of the chosen pharmaceutical active and the desired lifespan of the carrier. Generally, however, the residence time is modulated between about a few seconds to about a few days. Preferably, the residence time for most pharmaceutical actives is adjusted from about 5 seconds to about 24 hours. More preferably, the residence time is adjusted from about 5 seconds to about 30 minutes. In addition to providing drug delivery, once the self-supporting film adheres to the mucosal membrane, it also provides protection to the treatment site, acting as an erodable bandage. Lipophilic agents can be designed to slow down erodability to decrease disintegration and dissolution.

It is also possible to adjust the kinetics of erodability of the self-supporting film by adding excipients which are sensitive to enzymes such as amylase, very soluble in water such as water-soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the film.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, ally (ethers, alkyl aryl ethers, sorbitan monostearate, sorbitan monooleate and/or sorbitan monopalmitate.

In addition to the active, any of the additives listed above may be added and dispersed or dissolved uniformly in the polymer matrix. The active and flavoring agents, when present, can be incorporated before or after film forming.

The thickness of the self-supporting film disclosed herein may vary, depending on the thickness of each of the layers and the number of layers. Both the thickness and number of layers (i.e., one layer or multi-layered, e.g., two, three, four or more) may be adjusted in order to vary the erosion kinetics. If the self-supporting film has two layers, the total film thickness may range from about 0.005 mm to about 2 mm, from about 0.01 mm to about 1 mm, or from about 0.1 mm to about 0.5 mm. The total film thickness may be greater than about 0.1 mm, greater than about 0.2 mm, about 0.5 mm, greater than about 0.5 mm, less than about 0.5 mm, less than about 0.2 mm, or less than about 0.1 mm. The thickness of each layer may vary from about 10% to about 90%, or from about 30% to about 60%, of the total thickness of the layered self-supporting film. Any one layer may be greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, or less than 10% of the total thickness of the layered self-supporting film. The preferred thickness of each layer may vary from about 0.01 mm to about 0.9 mm, or from about 0.03 mm to about 0.5 mm.

As one skilled in the art will appreciate, when systemic delivery, e.g., transmucosal or transdermal delivery is desired, the treatment site may include any mucosal area in which the film is capable of delivery and/or maintaining a desired level of pharmaceutical active in the blood, lymph, or other bodily fluid. Typically, such treatment sites include the oral, aural, ocular, anal, nasal, and vaginal mucosal tissue. In an embodiment, the treatment sites are the oral mucosa.

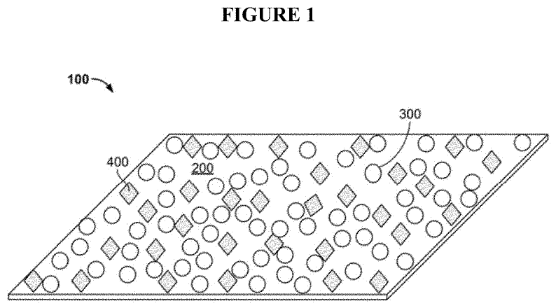

The carrier may be any suitable carrier known in the art. Carriers for use herein include, without limitation, matrices, such as polymeric matrices, micelles, complexes and liquid carriers. Referring to FIG. 1, a self-supporting individual film dosage form 100 comprises a polymer matrix 200 with a pharmaceutical active 300 and permeation enhancer 400 contained in the polymer matrix.

The enterally delivered dosage form as used herein is any dosage form that is known in the art for delivery of an active orally via absorption though the gastrointestinal tract. For example, the enterally delivered dosage form may be selected from the group consisting of tablets, capsules, lozenges, troches, wafers, caplets, chewables, gels, liquids, orally dissolving tablets (ODTs), lyophilized dosage forms and any combination thereof. The term “corresponding enterally delivered dosage form” means that the enterally delivered dosage form contains the same active as the dosage form for mucosal delivery to which it is being compared. When compared to a self-supporting film dosage form, the term “corresponding enterally delivered dosage form” means that the enterally delivered dosage form contains the same active as the self-supporting film dosage form to which it is being compared.

The parenterally delivered dosage form as used herein is any dosage form that is known in the art for delivery of an active by any route other than orally for absorption though the gastrointestinal tract. For example, the parenterally delivered dosage form may be formulated for the following routes of administration: intramuscular, subcutaneous, intravenous, intranasal, intravaginal, transdermal, transmucosal. The parenterally delivered dosage form may be selected from the group consisting of tablets, patches, films, capsules, lozenges, troches, wafers, caplets, chewables, gels, liquids, orally dissolving tablets (ODTs), lyophilized dosage forms and any combination thereof. The term “corresponding parenterally delivered dosage form” means that the parenterally delivered dosage form contains the same active as the self-supporting film dosage form to which it is being compared.

The term “corresponding intravenously delivered dosage form” refers to any known intravenously delivered dosage form containing the same active as the dosage form for mucosal delivery to which it is being compared. When the corresponding intravenously delivered dosage form is being compared to a self-supporting film dosage form, the corresponding intravenously delivered dosage form refers to any known intravenously delivered dosage form containing the same active as the self-supporting film dosage form to which it is being compared.

### Pharmaceutical Active

The dosage forms for mucosal delivery disclosed herein, such as the self-supporting films disclosed herein, may be used as a vehicle for effective delivery of a wide range of pharmaceutical actives. Embodiments provide improved dosage forms to deliver actives that are appropriate for all age groups and that physician, parents, patients and family members can administer easily. These dosage forms are economical to prepare and have an extended shelf life. They are easy to handle and non-tacky before administration so as to avoid disintegration prior to use and are conveniently packaged for shelf life, ease of storage and distribution. When the dosage form is a film for mucosal delivery, the dosage form may be administered to the subject by placing the film on a mucous surface, at which time the film becomes a mucoadhesive coating, characterized by the property that it can no longer exist in an independent form and is subsequently dispersed in solution. Embodiments provide a delivery system for pharmaceutical actives and other active agents that will dissolve and completely release their contents on a moist mucosal surface—for example in the oral cavity. The release of the pharmaceutical active occurs without mastication or the need for intake of water.

The pharmaceutical active comprises from about 0.001% to about 99%, from about 0.003% to about 75%, or from about 0.005% to about 50% by weight of the dosage form for mucosal delivery including, more than about 0.005%, more than about 0.05%, more than about 0.5%, more than about 1%, more than about 5%, more than about 10%, more than about 15%, more than about 20%, more than about 30%, about 50%, more than about 50%, less than about 50%, less than about 30%, less than about 20%, less than about 15%, less than about 10%, less than about 5%, less than about 1%, less than about 0.5%, less than about 0.05%, or less than about 0.005%. The amounts of other components may vary depending on the active and other components but typically these components comprise no more than about 50%, no more than about 30%, or no more than about 15% by total weight of the dosage form for mucosal delivery.

When the dosage form for mucosal delivery is a film, the amount of pharmaceutical active in the film depends on the active, the desired treatment strength and the number of layers of the film, as readily understood by one of ordinary skill in the art. The pharmaceutical active comprises from about 0.001% to about 99%, from about 0.003% to about 75%, or from about 0.005% to about 50% by weight of the film composition including, more than about 0.005%, more than about 0.05%, more than about 0.5%, more than about 1%, more than about 5%, more than about 10%, more than about 15%, more than about 20%, more than about 30%, about 50%, more than about 50%, less than about 50%, less than about 30%, less than about 20%, less than about 15%, less than about 10%, less than about 5%, less than about 1%, less than about 0.5%, less than about 0.05%, or less than about 0.005%. The amounts of other components may vary depending on the active and other components but typically these components comprise no more than about 50%, no more than about 30%, or no more than about 15% by total weight of the film composition.

At least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the pharmaceutical active in the individual dosage form for oral delivery is absorbed via the oral mucosa.

At least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99% of the pharmaceutical active in the individual dosage form of the self-supporting film is absorbed via the oral mucosa.

Each individual unit dosage form for mucosal delivery, such as each individual unit film dosage form for mucosal delivery, contains about 0.5% to about 75% less pharmaceutical active per individual dosage unit than the corresponding enterally delivered dosage form to achieve a substantially equivalent pharmacokinetic profile. The pharmaceutical active may be one or more small molecules, one or more biological macromolecules or a combination thereof. Each individual unit dosage form for mucosal delivery, such as each individual unit film dosage form for mucosal delivery, may contain about 0.5% to about 75% less, about 0.5% to about 50% less, about 0.5% to about 25% less, about 0.5% to about 20% less, about 1% to about 15% less, about 1% to about 10% less, about 5% to about 75% less, about 5% to about 50% less, about 5% to about 25% less, about 5% to about 15% less, about 10% to about 50% less, about 10% to about 25% less, about 10% to about 20% less, or about 15% to about 30% less active per individual dosage unit than the corresponding enterally delivered dosage form to achieve a substantially equivalent pharmacokinetic profile. In other embodiments thereof, each individual unit dose for mucosal delivery, such as each individual unit film dosage form for mucosal delivery, contains more than about 0.5%, more than about 1%, more than about 5%, more than about 10%, more than about 15%, more than about 20%, more than about 25%, more than about 40%, more than about 50%, or about 1%, about 2%, about 5%, about 10%, about 15%, about 20%, about 25%, about 30%, about 40%, about 50%, or about 60% less active per individual dosage unit than the corresponding enterally delivered dosage form to achieve a substantially equivalent pharmacokinetic profile.

When the active is a small molecule, each individual unit dosage form for mucosal delivery, such as each individual unit film dosage form for mucosal delivery, may contain about 0.5% to about 75% less, about 0.5% to about 50% less, about 0.5% to about 25% less, about 0.5% to about 20% less, about 1% to about 15% less, about 1% to about 10% less, about 5% to about 75% less, about 5% to about 50% less, about 5% to about 25% less, about 5% to about 15% less, about 10% to about 50% less, about 10% to about 25% less, about 10% to about 20% less, or about 15% to about 30% less active per individual dosage unit than the corresponding enterally delivered dosage form to achieve a substantially equivalent pharmacokinetic profile. In other embodiments thereof, each individual unit dosage form for mucosal delivery, such as each individual unit film dosage form for mucosal delivery, contains more than about 0.5%, more than about 1%, more than about 5%, more than about 10%, more than about 15%, more than about 20%, more than about 25%, more than about 40%, more than about 50%, or about 1%, about 2%, about 5%, about 10%, about 15%, about 20%, about 25%, about 30%, about 40%, about 50%, or about 60% less active per individual dosage unit than the corresponding enterally delivered dosage form to achieve a substantially equivalent pharmacokinetic profile.

When the active is a biological macromolecule, each individual unit dosage form for mucosal delivery, such as each individual film unit dosage form for mucosal delivery, may contain about 5% to about 10,000% more of the active than the corresponding intravenously delivered unit dosage form to achieve a substantially equivalent pharmacokinetic profile. In certain embodiments thereof, each individual unit dosage form for mucosal delivery, such as each individual unit film dosage form for mucosal delivery, contains about 10% to about 10,000% more, about 10% to about 1,000% more, about 10% to about 500% more, about 10% to about 200% more, about 10% to about 100% more, about 50% to about 1,000% more, about 50% to about 500% more, about 50% to about 200% more, about 50% to about 100% more, about 100% to about 10,000% more, about 100% to about 1,000% more, about 100% to about 500% more, about 500% to about 10,000% more, about 500% to about 1,000% more, or about 1,000% to about 2,000% more biological macromolecule active per individual dosage unit than the corresponding intravenously delivered dosage form to achieve a substantially equivalent pharmacokinetic profile. In other embodiments thereof, each individual unit film dosage form for mucosal delivery contains more than about 10%, more than about 20%, more than about 50%, more than about 100%, more than about 200%, more than about 500%, or about 10%, about 50%, about 100%, about 200%, about 500%, about 1,000%, about 2,000% more biological macromolecule active per individual dosage unit than the corresponding intravenously delivered dosage form to achieve a substantially equivalent pharmacokinetic profile.

In another embodiment, each individual unit dosage form for mucosal delivery, such as each individual unit film dosage form for mucosal delivery, may contain about 0.5% to about 75% less, about 0.5% to about 50% less, about 0.5% to about 25% less, about 0.5% to about 20% less, about 1% to about 15% less, about 1% to about 10% less, about 5% to about 75% less, about 5% to about 50% less, about 5% to about 25% less, about 5% to about 15% less, about 10% to about 50% less, about 10% to about 25% less, about 10% to about 20% less, or about 15% to about 30% less active per individual dosage unit than the corresponding enterally or parenterally delivered non-film dosage form to achieve a substantially equivalent pharmacokinetic profile. In other embodiments thereof, each individual unit dosage form for mucosal delivery, such as each individual unit film dosage form for mucosal delivery, contains more than about 0.5%, more than about 1%, more than about 5%, more than about 10%, more than about 15%, more than about 20%, more than about 25%, more than about 40%, more than about 50%, or about 1%, about 2%, about 5%, about 10%, about 15%, about 20%, about 25%, about 30%, about 40%, about 50%, or about 60% less active per individual dosage unit than the corresponding enterally or parenterally delivered non-film dosage form to achieve a substantially equivalent pharmacokinetic profile.

Suitable actives for use in the self-supporting films herein include, but are not limited to, the following therapeutic classes: ace-inhibitor; adrenergic agent; adrenocortical steroid; adrenocortical suppressant; aldosterone antagonist; alkaloid; amino acid; anabolic; analeptic; analgesic; anesthetic; anorectic; anti-acne agent; anti-adrenergic; anti-allergic; anti-amebic; anti-anemic; anti-anginal; anti-anxiety; anti-arthritic; anti-arrythmia; anti-asthmatic; anti-atherosclerotic; anti-cholesterolemic; antibacterial; antibiotic; anticholinergic; anticoagulant; anticonvulsant; antidepressant; antidiabetic; antidiarrheal; antidiuretic; antidote; anti-emetic; anti-epileptic; antifibrinolytic; antifungal; antihemorrhagic; antihistamine; antihyperlipidemia; antihypertensive; antihypotensive; anti-infective (both systemic and non-systemic); anti-inflammatory; anti-lipid; anti-manic; antimicrobial; antimigraine; antimitotic; antimycotic, antinauseant; antineoplastic; antineutropenic; anti-obesity; antiparasitic; anti-parkinson; antiproliferative; antipsychotic; anti-pyretic; antirheumatic; antiseborrheic; antisecretory; antispasmodic; anti-stroke; antithrombotic; anti-thyroid; anti-tumor; anti-tussive; anti-ulcerative; anti-uricemic; antiviral; appetite suppressant; appetite stimulant; biological response modifier; blood glucose regulator; blood modifier; blood metabolism regulator; bone resorption inhibitor; bronchodilator; cardiovascular agent; central nervous system stimulant; cerebral dilator; contraceptive; coronary dilator; cholinergic; cough suppressant; decongestant; depressant; diagnostic aid; dietary supplement; diuretic; dopaminergic agent; enzymes; estrogen receptor agonist; endometriosis management agent; expectorant; erectile dysfunction therapy; erythropoietic; ibrinolytic; fertility agent; fluorescent agent; free oxygen radical scavenger; gastric acid suppressant; gastrointestinal motility effector; genetic modifier; glucocorticoid; hair growth stimulant; hemostatic; histamine H2 receptor antagonists; homeopathic remedy; hormone; hypercalcemia management agent; hypocalcemia management agent; hypocholesterolemic; hypoglycemic; hypolipidemic; hypotensive; ion exchange resin; imaging agent; immunizing agent; immunomodulator; immunoregulator; immunostimulant; immunosuppressant; keratolytic; laxative; LHRH agonist; mood regulator; motion sickness preparation; mucolytic; muscle relaxant; mydriatic; nasal decongestant; neuromuscular blocking agent; neuroprotective; NMDA antagonist; non-hormonal sterol derivative; osteoporosis therapy; oxytocic; parasympatholytic; parasympathomimetic; plasminogen activator; platelet activating factor antagonist; platelet aggregation inhibitor; prostaglandin; psychotherapeutic; psychotropic; radioactive agent; respiratory agent; scabicide; sclerosing agent; sedative; sedative-hypnotic; selective adenosine A1 antagonist; serotonin antagonist; serotonin inhibitor; serotonin receptor antagonist; smoking cessation therapy; steroid; stimulant; sympatholytic; terine relaxant; thyroid hormone; thyroid inhibitor; thyromimetic; tranquilizer; tremor therapy; amyotrophic lateral sclerosis agent; cerebral ischemia agent; Paget's disease agent; unstable angina agent; vasoconstrictor; vasodilator; weight management; wound healing agent; xanthine oxidase inhibitor; and combinations thereof.

Examples of pharmaceutical actives suitable for use herein include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (commercially available as Oxycontin®); ibuprofen (commercially available as Motrin®, Advil®, Motrin Children's®, Motrin TB®, Advil Children's®, Motrin Infants'®, Motrin Junior®, Ibu-2®, Proprinal®, Ibu-200®, Midol Cramp Formula®, Bufen®, Motrin Migraine Pain®, Addaprin® and Haltran®), aspirin (commercially available as Empirin®, Ecotrin®, Genuine Bayer®, and Halfprin®), acetaminophen (commercially available as Silapap Infant's®, Silapap Children's®, Tylenol®, Tylenol Children's®, Tylenol Extra Strength®, Tylenol Infants' Original®, Tylenol Infants'®, Tylenol Arthritis®, T-Painol®, Q-Pap®, Cetafen®, Dolono®, Tycolene®, APAP® and Aminofen®), and combinations thereof that may optionally include caffeine. Other pain relieving agents may be used in the present invention, including meperidine hydrochloride (commercially available as Demerol®), capsaicin (commercially available as Qutenza®), morphine sulfate and naltrexone hydrochloride (commercially available as Embeda®), hydromorphone hydrochloride (commercially available as Dilaudid®), propoxyphene napsylate and acetaminophen (commercially available as Darvocet-N®), Fentanyl (commercially available as Duragesic®, Onsolis®, and Fentora®), sodium hyaluronate (commercially available as Euflexxa®), adalimumab (commercially available as Humira®), sumatriptan succinate (commercially available as Imitrex®), fentanyl iontophoretic (commercially available as Ionsys®), orphenadrine citrate (commercially available as Norgesic®), magnesium salicylate tetrahydrate (commercially available as Novasal®), oxymorphone hydrochloride (commercially available as Opana ER®), methocarbamol (commercially available as Robaxin®), carisoprodol (commercially available as Soma®), tramadol hydrochloride (commercially available as Ultracet® and Ultram®), morphine sulfate (commercially available as MS Contin®), metaxalone (commercially available as Skelaxin®), oxycodone hydrochloride (commercially available as OxyContin®), acetaminophen/oxycodone hydrochloride (commercially available as Percocet®), oxycodone/aspirin (commercially available as Percodan®), hydrocodone bitartrate/acetaminophen (commercially available as Vicodin®), hydrocodone bitartrate/ibuprofen (commercially available as Vicoprofen®), nepafenac (commercially available as Nevanac®), and pregabalin (commercially available as Lyrïca®).

The dosage forms for mucosal delivery disclosed herein, such as the self-supporting films disclosed herein, may further include agents such as NSAIDs, including etodolac (commercially available as Lodine®), ketorolac tromethamine (commercially available as Acular® or Acuvail®), naproxen sodium (commercially available as Anaprox®, Naprosyn®), flurbiprofen (commercially available as Ansaid®), diclofenac sodium/misoprostol (commercially available as Arthrotec®), celecoxib (commercially available as Celebrex®), sulindac (commercially available as Clinoril®), oxaprozin (commercially available as Daypro®), piroxicam (commercially available as Feldene®), indomethacin (commercially available as Indocin®), meloxicam (commercially available as Mobic®), mefenamic acid (commercially available as Ponstel®), tolmetin sodium (commercially available as Tolectin®), choline magnesium trisalicylate (commercially available as Trilisate®), diclofenac sodium (commercially available as Voltaren®), diclofenac potassium (commercially available as Cambia® or Zipsor®), and misoprostol (commercially available as Cytotec®). Opiate agonists and antagonists, such as buprenorphine and naloxone are further examples of drugs for use in the present invention.

Other preferred drugs for other preferred active ingredients for use herein include anti-diarrheals such as loperamide (commercially available as Imodium AD®, Imotil®, Kaodene®, Imperim®, Diamode®, QC Anti-Diarrheal®, Health Care America Anti-Diarrheal®, Leader A-D®, and Imogen®), nitazoxanide (commercially available as Alinia®) and diphenoxylate hydrochloride/atropine sulfate (commercially available as Lomotil®), anti-histamines, anti-tussives, decongestants, vitamins, and breath fresheners. Common drugs used alone or in combination for colds, pain, fever, cough, congestion, runny nose and allergies, such as acetaminophen, ibuprofen, chlorpheniramine maleate, dextromethorphan, dextromethorphan HBr, phenylephrine HCl, pseudoephedrine HCl, diphenhydramine and combinations thereof, such as dextromethophan HBr and phenylephrine HCl (available as Triaminic®) may be included in the film compositions of the present invention.

Other active agents useful herein include, but are not limited to, alcohol dependence treatment, such as acamprosate calcium (commercially available as Campral®); Allergy treatment medications, such as promethazine hydrochloride (commercially available as Phenergan®), bepotastine besilate (commercially available as Bepreve®), hydrocodone polistirex/chlorpheniramine polistirex (commercially available as Tussionex®), cetirizine hydrochloride (commercially available as Zyrtec®), cetirizine hydrochloride/pseudoephedrine hydrochloride (commercially available as Zyrtec-D®), promethazine hydrochloride/codeine phosphate (commercially available as Phenergan® with Codeine), pemirolast (commercially available as Alamast®), fexofenadine hydrochloride (commercially available as Allegra®), meclizine hydrochloride (commercially available as Antivert®), azelastine hydrochloride (commercially available as Astelin®), nizatidine (commercially available as Axid®), desloratadine (commercially available as Clarinex®), cromolyn sodium (commercially available as Crolom®), epinastine hydrochloride (commercially available as Elestat®), azelastine hydrochloride (commercially available as Optivar®), prednisolone sodium phosphate (commercially available as Orapred ODT®), olopatadine hydrochloride (commercially available as Patanol®), ketotifen fumarate (commercially available as Zaditor®), and montelukast sodium (commercially available as Singulair®); and anti-histamines such as diphenhydramine HCl (available as Benadryl®), loratadine (available as Claritin®), astemizole (available as Hismanal®), nabumetone (available as Relafen®), diphenydramine HCL (available as TheraFlu®) and clemastine (available as Tavist®).

Dosage forms for mucosal delivery of the present disclosure, such as films of the present disclosure, may further include Alzheimer's treatment medications, such as tacrine hydrochloride (commercially available as Cognex®), galantamine (commercially available as Razadyne®), donepezil hydrochloride (commercially available as Aricept®), rivastigmine tartrate (commercially available as Exelon®), caprylidene (commercially available as Axona®), and memantine (commercially available as Namenda®); anemia medication, such as cyanocobalamin (commercially available as Nascobal®) and ferumoxytol (commercially available as Feraheme®); anesthetics, such as antipyrine with benzocaine (commercially available as Auralgan®, Aurodex® and Auroto®); angina medication, such as amlodipine besylate (commercially available as Norvasc®), nitroglycerin (commercially available as Nitro-Bid®, Nitro-Dur®, Nitrolingual®, Nitrostat®, Transderm-Nitro®), isosorbide mononitrate (commercially available as Imdur®), and isosorbide dinitrate (commercially available as Isordil®); anti-tussives such as guaifensin; anti-Alzheimer's agents, such as nicergoline; and Ca^{H}-antagonists such as nifedipine (commercially available as Procardia® and Adalat®).

Actives useful in the present disclosure may also include anti-asthmatics, such as albuterol sulfate (commercially available as Proventil®), ipratropium bromide (commercially available as Atrovent®), salmeterol xinafoate (commercially available as Serevent®), zafirlukast (commercially available as Accolate®), flunisolide (commercially available as AeroBid®), metaproterenol sulfate (commercially available as Alupent®), albuterol inhalation (commercially available as Ventolin®), terbutaline sulfate (commercially available as Brethine®), formoterol (commercially available as Foradil®), cromolyn sodium (commercially available as Intal®), levalbuterol hydrochloride (commercially available as Xopenex®), zileuton (commercially available as Zyflo®), fluticasone propionate/salmeterol (commercially available as Advair®), albuterol sulfate/triamcinolone acetonide (commercially available as Azmacort®), dimethylxanthine (commercially available as Theophylline®), and beclomethasone (commercially available as Beclovent®, Beconase®, Qvar®, Vancenase®, Vanceril®); angioedema medication, such as C1 esterase Inhibitor (human) (commercially available as Berinert®) and ecallantide (commercially available as Kalbitor®); and antibacterial medications, such as trimethoprim/sulfamethoxazole (commercially available as Bactrim®), mupirocin (commercially available as Bactroban®), metronidazole (commercially available as Flagyl®), sulfisoxazole acetyl (commercially available as Gantrisin®), bismuth subsalicylate and metronidazole/tetracycline hydrochloride (commercially available as Helidac Therapy®), nitrofurantoin (commercially available as Macrodantin®), norfloxacin (commercially available as Noroxin®), erythromycin ethylsuccinate/Sulfisoxazole acetyl (commercially available as Pediazole®), and levofloxacin (commercially available as Levaquin®).

The dosage forms of the present disclosure, such as the films of the present disclosure, may further include one or more antibiotics, including amoxicillin (commercially available as Amoxil®), ampicillin (commercially available as Omnipen®, Polycillin® and Principen®), amoxicillin/clavulanate potassium (commercially available as Augmentin®), moxifloxacin hydrochloride (commercially available as Avelox®), besifloxacin (commercially available as Besivance®), clarithromycin (commercially available as Biaxin®), ceftibuten (commercially available as Cedax®), cefuroxime axetil (commercially available as Ceftin®), cefprozil (commercially available as Cefzil®), ciprofloxacin hydrochloride (commercially available as Ciloxan® and Cipro®), clindamycin phosphate (commercially available as Cleocin T®), doxycycline hyclate (commercially available as Doryx®), dirithromycin (commercially available as Dynabac®), erythromycin (commercially available as E.E.S.®, E-Mycin®, Eryc®, Ery-Tab®, Erythrocin®, and PCE®), erythromycin topical (commercially available as A/T/S®, Erycette®, T-Stat®), gemifloxacin (commercially available as Factive®), ofloxacin (commercially known as Ocuflox®, Floxin®), telithromycin (commercially available as Ketek®), lomefloxacin hydrochloride (commercially available as Maxaquin®), minocycline hydrochloride (commercially available as Minocin®), fosfomycin tromethamine (commercially available as Monurol®), penicillin with potassium (commercially available as Penicillin VK®, Veetids®), trimethoprim (commercially available as Primsol®), ciprofloxacin hydrochloride (commercially available as Proquin XR®), rifampin, isoniazid and pyrazinamide (commercially available as Rifater®), cefditoren (commercially available as Spectracef®), cefixime (commercially available as Suprax®), tetracycline (commercially available as Achromycin V® and Sumycin®), tobramycin (commercially available as Tobrex®), rifaximin (commercially available as Xifaxan®), azithromycin (commercially available as Zithromax®), azithromycin suspension (commercially available as Zmax®), linezolid (commercially available as Zyvox®), benzoyl peroxide and clindamycin (commercially available as BenzaClin®), erythromycin and benzoyl peroxide (commercially available as Benzamycin®), dexamethasone (commercially available as Ozurdex®), ciprofloxacin and dexamethasone (commercially available as Ciprodex®), polymyxin B sulfate/neomycin sulfate/hydrocortisone (commercially available as Cortisporin®), colistin sulfate/neomycin sulfate/hydrocortisone acetate/thonzonium bromide (commercially available as Cortisporin-TC Otic®), cephalexin hydrochloride (commercially available as Keflex®), cefdinir (commercially available as Omnicef®), and gatifloxacin (commercially available as Zymar®).

Other useful actives include cancer treatment medications, including cyclophosphamide (commercially available as Cytoxan®), methotrexate (commercially available as Rheumatrex® and Trexal®), tamoxifen citrate (commercially available as Nolvadex®), bevacizumab (commercially available as Avastin®), everolimus (commercially available as Afinitor®), pazopanib (commercially available as Votrient®), and anastrozole (commercially available as Arimidex®); leukemia treatment, such as ofatumumab (commercially available as Arzerra®); anti-thrombotic drugs, such as antithrombin recombinant lyophilized powder (commercially available as Atryn®), prasugrel (commercially available as Efient®); anti-coagulants, such as aspirin with extended-release dipyridamole (commercially available as Aggrenox®), warfarin sodium (commercially available as Coumadin®), dipyridamole (commercially available as Persantine®), dalteparin (commercially available as Fragmin®), danaparoid (commercially available as Orgaran®), enoxaparin (commercially available as Lovenox®), heparin (commercially available as Hep-Lock, Hep-Pak, Hep-Pak CVC, Heparin Lock Flush), tinzaparin (commercially available as Innohep®), and clopidogrel bisulfate (commercially available as Plavix®); antiemetics, such as granisetron hydrochloride (commercially available as Kytril®) and nabilone (commercially available as Cesamet®), trimethobenzamide hydrochloride (commercially available as Tigan®), and ondansetron hydrochloride (commercially available as Zofran®); anti-fungal treatment, such as ketoconazole (commercially available as Nizoral®), posaconazole (commercially available as Noxafil®), ciclopirox (commercially available as Penlac®), griseofulvin (commercially available as Gris-PEG®), oxiconazole nitrate (commercially available as Oxistat®), fluconazole (commercially available as Diflucan®), sertaconazole nitrate (commercially available as Ertaczo®), terbinafine hydrochloride (commercially available as Lamisil®), ciclopirox (commercially available as Loprox®), nystatin/triamcinolone acetonide (commercially available as Mycolog-II®), econazole nitrate (commercially available as Spectazole®), itraconazole (commercially available as Sporanox®), and terconazole (commercially available as Terazol®).

Active agents may further include anti-inflammatory medications, such as hydroxychloroquine sulfate (commercially available as Plaquenil®), fluticasone propionate (commercially available as Cutivate®), canakinumab (commercially available as Llaris®), amcinonide (commercially available as Cyclocort®), methylprednisolone (commercially available as Medrol®), budesonide (commercially available as Entocort EC®), anakinra (commercially available as Kineret®), diflorasone diacetate (commercially available as Psorcon®), and etanercept (commercially available as Enbrel®); antispasmodic medication, such as phenobarbital/hyoscyamine sulfate/atropine sulfate/scopolamine hydrobromide (commercially available as Donnatal®); antiviral treatment, such as oseltamivir phosphate (commercially available as Tamiflu®); anti-parasites medication, including tinidazole (commercially available as Tindamax®); appetite treatment mediations, such as megestrol acetate (commercially available as Megace ES®), phentermine hydrochloride (commercially available as Adipex-P®), and diethylpropion hydrochloride (commercially available as Tenuate®); arthritis medications, including leflunomide (commercially available as Arava®), certolizumab pegol (commercially available as Cimzia®), diclofenac sodium (commercially available as Pennsaid®), golimumab (commercially available as Simponi®), and tocilizumab (commercially available as Actemra®); bladder control medication, such as trospium chloride (commercially available as Sanctura®), desmopressin acetate (commercially available as DDAVP®), tolterodine tartrate (commercially available as Detrol®), oxybutynin chloride (commercially available as Ditropan® or Gelnique®), darifenacin (commercially available as Enablex®), and solifenacin succinate (commercially available as VESIcare®); blood vessel constrictors, such as methylergonovine maleate (commercially available as Methergine®); plasma uric managers, such as rasburicase (commercially available as Elitek®); iron deficiency anemia medications, such as ferumoxytol (commercially available as Feraheme®); lymphoma medications, such as pralatrexate (commercially available as Folotyn®), romidepsin (commercially available as Isodax®); malaria medication, such as artemether/lumefantrine (commercially available as Coartem®); hyponatremia medication, such as tolvatpan (commercially available as Samsca®); medication for treatment of von Willebrand disease (commercially available as Wilate®); anti-hypertension medications, such as treprostinil (commercially available as Tyvaso®), tadalafil (commercially available as Adcirca®); cholesterol lowering medication, including paricalcitol (commercially available as Altocor®), pitavastatin (commercially available as Livalo®), lovastatin, niacin (commercially available as Advicor®), colestipol hydrochloride (commercially available as Colestid®), rosuvastatin calcium (commercially available as Crestor®), fluvastatin sodium (commercially available as Lescol®), atorvastatin calcium (commercially available as Lipitor®), lovastatin (commercially available as Mevacor®), niacin (commercially available as Niaspan®), pravastatin sodium (commercially available as Pravachol®), pavastatin sodium with buffered aspirin (commercially available as Pravigard PAC®), cholestyramine (commercially available as Questran®), simvastatin and niacin (commercially available as Simcor®), atenolol, chlorthalidone (commercially available as Tenoretic®), atenolol (commercially available as Tenormin®), fenofibrate (commercially available as Tricor®), fenofibrate (commercially available as Triglide®), ezetimibe/simvastatin (commercially available as Vytorin®), colesevelam (commercially available as WelChol®), bisoprolol fumarate (commercially available as Zebeta®), ezetimibe (commercially available as Zetia®), bisoprolol fumarate/hydrochlorothiazide (commercially available as Ziac®), and simvastatin (commercially available as Zocor®).

The pharmaceutical actives included herein may also include chronic kidney disease medication, such as paricalcitol (commercially available as Zemplar®); contraceptive agents, including etonogestrel (commercially available as Implanon®), norethindrone acetate, ethinyl estradiol (commercially available as Loestrin 24 FE®), ethinyl estradiol, norelgestromin (commercially available as Ortho Evra®), levonorgestrel (commercially available as Plan B®), levonorgestrel and ethinyl estradiol (commercially available as Preven®), levonorgestrel, ethinyl estradiol (commercially available as Seasonique®), and medroxyprogesterone acetate (commercially available as Depo-Provera®); COPD medication, such as arformoterol tartrate (commercially available as Brovana®) and ipratropium bromide, albuterol sulfate (commercially available as Combivent®); cough suppressants, including benzonatate (commercially available as Tessalon®), guaifenesin, codeine phosphate (commercially available as Tussi-Organidin NR®), and acetaminophen, codeine phosphate (commercially available as Tylenol with Codeine®); medication for the treatment of diabetes, including pioglitazone hydrochloride, metformin hydrochloride (commercially available as ACTOplus Met®), bromocriptine mesylate (commercially available as Cycloset®), liraglutide (commercially available as Victoza®), saxagliptin (commercially available as Onglyza®), pioglitazone hydrochloride (commercially available as Actos®), glimepiride (commercially available as Amaryl®), rosiglitazone maleate, metformin hydrochloride (commercially available as Avandamet®), rosiglitazone maleate (commercially available as Avandaryl®), rosiglitazone maleate (commercially available as Avandia®), exenatide (commercially available as Byetta®), exenatide (commercially available as Bydureon®), chlorpropamide (commercially available as Diabinese®), pioglitazone hydrochloride, glimepiride (commercially available as Duetact®), metformin hydrochloride (commercially available as Glucophage®), glipizide (commercially available as Glucotrol®), glyburide, metformin (commercially available as Glucovance® and Fortamet®), metformin hydrochloride (commercially available as Glumetza®), sitagliptin (commercially available as Januvia®), detemir (commercially available as Levemir®), glipizide, metformin hydrochloride (commercially available as Metaglip®), glyburide (commercially available as Micronase®), repaglinide (commercially available as Prandin®), acarbose (commercially available as Precose®), nateglinide (commercially available as Starlix®), pramlintide acetate (commercially available as Symlin®), canagliflozin (commercially available as Invokana®), linagliptin (commercially available as Tradjenta®), dapagliflozin (commercially available as Farxiga®), insulin glargine (commercially available as Lantus® or Toujeo®), insulin aspart (commercially available as Novolog®), insulin lispro, empagliflozin (commercially available as Jardiance®), and tolazamide (commercially available as Tolinase®).

Other useful actives of the present invention may include digestive agents, such as sulfasalazine (commercially available as Azulfidine®), rabeprazole sodium (commercially available as AcipHex®), lubiprostone (commercially available as Amitiza®), dicyclomine hydrochloride (commercially available as Bentyl®), sucralfate (commercially available as Carafate®), lactulose (commercially available as Chronulac®), docusate (commercially available as Colace®), balsalazide disodium (commercially available as Colazal®), losartan potassium (commercially available as Cozaar®), olsalazine sodium (commercially available as Dipentum®), chlordiazepoxide hydrochloride, clidinium bromide (commercially available as Librax®), esomeprazole magnesium (commercially available as Nexium®), famotidine (commercially available as Pepcid®), lansoprazole (commercially available as Prevacid®), lansoprazole and naproxen (commercially available as Prevacid NapraPAC®), amoxicillin/clarithromycin/lansoprazole (commercially available as Prevpac®), omeprazole (commercially available as Prilosec®), pantoprazole sodium (commercially available as Protonix®), metoclopramide hydrochloride (commercially available as Reglan® or Metozolv®), cimetidine (commercially available as Tagamet®), ranitidine hydrochloride (commercially available as Zantac®), and omeprazole, sodium bicarbonate (commercially available as Zegerid®); diuretics, including spironolactone, hydrochlorothiazide (commercially available as Aldactazide®), spironolactone (commercially available as Aldactone®), bumetanide (commercially available as Bumex®), torsemide (commercially available as Demadex®), chlorothiazide (commercially available as Diuril®), furosemide (commercially available as Lasix®), metolazone (commercially available as Zaroxolyn®), and hydrochlorothiazide, triamterene (commercially available as Dyazide®).

Actives useful herein may also include treatment for emphysema, such as tiotropium bromide (commercially available as Spiriva®); fibromyalgia medication, such as milnacipran hydrochloride (commercially available as Savella®); medication for the treatment of gout, such as colchicine (commercially available as Colcrys®), and febuxostat (commercially available as Uloric®); enema treatments, including aminosalicylic acid (commercially available as Mesalamine® and Rowasa®); epilepsy medications, including valproic acid (commercially available as Depakene®), felbamate (commercially available as Felbatol®), lamotrigine (commercially available as Lamictal®), primidone (commercially available as Mysoline®), oxcarbazepine (commercially available as Trileptal®), zonisamide (commercially available as Zonegran®), levetiracetam (commercially available as Keppra®), and phenytoin sodium (commercially available as Dilantin®).

Erectile dysfunction therapies useful herein include, but are not limited to, drugs for facilitating blood flow to the penis, and for effecting autonomic nervous activities, such as increasing parasympathetic (cholinergic) and decreasing sympathetic (adrenersic) activities. Useful agents for treatment of erectile dysfunction include, for example, those agents available as alprostadil (commercially available as Caverject®), tadalafil (commercially available as Cialis®), vardenafil (commercially available as Levitra®), apomorphine (commercially available as Uprima®), yohimbine hydrochloride (commercially available as Aphrodyne®, Yocon®), and sildenafil citrate (commercially available as Viagra®).

Actives useful herein may further include eye medications and treatment, such as dipivefrin hydrochloride (commercially available as Propine®), valganciclovir (commercially available as Valcyte®), ganciclovir ophthalmic gel (commercially available as Zirgan®); bepotastine besilate (commercially available as Bepreve®), besifloxacin (commercially available as Besivance®), bromfenac (commercially available as Xibrom®), fluorometholone (commercially available as FML®), pilocarpine hydrochloride (commercially available as Pilocar®), cyclosporine (commercially available as Restasis®), brimonidine tartrate (commercially available as Alphagan P®), dorzolamide hydrochloride/timolol maleate (commercially available as Cosopt®), bimatoprost (commercially available as Lumigan®), timolol maleate (available as Timoptic®), travoprost (commercially available as Travatan®), latanoprost (commercially available as Xalatan®), echothiophate iodide (commercially available as Phospholine Iodide®), and ranibizumab (commercially available as Lucentis®); fluid controllers, such as acetazolamide (commercially available as Diamox®); gallstone medications, including ursodiol (commercially available as Actigall®); medication for the treatment of gingivitis, including chlorhexidine gluconate (commercially available as Peridex®); headache medications, including butalbital/codeine phosphate/aspirin/caffeine (commercially available as Fiornal® with Codeine), naratriptan hydrochloride (commercially available as Amerge®), almotriptan (commercially available as Axert®), ergotamine tartrate/caffeine (commercially available as Cafergot®), butalbital/acetaminophen/caffeine (commercially available as Fioricet®), butalbital/aspirin/caffeine (commercially available as Fiorinal®), frovatriptan succinate (commercially available as Frova®), rizatriptan benzoate (commercially available as Maxalt®), isometheptene mucate/dichloralphenazone/acetaminophen (commercially available as Midrin®), dihydroergotamine mesylate (commercially available as Migranal®), eletriptan hydrobromide (commercially available as Relpax®), and zolmitriptan (commercially available as Zomig®); influenza medication, such as haemophilus b conjugate vaccine; tetanus toxoid conjugate (commercially available as Hiberix®); and heart treatments, including quinidine sulfate, isosorbide dinitrate/hydralazine hydrochloride (commercially available as BiDil®), digoxin (commercially available as Lanoxin®), flecainide acetate (commercially available as Tambocor®), mexiletine hydrochloride (commercially available as Mexitil®), disopyramide phosphate (commercially available as Norpace®), procainamide hydrochloride (commercially available as Procanbid®), and propafenone (commercially available as Rythmol®).

Other useful actives include hepatitis treatments, including entecavir (commercially available as Baraclude®), hepatitis B immune globulin (commercially available as HepaGam B®), and copegus/rebetol/ribasphere/vilona/virazole (commercially available as Ribavirin®); herpes treatments, including valacyclovir hydrochloride (commercially available as Valtrex®), penciclovir (commercially available as Denavir®), acyclovir (commercially available as Zovirax®), and famciclovir (commercially available as Famvir®); treatment for high blood pressure, including enalaprilat (available as Vasotec®), captopril (available as Capoten®) and lisinopril (available as Zestril®), verapamil hydrochloride (available as Calan®), ramipril (commercially available as Altace®), olmesartan medoxomil (commercially available as Benicar®), amlodipine/atorvastatin (commercially available as Caduet®), nicardipine hydrochloride (commercially available as Cardene®), diltiazem hydrochloride (commercially available as Cardizem®), quinapril hydrochloride (commercially available as Accupril®), quinapril hydrochloride/hydrochlorothiazide (commercially available as Accuretic®), perindopril erbumine (commercially available as Aceon®), candesartan cilexetil (commercially available as Atacand®), candesartan cilexetil/hydrochlorothiazide (commercially available as Atacand HCT®), irbesartan/hydrochlorothiazide (commercially available as Avalide®), irbesartan (commercially available as Avapro®), amlodipine besylate/olmesartan medoxomil (commercially available as Azor®), levobunolol hydrochloride (commercially available as Betagan®), betaxolol hydrochloride (commercially available as Betoptic®), nebivolol (commercially available as Bystolic®), captopril/hydrochlorothiazide (commercially available as Capozide®), doxazosin mesylate (commercially available as Cardura®), clonidine hydrochloride (commercially available as Catapres®), carvedilol (commercially available as Coreg®), nadolol (commercially available as Corgard®), nadolol/bendroflumethiazide (commercially available as Corzide®), valsartan (commercially available as Diovan®), isradipine (commercially available as DynaCirc®), Guanabenz acetate. (commercially available as Wytensin®), Guanfacine hydrochloride (commercially available as Tenex® or Intuniv®), losartan potassium/hydrochlorothiazide (commercially available as Hyzaar®), propranolol hydrochloride (commercially available as Indera®), propranolol hydrochloride/hydrochlorothiazide (commercially available as Inderide®), eplerenone (commercially available as Inspra®), ambrisentan (commercially available as Letairis®), enalapril maleate/felodipine (commercially available as Lexxel®), metoprolol tartrate (commercially available as Lopressor®), benazepril hydrochloride (commercially available as Lotensin®), benazepril hydrochloride/hydrochlorothiazide (commercially available as Lotensin HCT®), amlodipine/benazepril hydrochloride (commercially available as Lotrel®), indapamide (commercially available as Lozol®), trandolapril (commercially available as Mavik®), telmisartan (commercially available as Micardis®), telmisartan/hydrochlorothiazide (commercially available as Micardis HCT®), prazosin hydrochloride (commercially available as Minipress®), amiloride, hydrochlorothiazide (commercially available as Moduretic®), fosinopril sodium (commercially available as ZZXT Monopril®), fosinopril sodium/hydrochlorothiazide (commercially available as Monopril-HCT®), pindolol (commercially available as Visken®), felodipine (commercially available as Plendil®), sildenafil citrate (commercially available as Revatio®), Nisoldipine (commercially available as Sular®), trandolapril/verapamil hydrochloride (commercially available as Tarka®), aliskiren (commercially available as Tekturna®), eprosartan mesylate (commercially available as Teveten®), eprosartan mesylate/hydrochlorothiazide (commercially available as Teveten HCT®), moexipril hydrochloride/hydrochlorothiazide (commercially available as Uniretic®), moexipril hydrochloride (commercially available as Univasc®), enalapril maleate/hydrochlorothiazide (commercially available as Vaseretic®), and lisinopril/hydrochlorothiazide (commercially available as Zestoretic®).

The dosage forms of the present disclosure, such as the films of the present disclosure, may include actives useful in the medication for the treatment of HIV/AIDS, such as amprenavir (commercially available as Agenerase®), tipranavir (commercially available as Aptivus®), efavirenz/emtricitabine/tenofovir (commercially available as Atripla®), lamivudine/zidovudine (commercially available as Combivir®), indinavir sulfate (commercially available as Crixivan®), lamivudine (commercially available as Epivir®), saquinavir (commercially available as Fortovase®), zalcitabine (commercially available as Hivid®), lopinavir/ritonavir (commercially available as Kaletra®), fosamprenavir calcium (commercially available as Lexiva®), ritonavir (commercially available as Norvir®), zidovudine (commercially available as Retrovir®), atazanavir sulfate (commercially available as Reyataz®), efavirenz (commercially available as Sustiva®), abacavir/lamivudine/zidovudine (commercially available as Trizivir®), didanosine (commercially available as Videx®), nelfinavir mesylate (commercially available as Viracept®), nevirapine (commercially available as Viramune®), tenofovir disoproxil fumarate (commercially available as Viread®), stavudine (commercially available as Zerit®), and abacavir sulfate (commercially available as Ziagen®); homocysteiene removers, including betaine anhydrous (commercially available as Cystadane®); medications, such as insulin (commercially available as Apidra®, Humalog®, Humulin®, Iletin®, Tresiba®, and Novolin®); and HPV treatment, such as Human papillomavirus vaccine (commercially available as Gardasil®) or human papillomavirus bivalent (commercially available as Cervarix®); immunosuppressants, including cyclosporine (commercially available as Gengraf®, Neoral®, Sandimmune®, and Apo-Cyclosporine®).

Actives useful in the present disclosure may further include prolactin inhibitors, such as bromocriptine mesylate (commercially available as Parlodel®); medications for aiding in stress tests, such as regadenoson (commercially available as Lexiscan®); baldness medication, including finasteride (commercially available as Propecia® and Proscar®); pancreatitis treatment, such as gemfibrozil (commercially available as Lopid®); hormone medications, such as norethindrone acetate/ethinyl estradiol (commercially available as femHRT®), goserelin acetate (commercially available as Zoladex®), progesterone gel (commercially available as Prochieve®), progesterone (commercially available as Prometrium®), calcitonin-salmon (commercially available as Miacalcin®), calcitriol (commercially available as Rocaltrol®), synthroid (commercially available as Levothroid®, Levoxyl®, Unithroid®), testosterone (commercially available as Testopel®, Androderm®, Testoderm®, and AndroGel®); menopause medication, such as estradiol/norethindrone acetate (commercially available as Activella®), drospirenone/estradiol (commercially available as Angeliq®), estradiol/levonorgestrel (commercially available as Climara Pro®), estradiol/norethindrone acetate (commercially available as CombiPatch®), estradiol (commercially available as Estrasorb®, Vagifem® and EstroGel®), esterified estrogens and methyltestosterone (commercially available as Estratest®), estrogen (commercially available as Alora®, Climara®, Esclim®, Estraderm®, Vivelle®, Vivelle-Dot®), estropipate (commercially available as Ogen®), conjugated estrogens (commercially available as Premarin®), and medroxyprogesterone acetate (commercially available as Provera®); menstrual medications, including leuprolide acetate (commercially available as Lupron Depot), tranexamic acid (commercially available as Lysteda®), and norethindrone acetate (commercially available as Aygestin®); and muscle relaxants, including cyclobenzaprine hydrochloride (commercially available as Flexeril®), tizanidine (commercially available as Zanaflex®), and hyoscyamine sulfate (commercially available as Levsin®).

Actives useful herein may also include osteoporosis medications, including ibrandronate sodium (commercially available as Boniva®), risedronate (commercially available as Actonel®), raloxifene hydrochloride (commercially available as Evista®, Fortical®), and alendronate sodium (commercially available as Fosamax®); ovulation enhancers, including clomiphene citrate (commercially available as Serophene®, Clomid®, Serophene®); Paget's disease treatment, such as etidronate disodium (commercially available as Didronel®); pancreatic enzyme deficiency medications, such as pancrelipase (commercially available as Pancrease® or Zenpep®); medication for the treatment of Parkinson's disease, such as pramipexole dihydrochloride (commercially available as Mirapex®), ropinirole hydrochloride (commercially available as Requip®), carbidopa/levodopa (commercially available as Sinemet CR®), carbidopa/levodopa/entacapone (commercially available as Stalevo®), selegiline hydrochloride (commercially available as Zelapar®), rasagiline (commercially available as Azilect®), entacapone (commercially available as Comtan®), and selegiline hydrochloride (commercially available as Eldepryl®); multiple sclerosis medication, such as dalfampridine (commercially available as Ampyra®) and interferon beta-I b (commercially available as Extavia®); prostate medication, including flutamide (commercially available as Eulexin®), nilutamide (commercially available as Nilandron®), dutasteride (commercially available as Avodart®), tamsulosin hydrochloride (commercially available as Flomax®), terazosin hydrochloride (commercially available as Hytrin®), and alfuzosin hydrochloride (commercially available as UroXatral®).

The dosage forms of the present disclosure, such as the films of the present disclosure, may further include psychiatric medications, including alprazolam (available as Niravam®, Xanax®), clozopin (available as Clozaril®), haloperidol (available as Haldol®), fluoxetine hydrochloride (available as Prozac®), sertraline hydrochloride (available as Zoloft®), asenapine (commercially available as Saphris®), iloperidone (commercially available as Fanapt®), paroxtine hydrochloride (available as Paxil®), aripiprazole (commercially available as Abilify®), guanfacine (commercially available as Intuniv®), Amphetamines and methamphetamines (commercially available as Adderall® and Desoxyn®), clomipramine hydrochloride (commercially available as Anafranil®), Buspirone hydrochloride (commercially available as BuSpar®), citalopram hydrobromide (commercially available as Celexa®), duloxetine hydrochloride (commercially available as Cymbalta®), methylphenidate (commercially available as Ritalin, Daytrana®), divalproex sodium (Valproic acid) (commercially available as Depakote®), dextroamphetamine sulfate (commercially available as Dexedrine®), venlafaxine hydrochloride (commercially available as Effexor®), selegiline (commercially available as Emsam®), carbamazepine (commercially available as Equetro®), lithium carbonate (commercially available as Eskalith®), fluvoxamine maleate/dexmethylphenidate hydrochloride (commercially available as Focalin®), ziprasidone hydrochloride (commercially available as Geodon®), ergoloid mesylates (commercially available as Hydergine®), escitalopram oxalate (commercially available as Lexapro®), chlordiazepoxide (commercially available as Librium®), molindone hydrochloride (commercially available as Moban®), phenelzine sulfate (commercially available as Nardil®), thiothixene (commercially available as Navane®), desipramine hydrochloride (commercially available as Norpramin®), benzodiazepines (such as those available as Oxazepam®), nortriptyline hydrochloride (commercially available as Pamelor®), tranylcypromine sulfate (commercially available as Parnate®), prochlorperazine, mirtazapine (commercially available as Remeron®), risperidone (commercially available as Risperdal®), quetiapine fumarate (commercially available as Seroquel®), doxepin hydrochloride (commercially available as Sinequan®), atomoxetine hydrochloride (commercially available as Strattera®), trimipramine maleate (commercially available as Surmontil®), olanzapine/fluoxetine hydrochloride (commercially available as Symbyax®), imipramine hydrochloride (commercially available as Tofranil®), protriptyline hydrochloride (commercially available as Vivactil®), bupropion hydrochloride (commercially available as Wellbutrin®, Wellbutrin SR®, and Wellbutrin XR®), and olanzapine (commercially available as Zyprexa®).

Actives useful herein may also include uric acid reduction treatment, including allopurinol (commercially available as Zyloprim®); seizure medications, including gabapentin (commercially available as Neurontin®), ethotoin (commercially available as Peganone®), vigabatrin (commercially available as Sabril®), and topiramate (commercially available as Topamax®); treatment for shingles, such as zoster vaccine live (commercially available as Zostavax®); skin care medications, including calcipotriene (commercially available as Dovonex®), ustekinumab (commercially available as Stelara®), televancin (commercially available as Vibativ®), isotretinoin (commercially available as Accutane®), hydrocortisone/iodoquinol (commercially available as Alcortin®), sulfacetamide sodium/sulfur (commercially available as Avar®), azelaic acid (commercially available as Azelex®, Finacea®), benzoyl peroxide (commercially available as Desquam-E®), adapalene (commercially available as Differin®), fluorouracil (commercially available as Efudex®), pimecrolimus (commercially available as Elidel®), topical erythromycin (commercially available as A/T/S®, Erycette®, T-Stat®), hydrocortisone (commercially available as Cetacort®, Hytone®, Nutracort®), metronidazole (commercially available as MetroGel®), doxycycline (commercially available as Oracea®), tretinoin (commercially available as Retin-A® and Renova®), mequinol/tretinoin (commercially available as Solage®), acitretin (commercially available as Soriatane®), calcipotriene hydrate/betamethasone dipropionate (commercially available as Taclonex®), tazarotene (commercially available as Tazorac®), fluocinonide (commercially available as Vanos®), desonide (commercially available as Verdeso®), miconazole nitrate/Zinc oxide (commercially available as Vusion®), ketoconazole (commercially available as Xolegel®), and efalizumab (commercially available as Raptiva®).

Other actives useful herein may include Sleep disorder medications, including zaleplon (available as Sonata®), eszopiclone (available as Lunesta®), zolpidem tartrate (commercially available as Ambien®, Ambien CR®, Edluar®), lorazepam (commercially available as Ativan®), flurazepam hydrochloride (commercially available as Dalmane®), triazolam (commercially available as Halcion®), clonazepam (commercially available as Klonopin®), barbituates, such as Phenobarbital®), Modafinil (commercially available as Provigil®), temazepam (commercially available as Restoril®), ramelteon (commercially available as Rozerem®), clorazepate dipotassium (commercially available as Tranxene®), diazepam (commercially available as Valium®), quazepam (commercially available as Doral®), and estazolam (commercially available as ProSom®); smoking cessation medications, such as varenicline (commercially available as Chantix®), nicotine, such as Nicotrol®, and bupropion hydrochloride (commercially available as Zyban®); and steroids, including alclometasone dipropionate (commercially available as Aclovate®), betamethasone dipropionate (commercially available as Diprolene®), mometasone furoate (commercially available as Elocon®), fluticasone (commercially available as Flonase®, Flovent®, Flovent Diskus®, Flovent Rotadisk®), fluocinonide (commercially available as Lidex®), mometasone furoate monohydrate (commercially available as Nasonex®), desoximetasone (commercially available as Topicort®), clotrimazole/betamethasone dipropionate (commercially available as Lotrisone®), prednisolone acetate (commercially available as Pred Forte®, Prednisone®, Budesonide Pulmicort®, Rhinocort Aqua®), prednisolone sodium phosphate (commercially available as Pediapred®), desonide (commercially available as Tridesilon®), and halobetasol propionate (commercially available as Ultravate®).

The dosage forms of the present invention, such as the films of the present invention, may further include actives useful for thyroid disease treatment, such as hormones TC and TD (commercially available as Armour Thyroid®); potassium deficiency treatment, including potassium chloride (commercially available as Micro-K®); triglycerides regulators, including omega-3-acid ethyl esters (commercially available as Omacor®); urinary medication, such as phenazopyridine hydrochloride (commercially available as Pyridium®) and methenamine, methylene blue/phenyl salicylate/benzoic acid/atropine sulfate/hyoscyamine (commercially available as Urised®); prenatal vitamins (commercially available as Advanced Natalcare®, Materna®, Natalins®, Prenate Advance®); weight control medication, including orlistat (commercially available as Xenical®) and sibutramine hydrochloride (commercially available as Meridia®).

The popular H2-antagonists which are contemplated for use herein include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as, but not limited to, plant pollens from grasses, trees, or ragweed; animal danders, which are tiny scales shed from the skin and hair of cats and other furred animals; insects, such as house dust mites, bees, and wasps; and drugs, such as penicillin.

Examples of specific actives include but are not limited to 16-alpha fluorocstradiol, 16-alpha-gitoxin, 16-epiestriol, 17 alpha dihydroequilenin, 17 alpha estradiol, 17 beta estradiol, 17 hydroxy progesterone, lalpha-hydroxyvitamin D2,1-dodecpyrrolidinone, 20-epi-1,25 dihydroxyvitamin D3, 22-oxacalcitriol, 2CVV, 2′-nor-cGMP, 3-isobutyl GABA, 5-ethynyluracil, 6-FUDCA, 7-methoxytacrine, Abamectin, abanoquil, abecarnil, abiraterone, Ablukast, Ablukast Sodium, Acadesine, acamprosate, Acarbose, Acebutolol, Acecainide Hydrochloride, Aceclidine, aceclofenae, Acedapsone, Aceglutamide Aluminum, Acemannan, Acetaminophen, Acetazolamide, Acetohexamide, Acetohydroxamic Acid, acetomepregenol, Acetophenazine Maleate, Acetosulfone Sodium, Acetylcholine Chloride, Acetylcysteine, acetyl-L-carnitine, acetylmethadol, Acifran, acipimox, acitemate, Acitretin, Acivicin, Aclarubicin, aclatonium, Acodazole Hydrochloride, aconiazide, Acrisorcin, Acrivastine, Acronine, Actisomide, Actodigin, Acyclovir, acylfulvene, adafenoxate, adapalene, Adapalene, adatanserin, Adatanserin Hydrochloride, adecypenol, adecypenol, Adefovir, adelmidrol, ademetionine, Adenosine, Adinazolam, Adipheinine Hydrochloride, adiposin, Adozelesin, adrafinil, Adrenalone, airbutamine, alacepril, Alamecin, Alanine, Alaproclate, alaptide, Albendazole, albolabrin, Albuterol, Albutoin, Alclofenae, Alclometasone Dipropionate, Alcloxa, aldecalmycin, Aldesleukin, Aldioxa, Alendronate Sodium, alendronic acid, alentemol, Alentemol Hydrobromide, Aletamine Hydrochloride, Aleuronium Chloride, Alexidine, alfacalcidol, Alfentanil Hydrochloride, alfuzosin, Algestone Acetonide, alglucerase, Aliflurane, alinastine, Alipamide, Allantoin, Allobarbital, Allopurinol, ALL-TK antagonists, Alogliptin, Alonimid, alosetron, Alosetron Hydrochloride, Alovudine, Alpertine, Alpha Amylase, alpha idosone, Alpidem, Alprazolam, Alprenolol Hydrochloride, Alprenoxime Hydrochloride, Alprostadil, Alrestatin Sodium, Altanserin Tartrate, Alteplase, Althiazide, Altretamine, altromycin B, Alverinc Citrate, Alvircept Sudotox, Amadinone Acetate, Amantadine Hydrochloride, ambamustine, Ambomycin, Ambruticin, Ambuphylline, Ambuside, Amcinafal, Amcinonide, Amdinocillin, Amdinocillin Pivoxil, Amedalin Hydrochloride, amelometasone, Ameltolide, Amesergide, Ametantrone Acetate, amezinium metilsulfate, amfebutamone, Amfenac Sodium, Amflutizole, Amicycline, Amidephrine Mesylate, amidox, Amifloxacin, amifostine, Amikacin, Amiloride Hydrochloride, Aminacrine Hydrochloride, Aminobenzoate Potassium, Aminobenzoate Sodium, Aminocaproic Acid, Aminoglutethimide, Aminohippurate Sodium, aminolevulinic acid, Aminophylline, A minorex, Aminosalicylate sodium, Aminosalicylic acid, Amiodarone, Amiprilose Hydrochloride, Amiquinsin Hydrochloride, amisulpride, Amitraz, Amitriptyline Hydrochloride, Amlexanox, amlodipine, Amobarbital Sodium, Amodiaquine, Amodiaquine Hydrochloride, Amorolfine, Amoxapine, Amoxicillin, Amphecloral, Amphetamine Sulfate, Amphomycin, Amphotericin B, Ampicillin, ampiroxicam, Ampyzine Sulfate, Amquinate, Amrinone, amrinone, amrubicin, Amsacrine, amylin, amythiamicin, Anagestone Acetate, anagrelide, Anakinra, ananain, anaritide, Anaritide Acetate, Anastrozole, Anazolene Sodium, Ancrod, andrographolide, Androstenedione, angiogenesis inhibitors, Angiotensin Amide, Anidoxime, Anileridine, Anilopam Hydrochloride, Aniracetam, Anirolac, Anisotropine Methylbromide, Anistreplase, Anitrazafen, anordrin, antagonist D, antagonist G, antarelix, Antazoline Phosphate, Anthelmycin, Anthralin, Anthramycin, antiandrogen, Acedapsone, Felbamate, antiestrogen, antineoplaston, Antipyrine, antisense oligonucleotides, apadoline, apafant, Apalcillin Sodium, apaxifylline, Apazone, aphidicolin glycinate, Apixifylline, Apomorphine Hydrochloride, apraclonidine, Apraclonidine Hydrochloride, Apramycin, Aprindine, Aprindine Hydrochloride, aprosulate sodium, Aprotinin, Aptazapine Maleate, aptiganel, apurinic acid, apurinic acid, aranidipine, Aranotin, Arbaprostil, arbekicin, arbidol, Arbutamine Hydrochloride, Arclofenin, Ardeparin Sodium, argatroban, Arginine, Argipressin Tannate, Arildone, aripiprazol, arotinolol, Arpinocid, Arteflene, Artilide Fumarate, asimadoline, aspalatone, Asparaginase, Asparic Acid, Aspartocin, asperfuran, Aspirin, aspoxicillin, Asprelin, Astemizole, Astromicin Sulfate, asulacrine, atamestane, Atenolol, atevirdine, Atipamezole, Atiprosin Maleate, Atolide, Atorvastatin Calcium, Atosiban, Atovaquone, atpenin B, Atracurium Besylate, atrimustine, atrinositol, Atropine, Auranofin, aureobasidin A, Aurothioglucose, Avilamycin, Avoparcin, Avridine, Axid, axinastatin 1, axinastatin 2, axinastatin 3, Azabon, Azacitidinie, Azaclorzine Hydrochloride, Azaconazole, azadirachtine, Azalanstat Dihydrochloride, Azaloxan Fumarate, Azanator Maleate, Azanidazole, Azaperone, Azaribine, Azaserine, azasetron, Azatadine Maleate, Azathioprine, Azathioprine Sodium, azatoxin, azatyrosine, azelaic acid, azelastine, azelnidipine, Azepindole, Azetepa, azimilide, Azithromycin, Azlocillin, Azolimine, Azosemide, Azotomycin, Aztreonam, Azumolene Sodium, Bacampicillin Hydrochloride, baccatin III, Bacitracin, Baclofen, bacoside A, bacoside B, bactobolamine, balanol, balazipone, balhimycin, balofloxacin, balsalazide, Bambermycins, bambuterol, Bamethan Sulfate, Bamifylline Hydrochloride, Bamidazole, baohuoside 1, Barmastine, barnidipine, Basifungin, Batanopride Hydrochloride, batebulast, Batelapine Maleate, Batimastat, beauvericin, Becanthone Hydrochloride, becaplermin, becliconazole, Beclomethasone Dipropionate, befloxatone, Beinserazide, Belfosdil, Belladonna, Beloxamide, Bemesetron, Bemitradine, Bemoradan, Benapryzine Hydrochloride, Benazepril Hydrochloride, Benazeprilat, Bendacalol Mesylate, Bendazac, Bendroflumethiazide, benflumetol, benidipine, Benorterone, Benoxaprofen, Benoxaprofen, Benoxinate Hydrochloride, Benperidol, Bentazepam, Bentiromide, Benurestat, Benzbromarone, Benzethonium Chloride, Benzetimide Hydrochloride, Benzilonium Bromide, Benzindopyrine Hydrochloride, benzisoxazole, Benzocaine, benzochlorins, Benzoctamine Hydrochloride, Benzodepa, benzoidazoxan, Benzonatate, Benzoyl Peroxide, Benzoylpas Calcium, benzoylstaurosporine, Benzquinamide, Benzthiazide, benztropine, Benztropine Mesylate, Benzydamine Hydrochloride, Benzylpenicilloyl Polylysine, bepridil, Bepridil Hydrochloride, Beractant, Beraprost, Berefrine, berlafenone, bertosamil, Berythromycin, besipirdine, beta-alethine, betaclamycin B, Betamethasone, betamipron, betaxolol, Betaxolol Hydrochloride, Bethanechol Chloride, Bethanidine Sulfate, betulinic acid, bevantolol, Bevantolol Hydrochloride, Bezafibrate, bFGF inhibitor, Bialamicol Hydrochloride, Biapenem, Bicalutamide, Bicifadine Hydrochloride, Biclodil Hydrochloride, Bidisomide, bifemelane, Bifonazole, bimakalim, bimithil, Bindarit, Biniramycin, binospirone, bioxalomycin alpha2, Bipenamol Hydrochloride, Biperiden, Biphenamine Hydrochloride, biriperone, bisantrene, bisaramil, bisaziridinylspermine, bis-benzimidazole A, bis-benzimidazole B, bisnafide, Bisobrin Lactate, Bisoprolol, Bispyrithione Magsulfex, bistramide D, bistramide K, bistratene A, Bithionolate Sodium, Bitolterol Mesylate, Bivalirudin, Bizelesin, Bleomycin Sulfate, Bolandiol Dipropionate, Bolasterone, Boldenone Undecylenate, boldine, Bolenol, Bolmantalate, bopindolol, Bosentan, Boxidine, brefeldin, breflate, Brequinar Sodium, Bretazenil, Bretylium Tosylate, Brifentanil Hydrochloride, brimonidine, Brinolase, Brocresine, Brocrinat, Brofoxine, Bromadoline Maleate, Bromazepam, Bromchlorenone, Bromelains, bromfenac, Brominidione, Bromocriptine, Bromodiphenhydramine Hydrochloride, Bromoxamide, Bromperidol, Bromperidol Decanoate, Brompheniramine Maleate, Broperamole, Bropirimine, Brotizolam, Bucainide Maleate, bucindolol, Buclizine Hydrochloride, Bucromarone, Budesonide, budipine, budotitane, Buformin, Bumetamide, Bunaprolast, bunazosin, Bunolol Hydrochloride, Bupicomide, Bupivacaine Hydrochloride, Buprenorphine Hydrochloride, Bupropion Hydrochloride, Buramate, Buserelin Acetate, Buspirone Hydrochloride, Busulfan, Butabarbital, Butacetin, Butaclamol Hydrochloride, Butalbital, Butamben, Butamirate Citrate, Butaperazine, Butaprost, Butedronate Tetrasodium, butenafine, Buterizine, buthionine sulfoximine, Butikacin, Butilfenin, Butirosin Sulfate, Butixirate, butixocort propionate, Butoconazole Nitrate, Butonate, Butopamine, Butoprozine Hydrochloride, Butorphanol, Butoxamine Hydrochloride, Butriptyline Hydrochloride, Cactinomycin, Cadexomer Iodine, Caffeine, calanolide A, Calcifediol, Calcipotriene, calcipotriol, Calcitonin, Calcitriol, Calcium Undecylenate, calphostin C, Calusterone, Cambendazole, camonagrel, camptothecin derivatives, canagliflozin, canarypox IL-2, candesartan, Candicidin, candoxatril, candoxatrilat, Caniglibose, Canrenoate Potassium, Canrenone, capecitabine, Capobenate Sodium, Capobenic Acid, Capreomycin Sulfate, capromab, capsaicin, Captopril, Capuride, Caracemide, Carbachol, Carbadox, Carbamazepine, Carbamide Peroxide, Carbantel Lauryl Sulfate, Carbaspirin Calcium, Carbazeran, carbazomycin C, Carbenicillin Potassium, Carbenoxolone Sodium, Carbetimer, carbetocin, Carbidopa, Carbidopa-Levodopa, Carbinoxamine Maleate, Carbiphene Hydrochloride, Carbocloral, Carbocysteine, Carbol-Fuchsin, Carboplatin, Carboprost, carbovir, carboxamide-amino-triazo-le, carboxyamidotriazole, carboxymethylated beta-1,3-glucan, Carbuterol Hydrochloride, CaRest M3, Carfentanil Citrate, Carisoprodol, Carmantadine, Carmustine, CARN 700, Camidazole, Caroxazone, carperitide, Carphenazine Maleate, Carprofen, Carsatrin Succinate, Cartazolate, carteolol, Carteolol Hydrochloride, cartilage derived inhibitor, Carubicin Hydrochloride, Carumonam Sodium, carvedilol, carvotroline, Carvotroline Hydrochloride, carzelesin, casein kinase inhibitors (ICOS), castanospermine, caurumonam, cebaracetam, cecropin B, Cedefingol, Cefaclor, Cefadroxil, Cefamandole, Cefaparole, Cefatrizine, Cefazaflur Sodium, Cefazolin, Cefbuperazone, cefcapene pivoxil, cefdaloxime pentexil tosilate, Cefdinir, cefditoren pivoxil, Cefepime, cefetamet, Cefetecol, cefixime, cefluprenam, Cefinenoxime Hydrochloride, Cefinetazole, cefminlox, cefodizime, Cefonicid Sodium, Cefoperazone Sodium, Ceforamide, cefoselis, Cefotaxime Sodium, Cefotetan, cefotiam, Cefoxitin, cefozopran, cefpimizole, Cefpiramide, cefpirome, cefpodoxime proxetil, cefprozil, Cefroxadine, cefsulodin, Ceftazidime, cefteram, ceftibuten, Ceftizoxime Sodium, ceftriaxone, Cefuroxime, celastrol, celikalim, celiprolol, cepacidiine A, Cephacetrile Sodium, Cephalexin, Cephaloglycin, Cephaloridine, Cephalothin Sodium, Cephapirin Sodium, Cephradine, cericlamine, cerivastatin, Ceronapril, certoparin sodium, Ceruletide, Cetaben Sodium, Cetalkonium Chloride, Cetamolol Hydrochloride, cetiedil, cetirizine, Cetophenicol, Cetraxate Hydrochloride, cetrorelix, Cetylpyridinium Chloride, Chenodiol, Chlophedianol Hydrochloride, Chloral Betaine, Chlorambucil, Chloramphenicol, Chlordantoin, Chlordiazepoxide, Chlorhexidine Gluconate, chlorins, Chlormadinone Acetate, chloroorienticin A, Chloroprocaine Hydrochloride, Chloropropamide, Chloroquine, chloroquinoxaline sulfonamide, Chlorothiazide, Chlorotrianisene, Chloroxine, Chloroxylenol, Chlorphenesin Carbamate, Chlorpheniramine Maleate, Chlorpromazine, Chlorpropamide, Chlorprothixene, Chlortetracycline Bisulfate, Chlorthalidone, Chlorzoxazone, Cholestyramine Resin, Chromonar Hydrochloride, cibenzoline, cicaprost, Ciclafrine Hydrochloride, Ciclazindol, ciclesonide, cicletanine, Ciclopirox, Cicloprofen, cicloprolol, Cidofovir, Cidoxepin Hydrochloride, Cifenline, Ciglitazone, Ciladopa Hydrochloride, cilansetron, Cilastatin Sodium, Cilazapril, cilnidipine, Cilobamine Mesylate, cilobradine, Cilofungin, cilostazol, Cimaterol, Cimetidine, cimetropium bromide, Cinalukast, Cinanserin Hydrochloride, Cinepazet Maleate, Cinflumide, Cingestol, cinitapride, Cinnamedrine, Cinnarizine, cinolazepam, Cinoxacin, Cinperene, Cinromide, Cintazone, Cintriamide, Cioteronel, Cipamfylline, Ciprefadol Succinate, Ciprocinonide, Ciprofibrate, Ciprofloxacin, ciprostene, Ciramadol, Cirolemycin, cisapride, cisatracurium besilate, Cisconazole, Cisplatin, cis-porphyrin, cistinexine, citalopram, Citenamide, citicoline, citreamicin alpha, cladribine, Clamoxyquin Hydrochloride, Clarithromycin, clausenamide, Clavulanate Potassium, Clazolam, Clazolimine, clebopride, Clemastine, Clentiazem Maleate, Clidinium Bromide, clinafloxacin, Clindamycin, Clioquinol, Clioxamide, Cliprofen, clobazam, Clobetasol Propionate, Clobetasone Butyrate, Clocortolone Acetate, Clodanolene, Clodazon Hydrochloride, clodronic acid, Clofazimine, Clofibrate, Clofilium Phosphate, Clogestone Acetate, Clomacran Phosphate, Clomegestone Acetate, Clometherone, clomethiazole, clomifene analogues, Clominorex, Clomiphene, Clomipramine Hydrochloride, Clonazepam, Clonidine, Clonitrate, Clonixeril, Clonixin, Clopamide, Clopenthixol, Cloperidone Hydrochloride, clopidogrel, Clopimozide, Clopipazan Mesylate, Clopirac, Cloprednol, Cloprostenol Sodium, Clorazepate Dipotassium, Clorethate, Clorexolone, Cloroperone Hydrochloride, Clorprenaline Hydrochloride, Clorsulon, Clortermine Hydrochloride, Closantel, Closiramine Aceturate, Clothiapine, Clothixamide Maleate Cloticasone Propionate, Clotrimazole, Cloxacillin Benzathine, Cloxyquin, Clozapine, Cocaine, Coccidioidin, Codeine, Codoxime, Colchicine, colestimide, Colestipol Hydrochloride, Colestolone, Colforsin, Colfosceril Palmitate, Colistimethate Sodium, Colistin Sulfate, collismycin A, collismycin B, Colterol Mesylate, combretastatin A4, combretastatin analogue, complestatin, conagenin, Conorphone Hydrochloride, contignasterol, contortrostatin, Cormethasone Acetate, Corticorelin Ovine Triflutate, Corticotropin, Cortisone Acetate, Cortivazol, Cortodoxone, cosalane, costatolide, Cosyntropin, cotinine, Coumadin, Coumermycin, crambescidin 816, Crilvastatin, crisnatol, Cromitrile Sodium, Cromolyn Sodium, Crotamiton, cryptophycin 8, cucumariosid, Cuprimyxin, curacin A, curdlan sulfate, curiosin, Cyclacillin, Cyclazocine, cyclazosin, cyclic HPMPC, Cyclindole, Cycliramine Maleate, Cyclizine, Cyclobendazole, cyclobenzaprine, cyclobut A, cyclobut G, cyclocapron, Cycloguanil Pamoate, Cycloheximide, cyclopentanthraquinones, Cyclopenthiazide, Cyclopentolate Hydrochloride, Cyclophenazine Hydrochloride, Cyclophosphamide, cycloplatam, Cyclopropane, Cycloserine, cyclosin, Cyclosporine, cyclothialidine, Cyclothiazide, cyclothiazomycin, Cyheptamide, cypemycin, Cypenamine Hydrochloride, Cyprazepam, Cyproheptadine Hydrochloride, Cyprolidol Hydrochloride, cyproterone, Cyproximide, Cysteamine, Cysteine Hydrochloride, Cystine, Cytarabine, Cytarabine Hydrochloride, cytarabine ocfosfate, cytochalasin B, cytolytic factor, cytostatin, Dacarbazine, dacliximab, dactimicin, Dactinomycin, daidzein, Daledalin Tosylate, dalfopristin, Dalteparin Sodium, Daltroban, Dalvastatin, danaparoid, Danazol, Dantrolene, dapagliflozin, daphlnodorin A, dapiprazole, dapitant, Dapoxetine Hydrochloride, Dapsone, Daptomycin, Darglitazone Sodium, darifenacin, darlucin A, Darodipine, darsidomine, Daunorubicin Hydrochloride, Dazadrol Maleate, Dazepinil Hydrochloride, Dazmegrel, Dazopride Fumarate, Dazoxiben Hydrochloride, Debrisoquin Sulfate, Decitabine, deferiprone, deflazacort, Dehydrocholic Acid, dehydrodidemnin B, Dehydroepiandrosterone, delapril, Delapril Hydrochloride, Delavirdine Mesylate, delequamine, delfaprazine, Delmadinone Acetate, delmopinol, delphinidin, Demecarium Bromide, Demeclocycline, Demecycline, Demoxepam, Denofungin, deoxypyridinoline, Depakote, deprodone, Deprostil, depsidomycin, deramciclane, dermatan sulfate, Desciclovir, Descinolone Acetonide, Desflurane, Desipramine Hydrochloride, desirudin, Deslanoside, deslorelin, desmopressin, desogestrel, Desonide, Desoximetasone, desoxoamiodarone, Desoxycorticosterone Acetate, detajmium bitartrate, Deterenol Hydrochloride, Detirelix Acetate, Devazepide, Dexamethasone, Dexamisole, Dexbrompheniramine Maleate, Dexchlorpheniramine Maleate, Dexclamol Hydrochloride, Dexetimide, Dexfenfluramine Hydrochloride, dexifosfamide, Deximafen, Dexivacaine, dexketoprofen, dexloxiglumide, Dexmedetomidine, Dexormaplatin, Dexoxadrol Hydrochloride, Dexpanthenol, Dexpemedolac, Dexpropranolol Hydrochloride, Dexrazoxane, dexsotalol, dextrin 2-sulphate, Dextroamphetamine, Dextromethorphan, Dextrorphan Hydrochloride, Dextrothyroxine Sodium, dexverapamil, Dezaguanine, dezinamide, dezocine, Diacetolol Hydrochloride, Diamocaine Cyclamate, Diapamide, Diatrizoate Meglumine, Diatrizoic Acid, Diaveridine, Diazepam, Diaziquone, Diazoxide, Dibenzepin Hydrochloride, Dibenzothiophene, Dibucaine, Dichliorvos, Dichloralphenazone, Dichlorphenamide, Dicirenone, Diclofenac Sodium, Dicloxacillin, dicranin, Dicumarol, Dicyclomine Hydrochloride, Didanosine, didemnin B, didox, Dienestrol, dienogest, Diethylcarbamazine Citrate, diethylhomospermine, diethylnorspermine, Diethylpropion Hydrochloride, Diethylstilbestrol, Difenoximide Hydrochloride, Difenoxin, Diflorasone Diacetate, Difloxacin Hydrochloride, Difluanine Hydrochloride, Diflucortolone, Diflumidone Sodium, Diflunisal, Difluprednate, Diftalone, Digitalis, Digitoxin, Digoxin, Dihexyverine Hydrochloride, dihydrexidine, dihydro-5-azacytidine, Dihydrocodeine Bitartrate, Dihydroergotamine Mesylate, Dihydroestosterone, Dihydrostreptomycin Sulfate, Dihydrotachysterol, dihydrotaxol, 9-, Dilantin, Dilevalol Hydrochloride, Diltiazem Hydrochloride, Dimefadane, Dimefline Hydrochloride, Dimenhydrinate, Dimercaprol, Dimethadione, Dimethindene Maleate, Dimethisterone, dimethyl prostaglandin A1, Dimethyl Sulfoxide, dimethylhomospermine, dimiracetam, Dimoxamine Hydrochloride, Dinoprost, Dinoprostone, Dioxadrol Hydrochloride, dioxamycin, Diphenhydramine Citrate, Diphenidol, Diphenoxylate Hydrochloride, diphenyl spiromustine, Dipivefin Hydrochloride, Dipivefrin, dipliencyprone, diprafenone, dipropylnorspermine, Dipyridamole, Dipyrithione, Dipyrone, dirithromycin, discodermolide, Disobutamide, Disofenin, Disopyramide, Disoxaril, disulfiram, Ditekiren, Divalproex Sodium, Dizocilpine Maleate, Dobutamine, docarpamine, Docebenone, Docetaxel, Doconazole, docosanol, dofetilide, dolasetron, Ebastine, ebiratide, ebrotidine, ebselen, ecabapide, ecabet, ecadotril, ecdisteron, echicetin, echistatin, Echothiophate Iodide, Eclanamine Maleate, Eclazolast, ecomustine, Econazole, ecteinascidin 722, edaravone, Edatrexate, edelfosine, Edifolone Acetate, edobacomab, Edoxudine, edrecolomab, Edrophonium Chloride, edroxyprogesteone Acetate, efegatran, eflornithine, efonidipine, egualcen, Elantrine, eleatonin, elemene, eletriptan, elgodipine, eliprodil, Elsamitrucin, eltenae, Elucaine, emalkalim, emedastine, Emetine Hydrochloride, emiglitate, Emilium Tosylate, emitefur, emoctakin, empagliflozin, Enadoline Hydrochloride, enalapril, Enalaprilat, Enalkiren, enazadrem, Encyprate, Endralazine Mesylate, Endrysone, Enflurane, englitazone, Enilconazole, Enisoprost, Enlimomab, Enloplatin, Enofelast, Enolicam Sodium, Enoxacin, enoxacin, enoxaparin sodium, Enoxaparin Sodium, Enoximone, Enpiroline Phosphate, Enprofylline, Enpromate, entacapone, enterostatin, Enviradene, Enviroxime, Ephedrine, Epicillin, Epimestrol, Epinephrine, Epinephryl Borate, Epipropidine, Epirizole, epirubicin, Epitetracycline Hydrochloride, Epithiazide, Epoetin Alfa, Epoetin Beta, Epoprostenol, Epoprostenol Sodium, epoxymexrenone, epristeride, Eprosartan, eptastigmine, equilenin, Equilin, Erbulozole, erdosteine, Ergoloid Mesylates, Ergonovine Maleate, Ergotamine Tartrate, ersentilide, Ersofermin, erythritol, Erythrityl Tetranitrate, Erythromycin, Esmolol Hydrochloride, Esorubicin Hydrochloride, Esproquin Hydrochloride, Estazolam, Estradiol, Estramustine, estramustine analogue, Estrazinol Hydrobromide, Estriol, Estrofurate, estrogen agonists, estrogen antagonists, Estrogens, Conjugated Estrogens, Esterified Estrone, Estropipate, esuprone, Etafedrine Hydrochloride, Etanidazole, etanterol, Etarotene, Etazolate Hydrochloride, Eterobarb, ethacizin, Ethacrynate Sodium, Ethacrynic Acid, Ethambutol Hydrochloride, Ethamivan, Ethanolamine Oleate, Ethehlorvynol, Ether, Ethinyl estradiol, Ethiodized Oil, Ethionamide, Ethonam Nitrate, Ethopropazine Hydrochloride, Ethosuximide, Ethotoin, Ethoxazene Hydrochloride, Ethybenztropine, Ethyl Chloride, Ethyl Dibunate, Ethylestrenol, Ethyndiol, Ethynerone, Ethynodiol Diacetate, Etibendazole, Etidocaine, Etidronate Disodium, Etidronic Acid, Etifenin, Etintidine Hydrochloride, etizolam, Etodolac, Etofenamate, Etoformin Hydrochloride, Etomidate, Etonogestrel, Etoperidone Hydrochloride, Etoposide, Etoprine, Etoxadrol Hydrochloride, Etozolin, etrabamine, Etretinate, Etryptamine Acetate, Eucatropine Hydrochloride, Eugenol, Euprocin Hydrochloride, eveminomicin, Exametazime, examorelin, Exaprolol Hydrochloride, exemestane, fadrozole, faeriefungin, Famciclovir, Famotidine, Fampridine, fantofarone, Fantridone Hydrochloride, faropenem, fasidotril, fasudil, fazarabine, fedotozine, felbamate, Felbinac, Felodipine, Felypres sin, Fenalamide, Fenamole, Fenbendazole, Fenbufen, Fencibutirol, Fenclofenac, Fenclonine, Fenclorac, Fendosal, Fenestrel, Fenethylline Hydrochloride, Fenfluramine Hydrochloride, Fengabine, Fenimide, Fenisorex, Fenmetozole Hydrochloride, Fenmetramide, Fenobam, Fenoctimine Sulfate, fenofibrate, fenoldopam, Fenoprofen, Fenoterol, Fenpipalone, Fenprinast Hydrochloride, Fenprostalene, Fenquizone, fenretinide, fenspiride, Fentanyl Citrate, Fentiazac, Fenticlor, fenticonazole, Fenyripol Hydrochloride, fepradinol, ferpifosate sodium, ferristene, ferrixan, Ferrous Sulfate, Dried, Ferumoxides, ferumoxsil, Fetoxylate Hydrochloride, fexofenadine, Fezolamine Fumarate, Fiacitabine, Fialuridine, Fibrinogen 1 125, filgrastim, Filipin, finasteride, Flavodilol Maleate, flavopiridol, Flavoxate Hydrochloride, Flazalone, flecainide, flerobuterol, Fleroxacin, flesinoxan, Flestolol Sulfate, Fletazepam, flezelastine, flobufen, Floctafenine, flomoxef, Flordipine, florfenicol, florifenine, flosatidil, Flosequinan, Floxacillin, Floxuridine, fluasterone, Fluazacort, Flubanilate Hydrochloride, Flubendazole, Flucindole, Flucloronide, Fluconazole, Flucytosine, Fludalanine, Fludarabine Phosphate, Fludazonium Chloride, Fludeoxyglucose F 18, Fludorex, Fludrocortisone Acetate, Flufenamic Acid, Flufenisal, Flumazenil, flumecinol, Flumequine, Flumeridone, Flumethasone, Flumetramide, Flumezapine, Fluminorex, Flumizole, Flumoxonide, flunarizine, Flunidazole, Flunisolide, Flunitrazepam, Flunixin, fluocalcitriol, Fluocinolone Acetonide, Fluocinonide, Fluocortin Butyl, Fluocortolone, Fluorescein, fluorodaunorunicin hydrochloride, Fluorodopa F 18, Fluorometholone, Fluorouracil, Fluotracen Hydrochloride, Fluoxetine, Fluoxymesterone, fluparoxan, Fluperamide, Fluperolone Acetate, Fluphenazine Decanoate, flupirtine, Fluprednisolone, Fluproquazone, Fluprostenol Sodium, Fluquazone, Fluradoline Hydrochloride, Flurandrenolide, Flurazepam Hydrochloride, Flurbiprofen, Fluretofen, flurithromycin, Flurocitabine, Flurofamide, Flurogestone Acetate, Flurothyl, Fluroxene, Fluspiperone, Fluspirilene, Fluticasone Propionate, flutrimazole, Flutroline, fluvastatin, Fluvastatin Sodium, fluvoxamine, Fluzinamide, Folic Acid, Follicle regulatory protein, Folliculostatin, Fomepizole, Fonazine Mesylate, forasartan, forfenimex, forfenirmex, formestane, Formocortal, formoterol, Fosarilate, Fosazepam, Foscarnet Sodium, fosfomycin, Fosfonet Sodium, fosinopril, Fosinoprilat, fosphenyloin, Fosquidone, Fostedil, fostriecin, fotemustine, Fuchsin, Basic, Fumoxicillin, Fungimycin, Furaprofen, Furazolidone, Furazolium Chloride, Furegrelate Sodium, Furobufen, Furodazole, Furosemide, Fusidate Sodium, Fusidic Acid, gabapentin, Gadobenate Dimeglumine, gadobenic acid, gadobutrol, Gadodiamide, gadolinium texaphyrin, Gadopentetate Dimegiumine, gadoteric acid, Gadoteridol, Gadoversetamide, galantamine, galdansetron, Galdansetron Hydrochloride, Gallamine Triethiodide, gallium nitrate, gallopamil, galocitabine, Gamfexine, gamolenic acid, Ganciclovir, ganirelix, gelatinase inhibitors, Gemcadiol, Gemcitabine, Gemeprost, Gemfibrozil, Gentamicin Sulfate, Gentian Violet, gepirone, Gestaclone, Gestodene, Gestonorone Caproate, Gestrinone, Gevotroline Hydrochloride, girisopam, glaspimod, glaucocalyxin A, Glemanserin, Gliamilide, Glibornuride, Glicetanile Sodium, Gliflumide, Glimepiride, Glipizide, Gloximonam, Glucagon, glutapyrone, glutathione inhibitors, Glutethimide, Glyburide, glycopine, glycopril, Glycopyrrolate, Glyhexamide, Glymidine Sodium, Glyoctamide, Glyparamide, Gold Au 198, Gonadoctrinins, Gonadorelin, Gonadotropins, Goserelin, Gramicidin, Granisetron, grepafloxacin, Griseofulvin, Guaiapate, Guaithylline, Guanabenz, Guanabenz Acetate, Guanadrel Sulfate, Guancydine, Guanethidine Monosulfate, Guanfacine Hydrochloride, Guanisoquin Sulfate, Guanoclor Sulfate, Guanoctine Hydrochloride, Guanoxabenz, Guanoxan Sulfate, Guanoxyfen Sulfate, Gusperimus Trihydrochloride, Halazepam, Halcinonide, halichondrin B, Halobetasol Propionate, halofantrine, Halofantrine Hydrochloride, Halofenate, Halofuginone Hydrobromide, halomon, Halopemide, Haloperidol, halopredone, Haloprogesterone, Haloprogin, Halothane, Halquinols, Hamycin, Han memopausal gonadotropins, hatomamicin, hatomarubigin A, hatomarubigin B, hatomarubigin C, hatomarubigin D, Heparin Sodium, hepsulfam, heregulin, Hetacillin, Heteronium Bromide, Hexachlorophene: Hydrogen Peroxide, Hexafluorenium Bromide, hexamethylene bisacetamide, Hexedine, Hexobendine, Hexoprenaline Sulfate, Hexylresorcinol, Histamine Phosphate, Histidine, Histoplasmin, Histrelin, Homatropine Hydrobromide, Hoquizil Hydrochloride, Human chorionic gonadotropin, Hycanthone, Hydralazine Hydrochloride, Hydralazine Polistirex, Hydrochlorothiazide, Hydrocodone Bitartrate, Hydrocortisone, Hydroflumethiazide, Hydromorphone Hydrochloride, Hydroxyamphetamine Hydrobromide, Hydroxychloroquine Sulfate, Hydroxyphenamate, Hydroxyprogesterone Caproate, Hydroxyurca, Hydroxyzine Hydrochloride, Hymecromone, Hyoscyamine, hypericin, Ibafloxacin, ibandronic acid, ibogaine, Ibopamine, ibudilast, Ibufenac, Ibuprofen, Ibutilide Fumarate, Icatibant Acetate, Ichthammol, Icotidine, idarubicin, idoxifene, Idoxuridine, idramantone, lemefloxacin, lesopitron, Ifetroban, Ifosfamide, Ilepeimide, illimaquinone, ilmofosine, ilomastat, Ilonidap, iloperidone, iloprost, Imafen Hydrochloride, Imazodan Hydrochloride, imidapril, imidazenil, imidazoacridones, Imidecyl Iodine, Imidocarb Hydrochloride, Imidoline Hydrochloride, Imidurea, Imiloxan Hydrochloride, Imipenem, Imipramine Hydrochloride, imiquimod, immunostimulant peptides, Impromidine Hydrochloride, Indacrinone, Indapamide, Indecainide Hydrochloride, Indeloxazine Hydrochloride, Indigotindisulfonate Sodium, indinavir, Indocyanine Green, Indolapril Hydrochloride, Indolidan, indometacin, Indomethacin Sodium, Indoprofen, indoramin, Indorenate Hydrochloride, Indoxole, Indriline Hydrochloride, inocoterone, inogatran, inolimomab, Inositol Niacinate, Insulin, interferons, interleukins, Intrazole, Intriptyline Hydrochloride, iobenguane, Iobenzamic Acid, iobitridol, locarmate Meglumine, locarmic Acid, locetamic Acid, Iodamide, Iodine, Iodipamide Meglumine, Iodixanol, iodoamiloride, Iodoantipyrine I 131, Iodocholesterol I 131, iododoxorubicin, Iodohippurate Sodium I 131, Iodopyracet I 125, Iodoquinol, lodoxamate Meglumine, lodoxamie Acid, loglicic Acid, lofetamine Hydrochloride I 123, iofratol, loglucol, loglucomide, loglycamic Acid, logulamide, Iohexol, iomeprol, lomethin I 125, Iopamidol, lopanoic Acid, iopentol, Iophendylate, loprocemic Acid, iopromide, lopronic Acid, lopydol, lopydone, iopyrol, losefamic Acid, loseric Acid, Iosulamide Meglumine, Iosumetic Acid, Iotasul, Iotetric Acid, Iothalamate Sodium, Iothalamic Acid, iotriside, Iotrolan, Iotroxic Acid, Iotyrosine I 131, Ioversol, Ioxaglate Sodium, Ioxaglate Meglumine, loxaglic Acid, ioxilan, loxotrizoic Acid, ipazilide, ipenoxazone, ipidacrine, Ipodate Calcium, ipomeanol, 4-, Ipratropium Bromide, ipriflavone, Iprindole, Iprofenin, Ipronidazole, Iproplatin, Iproxamine Hydrochloride, ipsapirone, irbesartan, irinotecan, irloxacin, iroplact, irsogladine, Irtemazole, isalsteine, Isamoxole, isbogrel, Isepamicin, isobengazole, Isobutamben, Isocarboxazid, Isoconazole, Isoetharine, isofloxythepin, Isoflupredone Acetate, Isoflurane, Isoflurophate, isohomohalicondrin B, Isoleucine, Isomazole Hydrochloride, Isomylamine Hydrochloride, Isoniazid, Isopropamide Iodide, Isopropyl Alcohol, isopropyl unoprostone, Isoproterenol Hydrochloride, Isosorbide, Isosorbide Mononitrate, Isotiquimide, Isotretinoin, Isoxepac, Isoxicam, Isoxsuprine Hydrochloride, isradipine, itameline, itasetron, Itazigrel, itopride, Itraconazole, Ivermectin, jasplakinolide, Josamycin, kahalalide F, Kalafungin, Kanamycin Sulfate, Ketamine Hydrochloride, Ketanserin, Ketazocine, Ketazolam, Kethoxal, Ketipramine Fumarate, Ketoconazole, Ketoprofen, Ketorfanol, ketorolac, Ketotifen Fumarate, Kitasamycin, Labetalol Hydrochloride, Lacidipine, lacidipine, lactitol, lactivicin, laennec, lafutidine, lamellarin-N triacetate, lamifiban, Lamivudine, Lamotrigine, lanoconazole, Lanoxin, lanperisone, lanreotide, Lansoprazole, latanoprost, lateritin, laurocapram, Lauryl Isoquinolinium Bromide, Lavoltidine Succinate, lazabemide, Lecimibide, leinamycin, lemildipine, leminoprazole, lenercept, Leniquinsin, lenograstim, Lenperone, lentinan sulfate, leptin, leptolstatin, lercanidipine, Lergotrile, lerisetron, Letimide Hydrochloride, letrazuril, letrozole, Leucine, leucomyzin, Leuprolide Acetate, leuprolide+estrogen+progesterone, leuprorelin, Levamfetamine Succinate, levamisole, Levdobutamine Lactobionate, Leveromakalim, levetiracetam, Leveycloserine, levobetaxolol, levobunolol, levobupivacaine, levocabastine, levocarnitine, Levodopa, levodropropizine, levofloxacin, Levofuraltadone, Levoleucovorin Calcium, Levomethadyl Acetate, Levomethadyl Acetate Hydrochloride, levomoprolol, Levonantradol Hydrochloride, Levonordefrin, Levonorgestrel, Levopropoxyphene Napsylate, Levopropylcillin Potassium, levormeloxifene, Levorphanol Tartrate, levosimendan, levosulpiride, Levothyroxine Sodium, Levoxadrol Hydrochloride, Lexipafant, Lexithromycin, liarozole, Libenzapril, Lidamidine Hydrochloride, Lidocaine, Lidofenin, Lidoflazine, Lifarizine, Lifibrate, Lifibrol, Linarotene, Lincomycin, linear polyamine analogue, Linogliride, Linopirdine, linotroban, linsidomine, lintitript, lintopride, Liothyronine I 125, liothyronine sodium, Liotrix, lirexapride, lisinopril, lissoclinamide 7, Lixazinone Sulfate, lobaplatin, Lobenzarit Sodium, Lobucavir, Lodelaben, lodoxamide, Lofemizole Hydrochloride, Lofentanil Oxalate, Lofepramine Hydrochloride, Lofexidine Hydrochloride, lombricine, Lomefloxacin, lomerizine, Lometraline Hydrochloride, lometrexol, Lomofungin, Lomoxicam, Lomustine, Lonapalene, lonazolac, lonidamine, Loperamide Hydrochloride, loracarbef, Lorajmine Hydrochloride, loratadine, Lorazepam, Lorbamate, Lorcainide Hydrochloride, Loreclezole, Loreinadol, lorglumide, Lormetazepam, Lornoxicam, lornoxicam, Lortalamine, Lorzafone, losartan, losigamone, losoxantrone, Losulazine Hydrochloride, loteprednol, lovastatin, loviride, Loxapine, Loxoribine, lubeluzole, Lucanthone Hydrochloride, Lufironil, Lurosetron Mesylate, lurtotecan, luteinizing hormone, lutetium, Lutrelin Acetate, luzindole, Lyapolate Sodium, Lycetamine, lydicamycin, Lydimycin, Lynestrenol, Lypressin, Lysine, lysofylline, lysostaphin, lytic peptides, Maduramicin, Mafenide, magainin 2 amide, Magnesium Salicylate, Magnesium Sulfate, magnolol, maitansine, Malethamer, mallotochromene, mallotojaponin, Malotilate, malotilate, mangafodipir, manidipine, maniwamycin A, Mannitol, mannostatin A, manumycin E, manumycin F, mapinastine, Maprotiline, marimastat, Martek 8708, Martek 92211, Masoprocol, maspin, massetolide, matrilysin inhibitors, Maytansine, Mazapertine Succiniate, Mazindol, Mebendazole, Mebeverine Hydrochloride, Mebrofenin, Mebutamate, Mecamylamine Hydrochloride, Mechlorethamine Hydrochloride, Meclocycline, Meclofenamate Sodium, Mecloqualone, Meclorisone Dibutyrate, Medazepam Hydrochloride, Medorinone, Medrogestone, Medroxalol, Medroxyprogesterone, Medrysone, Meelizine Hydrochloride, Mefenamic Acid, Mefenidil, Mefenorex Hydrochloride, Mefexamide, Mefloquine Hydrochloride, Mefruside, Megalomicin Potassium Phosphate, Megestrol Acetate, Meglumine, Meglutol, Melengestrol Acetate, Melitracen Hydrochloride, Melphalan, Memotine Hydrochloride, Menabitan Hydrochloride, Menoctone, menogaril, Menotropins, Meobentine Sulfate, Mepartricin, Mepenzolate Bromide, Meperidine Hydrochloride, Mephentermine Sulfate, Mephenyloin, Mephobarbital, Mepivacaine Hydrochloride, Meprobamate, Meptazinol Hydrochloride, Mequidox, Meralein Sodium, merbarone, Mercaptopurine, Mercufenol Chloride, Mercury, Ammoniated, Merisoprol Hg 197, Meropenem, Mesalamine, Meseclazone, Mesoridazine, Mesterolone, Mestranol, Mesuprine Hydrochloride, Metalol Hydrochloride, Metaproterenol Polistirex, Metaraminol Bitartrate, Metaxalone, Meteneprost, meterelin, Metformin, Methacholine Chloride, Methacycline, Methadone Hydrochloride, Methadyl Acetate, Methalthiazide, Methamphetamine Hydrochloride, Methaqualone, Methazolamide, Methdilazine, Methenamine, Methenolone Acetate, Methetoin, Methicillin Sodium, Methimazole, methioninase, Methionine, Methisazone, Methixene Hydrochloride, Methocarbamol, Methohexital Sodium, Methopholine, Methotrexate, Methotrimeprazine, methoxatone, Methoxyflurane, Methsuximide, Methyclothiazide, Methyl Palmoxirate, Methylatropine Nitrate, Methylbenzethonium Chloride, Methyldopa, Methyldopate Hydrochloride, Methylene Blue, Methylergonovine Maleate, methylhistamine, R-alpha, methylinosine monophosphate, Methylphenidate Hydrochloride, Methylprednisolone, Methyltestosterone, Methynodiol Diacelate, Methysergide, Methysergide Maleate, Metiamide, Metiapine, Metioprim, metipamide, Metipranolol, Metizoline Hydrochloride, Metkephamid Acetate, metoclopramide, Metocurine Iodide, Metogest, Metolazone, Metopimazine, Metoprine, Metoprolol, Metoquizine, metrifonate, Metrizamide, Metrizoate Sodium, Metronidazole, Meturedepa, Metyrapone, Metyrosine, Mexiletine Hydrochloride, Mexrenoate Potassium, Mezlocillin, mfonelic Acid, Mianserin Hydrochloride, mibefradil, Mibefradil Dihydrochloride, Mibolerone, michellamine B, Miconazole, microcolin A, Midaflur, Midazolam Hydrochloride, midodrine, mifepristone, Mifobate, miglitol, milacemide, milameline, mildronate, Milenperone, Milipertine, milnacipran, Milrinone, miltefosine, Mimbane Hydrochloride, minaprine, Minaxolone, Minocromil, Minocycline, Minoxidil, Mioflazine Hydrochloride, miokamycin, mipragoside, mirfentanil, mirimostim, Mirincamycin Hydrochloride, Mirisetron Maleate, Mirtazapine, mismatched double stranded RNA, Misonidazole, Misoprostol, Mitindomide, Mitocarcin, Mitocromin, Mitogillin, mitoguazone, mitolactol, Mitomalcin, Mitomycin, mitonafide, Mitosper, Mitotane, mitoxantrone, mivacurium chloride, mivazerol, mixanpril, Mixidine, mizolastine, mizoribine, Moclobemide, modafinil, Modaline Sulfate, Modecainide, moexipril, mofarotene, Mofegiline Hydrochloride, mofezolac, molgramostim, Molinazone, Molindone Hydrochloride, Molsidomine, mometasone, Monatepil Maleate, Monensin, Monoctanoin, Montelukast Sodium, montirelin, mopidamol, moracizine, Morantel Tartrate, Moricizine, Morniflumate, Morphine Sulfate, Morrhuate Sodium, mosapramine, mosapride, motilide, Motretinide, Moxalactam Disodium, Moxazocine, moxiraprine, Moxnidazole, moxonidine, Mumps Skin Test Antigen, mustard anticancer agent, Muzolimine, mycaperoxide B, Mycophenolic Acid, myriaporone, Nabazenil, Nabilone, Nabitan Hydrochloride, Naboctate Hydrochloride, Nabumetone, N-acetyldinaline, Nadide, nadifloxacin, Nadolol, nadroparin calcium, nafadotride, nafamostat, nafarelin, Nafcillin Sodium, Nafenopin, Naf imidone Hydrochloride, Naflocort, Nafomine Malate, Nafoxidine Hydrochloride, Nafronyl Oxalate, Naftifine Hydrochloride, naftopidil, naglivan, nagrestip, Nalbuphine Hydrochloride, Nalidixate Sodium, Nalidixic Acid, nalmefene, Nalmexone Hydrochloride, naloxone+pentazocine, Naltrexone, Namoxyrate, Nandrolone Phenpropionate, Nantradol Hydrochloride, Napactadine Hydrochloride, napadisilate, Napamezole Hydrochloride, napaviin, Naphazoline Hydrochloride, naphterpin, Naproxen, Naproxol, napsagatran, Naranol Hydrochloride, Narasin, naratriptan, nartograstim, nasaruplase, Natamycin, nateplase, Naxagolide Hydrochloride, Nebivolol, Nebramycin, nedaplatin, Nedocromil, Nefazodone Hydrochloride, Neflumozide Hydrochloride, Nefopam Hydrochloride, Nelezaprine Maleate, Nemazoline Hydrochloride, nemorubicin, Neomycin Palmitate, Neostigmine Bromide, neridronic acid, Netilmicin Sulfate, neutral endopeptidase, Neutramycin, Nevirapine, Nexeridine Hydrochloride, Niacin, Nibroxane, Nicardipine Hydrochloride, Nicergoline, Niclosamide, Nicorandil, Nicotinyl Alcohol, Nifedipine, Nifirmerone, Nifluridide, Nifuradene, Nifuraldezone, Nifuratel, Nifuratrone, Nifurdazil, Nifurimide, Nifurpirinol, Nifurquinazol, Nifurthiazole, nilutamide, Nilvadipine, Nimazone, Nimodipine, niperotidine, niravoline, Niridazole, nisamycin, Nisbuterol Mesylate, nisin, Nisobamate, Nisoldipine, Nisoxetine, Nisterime Acetate, Nitarsone, nitazoxamide, nitecapone, Nitrafudam Hydrochloride, Nitralamine Hydrochloride, Nitramisole Hydrochloride, Nitrazepam, Nitrendipine, Nitrocycline, Nitrodan, Nitrofurantoin, Nitrofurazone, Nitroglycerin, Nitromersol, Nitromide, Nitromifene Citrate, Nitrous Oxide, nitroxide antioxidant, nitrullyn, Nivazol, Nivimedone Sodium, Nizatidine, Noberastine, Nocodazole, Nogalamycin, Nolinium Bromide, Nomifensine Maleate, Noracymethadol Hydrochloride, Norbolethone, Norepinephrine Bitartrate, Norethindrone, Norethynodrel, Norfloxacin, Norflurane, Norgestimate, Norgestomet, Norgestrel, Nortriptyline Hydrochloride, Noscapine, Novobiocin Sodium, N-substituted benzaimides, Nufenoxole, Nylestriol, Nystatin, O6-benzylguanine, Obidoxime Chloride, Ocaperidone, Ocfentanil Hydrochloride, Ocinaplon, Octanoic Acid, Octazamide, Octenidine Hydrochloride, Octodrine, Octreotide, Octriptyline Phosphate, Ofloxacin, Oformine, okicenone, Olanzapine, oligonucleotides, olopatadine, olprinone, olsalazine, Olsalazine Sodium, Olvanil, omeprazole, onapristone, ondansetron, Ontazolast, Oocyte maturation inhibitor, Opipramol Hydrochloride, oracin, Orconazole Nitrate, Orgotein, Orlislat, Ormaplatin, Ormetoprim, Ornidazole, Orpanoxin, Orphenadrine Citrate, osaterone, otenzepad, Oxacillin Sodium, Oxagrelate, oxaliplatin, Oxamarin Hydrochloride, oxamisole, Oxamniquine, oxandrolone, Oxantel Pamoate, Oxaprotiline Hydrochloride, Oxaprozin, Oxarbazole, Oxatomide, oxaunomycin, Oxazepam, oxcarbazepine, Oxendolone, Oxethazaine, Oxetorone Fumarate, Oxfendazole, Oxfenicine, Oxibendazole, oxiconazole, Oxidopamine, Oxidronic Acid, Oxifungin Hydrochloride, Oxilorphan, Oximonam, Oximonam Sodium, Oxiperomide, oxiracetam, Oxiramide, Oxisuran, Oxmetidine Hydrochloride, oxodipine, Oxogestone Phenpropionate, Oxolinic Acid, Oxprenolol Hydrochloride, Oxtriphylline, Oxybutynin Chloride, Oxychlorosene, Oxycodone, Oxymetazoline Hydrochloride, Oxymetholone, Oxymorphone Hydrochloride, Oxypertine, Oxyphenbutazone, Oxypurinol, Oxytetracycline, Oxytocin, ozagrel, Ozolinone, Paclitaxel, palauamine, Paldimycin, palinavir, palmitoylrhizoxin, Palmoxirate Sodium, pamaqueside, Pamatolol Sulfate, pamicogrel, Pamidronate Disodium, pamidronic acid, Panadiplon, panamesine, panaxytriol, Pancopride, Pancuronium Bromide, panipenem, pannorin, panomifene, pantethine, pantoprazole, Papaverine Hydrochloride, parabactin, Parachlorophenol, Paraldehyde, Paramethasone Acetate, Paranyline Hydrochloride, Parapenzolate Bromide, Pararosaniline Pamoate, Parbendazole, Parconazole Hydrochloride, Paregoric, Pareptide Sulfate, Pargyline Hydrochloride, parnaparin sodium, Paromomycin Sulfate, Paroxetine, parthenolide, Partricin, Paulomycin, pazelliptine, Pazinaclone, Pazoxide, pazufloxacin, pefloxacin, pegaspargase, Pegorgotein, Pelanserin Hydrochloride, peldesine, Peliomycin, Pelretin, Pelrinone Hydrochloride, Pemedolac, Pemerid Nitrate, pemirolast, Pemoline, Penamecillin, Penbutolol Sulfate, Penciclovir, Penfluridol, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentabamate, Pentaerythritol Tetranitrate, pentafuside, pentamidine, pentamorphone, Pentamustine, Pentapiperium Methylsulfate, Pentazocine, Pentetic Acid, Pentiapine Maleate, pentigetide, Pentisomicin, Pentizidone Sodium, Pentobarbital, Pentomone, Pentopril, pentosan, pentostatin, Pentoxifylline, Pentrinitrol, pentrozole, Peplomycin Sulfate, Pepstatin, perflubron, perfofamide, Perfosfamide, pergolide, Perhexiline Maleate, perillyl alcohol, Perindopril, perindoprilat, Perlapine, Permethrin, perospirone, Perphenazine, Phenacemide, phenaridine, phenazinomycin, Phenazopyridine Hydrochloride, Phenbutazone Sodium Glycerate, Phencarbamide, Phencyclidine Hydrochloride, Phendimetrazine Tartrate, Phenelzine Sulfate, Phenmetrazine Hydrochloride, Phenobarbital, Phenoxybenzamine Hydrochloride, Phenprocoumon, phenserine, phensuccinal, Phensuximide, Phentermine, Phentermine Hydrochloride, phentolamine mesilate, Phentoxifylline, Phenyl Aminosalicylate, phenylacetate, Phenylalanine, phenylalanyl ketoconazole, Phenylbutazone, Phenylephrine Hydrochloride, Phenylpropanolamine Hydrochloride, Phenylpropanolamine Polistirex, Phenyramidol Hydrochloride, Phenyloin, phosphatase inhibitors, Physostigmine, picenadol, picibanil, Picotrin Diolamine, picroliv, picumeterol, pidotimod, Pifamine, Pilocarpine, pilsicainide, pimagedine, Pimetine Hydrochloride, pimilprost, Pimobendan, Pimozide, Pinacidil, Pinadoline, Pindolol, pinnenol, pinocebrin, Pinoxepin Hydrochloride, pioglitazone, Pipamperone, Pipazethate, pipecuronium bromide, Piperacetazine, Piperacillin Sodium, Piperamide Maleate, piperazine, Pipobroman, Piposulfan, Pipotiazine Palmitate, Pipoxolan Hydrochloride, Piprozolin, Piquindone Hydrochloride, Piquizil Hydrochloride, Piracetam, Pirandamine Hydrochloride, pirarubicin, Pirazmonam Sodium, Pirazolac, Pirbenicillin Sodium, Pirbuterol Acetate, Pirenperone, Pirenzepine Hydrochloride, piretamide, Pirfenidone, Piridicillin Sodium, Piridronate Sodium, Piriprost, piritrexim, Pirlimycin Hydrochloride, pirlindole, pirmagrel, Pirmenol Hydrochloride, Pirnabine, Piroctone, Pirodavir, pirodomast, Pirogliride Tartrate, Pirolate, Pirolazamide, Piroxantrone Hydrochloride, Piroxicam, Piroximone, Pirprofen, Pirquinozol, Pirsidomine, Prenylamine, Pituitary, Posterior, Pivampicillin Hydrochloride, Pivopril, Pizotyline, placetin A, platinum compounds, platinum-triamine complex, Plicamycin, Plomestane, Pobilukast Edamine, Podofilox, Poisonoak Extract, Poldine Methylsulfate, Poliglusam, Polignate Sodium, Polymyxin B Sulfate, Polythiazide, Ponalrestat, Porfimer Sodium, Porfiromycin, Potassium Chloride, Potassium Iodide, Potassium Permanganate, Povidone-Iodine, Practolol, Pralidoxime Chloride, Pramiracetam Hydrochloride, Pramoxine Hydrochloride, Pranolium Chloride, Pravadoline Maleate, Pravastatin (Pravachol), Prazepam, Prazosin, Prazosin Hydrochloride, Prednazate, Prednicarbate, Prednimustine, Prednisolone, Prednisone, Prednival, Pregnenolone Succiniate, Prenalterol Hydrochloride, Pridefine Hydrochloride, Prifelone, Prilocalne Hydrochloride, Prilosec, Primaquine Phosphate, Primidolol, Primidone, Prinivil, Prinomide Tromethamine, Prinoxodan, Prizidilol Hydrochloride, Proadifen Hydrochloride, Probenecid, Probicromil Calcium, Probucol, Procainamide Hydrochloride, Procaine Hydrochloride, Procarbazine Hydrochloride, Procaterol Hydrochloride, Prochlorperazine, Procinonide, Proclonol, Procyclidine Hydrochloride, Prodilidine Hydrochloride, Prodolic Acid, Profadol Hydrochloride, Progabide, Progesterone, Proglumide, Proinsulin Human, Proline, Prolintane Hydrochloride, Promazine Hydrochloride, Promethazine Hydrochloride, Propafenone Hydrochloride, propagermanium, Propanidid, Propantheline Bromide, Proparacaine Hydrochloride, Propatyl Nitrate, propentofylline, Propenzolate Hydrochloride, Propikacin, Propiomazine, Propionic Acid, propionylcarnitine, L-, propiram, propiram+paracetamol, propiverine, Propofol, Propoxycaine Hydrochloride, Propoxyphene Hydrochloride, Propranolol Hydrochloride, Propulsid, propyl bis-acridone, Propylhexedrine, Propyliodone, Propylthiouracil, Proquazone, Prorenoate Potassium, Proroxan Hydrochloride, Proscillaridin, Prostalene, prostratin, Protamine Sulfate, protegrin, Protirelin, protosufloxacin, Protriptyline Hydrochloride, Proxazole, Proxazole Citrate, Proxicromil, Proxorphan Tartrate, prulifloxacin, Pseudoephedrine Hydrochloride, Puromycin, purpurins, Pyrabrom, Pyrantel Pamoate, Pyrazinamide, Pyrazofurin, pyrazoloacridine, Pyridostigmine Bromide, Pyrilamine Maleate, Pyrimethamine, Pyrinoline, Pyrithione Sodium, Pyrithione Zinc, Pyrovalerone Hydrochloride, Pyroxamine Maleate, Pyrrocaine, Pyrroliphene Hydrochloride, Pyrrolnitrin, Pyrvinium Pamoate, Quadazocine Mesylate, Quazepam, Quazinone, Quazodine, Quazolast, quetiapine, quiflapon, quinagolide, Quinaldine Blue, quinapril, Quinaprilat, Quinazosin Hydrochloride, Quinbolone, Quinctolate, Quindecamine Acetate, Quindonium Bromide, Quinelorane Hydrochloride, Quinestrol, Quinfamide, Quingestanol Acetate, Quingestrone, Quinidine Gluconate, Quinielorane Hydrochloride, Quinine Sulfate, Quinpirole Hydrochloride, Quinterenol Sulfate, Quinuclium Bromide, Quinupristin, Quipazine Maleate, Rabeprazole Sodium, Racephenicol, Racepinephrine, raf antagonists, Rafoxamide, Ralitoline, raloxifene, raltitrexed, ramatroban, Ramipril, Ramoplanin, ramosetron, ranelic acid, Ranimycin, Ranitidine, ranolazine, Rauwolfia Serpentina, recainam, Recainam Hydrochloride, Reclazepam, regavirumab, Regramostim, Relaxin, Relomycin, Remacemide Hydrochloride, Remifentanil Hydrochloride, Remiprostol, Remoxipride, Repirinast, Repromicin, Reproterol Hydrochloride, Reserpine, resinferatoxin, Resorcinol, retelliptine demethylated, reticulon, reviparin sodium, revizinone, rhenium Re 186 etidronate, rhizoxin, Ribaminol, Ribavirin, Riboprine, ribozymes, ricasetron, Ridogrel, Rifabutin, Rifametane, Rifamexil, Rifamide, Rifampin, Rifapentine, Rifaximin, RH retinamide, rilopirox, Riluzole, rimantadine, Rimcazole Hydrochloride, Rimexolone, Rimiterol Hydrobromide, rimoprogin, riodipine, Rioprostil, Ripazepam, ripisartan, Risedronate Sodium, risedronic acid, Risocaine, Risotilide Hydrochloride, rispenzepine, Risperdal, Risperidone, Ritanserin, ritipenem, Ritodrine, Ritolukast, ritonavir, rizatriptan benzoate, Rocastine Hydrochloride, Rocuronium Bromide, Rodocaine, Roflurane, Rogletimide, rohitukine, rokitamycin, Roletamicide, Rolgamidine, Rolicyprine, Rolipram, Rolitetracycline, Rolodine, Romazarit, romurtide, Ronidazole, ropinirole, Ropitoin Hydrochloride, ropivacaine, Ropizine, roquinimex, Rosaramicin, rosiglitazone, Rosoxacin, Rotoxamine, roxaitidine, Roxarsone, roxindole, roxithromycin, rubiginone B1, ruboxyl, rufloxacin, rupatidine, Rutamycin, ruzadolane, Sabeluzole, safingol, safironil, saintopin, salbutamol, R-Salcolex, Salethamide Maleate, Salicyl Alcohol, Salicylamide, Salicylate Meglumine, Salicylic Acid, Salmeterol, Salnacediin, Salsalate, sameridine, sampatrilat, Sancycline, sanfetrinem, Sanguinarium Chloride, Saperconazole, saprisartan, sapropterin, saquinavir, Sarafloxacin Hydrochloride, Saralasin Acetate, SarCNU, sarcophytol A, sargramostim, Sarmoxicillin, Sarpicillin, sarpogrelate, saruplase, saterinone, satigrel, satumomab pendetide, Schick Test Control, Scopafungin, Scopolamine Hydrobromide, Scrazaipine Hydrochloride, Sdi 1 mimetics, Secalciferol, Secobarbital, Seelzone, Seglitide Acetate, selegiline, Selegiline Hydrochloride, Selenium Sulfide, Selenomethionine Se 75, Selfotel, sematilide, semduramicin, semotiadil, semustine, sense oligonucleotides, Sepazonium Chloride, Seperidol Hydrochloride, Seprilose, Seproxetine Hydrochloride, Seractide Acetate, Sergolexole Maleate, Serine, Sermetacin, Sermorelin Acetate, sertaconazole, sertindole, sertraline, setiptiline, Setoperone, sevirumab, sevoflurane, sezolamide, Sibopirdine, Sibutramine Hydrochloride, signal transduction inhibitors, Silandrone, silipide, silteplase, Silver Nitrate, simendan, Simtrazene, Simvastatin, Sincalide, Sinefungin, sinitrodil, sinnabidol, sipatrigine, sirolimus, Sisomicin, Sitogluside, sizofiran, sobuzoxane, Sodium Amylosulfate, Sodium Iodide I 123, Sodium Nitroprusside, Sodium Oxybate, sodium phenylacetate, Sodium Salicylate, solverol, Solypertine Tartrate, Somalapor, Somantadine Hydrochloride, somatomedin B, somatomedin C, somatrem, somatropin, Somenopor, Somidobove, sonermin, Sorbinil, Sorivudine, sotalol, Soterenol Hydrochloride, Sparfloxacin, Sparfosate Sodium, sparfosic acid, Sparsomycin, Sparteine Sulfate, Spectinomycin Hydrochloride, spicamycin D, Spiperone, Spiradoline Mesylate, Spiramycin, Spirapril Hydrochloride, Spiraprilat, Spirogermanium Hydrochloride, Spiromustine, Spironolactone, Spiroplatin, Spiroxasone, splenopentin, spongistatin 1, Sprodiamide, squalamine, Stallimycin Hydrochloride, Stannous Pyrophosphate, Stannous Sulfur Colloid, Stanozolol, Statolon, staurosporine, stavudine, Steffimycin, Stenbolone Acetate, stepronin, Stilbazium Iodide, Stilonium Iodide, stipiamide, Stiripentol, stobadine, Streptomycin Sulfate, Streptonicozid, Streptonigrin, Streptozocin, stromelysin inhibitors, Strontium Chloride Sr 89, succibun, Succimer, Succinylcholine Chloride, Sucralfate, Sucrosofate Potassium, Sudoxicam, Sufentanil, Sufotidine, Sulazepam, Sulbactam Pivoxil, Sulconazole Nitrate, Sulfabenz, Sulfabenzamide, Sulfacetamide, Sulfacytine, Sulfadiazine, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, sulfasalazine, Sulfasomizole, Sulfazamet, Sulfinalol Hydrochloride, sulfinosine, Sulfinpyrazone, Sulfisoxazole, Sulfomyxin, Sulfonterol Hydrochloride, sulfoxamine, Sulinldac, Sulmarin, Sulnidazole, Suloctidil, Sulofenur, sulopenem, Suloxifen Oxalate, Sulpiride, Sulprostone, sultamicillin, Sulthiame, sultopride, sulukast, Sumarotene, sumatriptan, Suncillin Sodium, Suproclone, Suprofen, suradista, suramin, Surfomer, Suricainide Maleate, Suritozole, Suronacrine Maleate, Suxemerid Sulfate, swainsonine, symakalim, Symclosene, Symetine Hydrochloride, synthetic glycosaminoglycans, Taciamine Hydrochloride, Tacrine Hydrochloride, Tacrolimus, Talampicillin Hydrochloride, Taleranol, Talisomycin, tallimustine, Talmetacin, Talniflumate, Talopram Hydrochloride, Talosalate, Tametraline Hydrochloride, Tamoxifen, Tampramine Fumarate, Tamsulosin Hydrochloride, Tandamine Hydrochloride, tandospirone, tapgen, taprostene, Tasosartan, tauromustine, Taxane, Taxoid, Tazadolene Succinate, tazanolast, tazarotene, Tazifylline Hydrochloride, Tazobactam, Tazofelone, Tazolol Hydrochloride, Tebufelone, Tebuquine, Technetium Tc 99 m Bicisate, Teclozan, Tecogalan Sodium, Teecleukin, Teflurane, Tegafur, Tegretol, Teicoplanin, telenzepine, tellurapyrylium, telmesteine, telmisartan, telomerase inhibitors, Teloxantrone Hydrochloride, Teludipine Hydrochloride, Temafloxacin Hydrochloride, Tematropium Methyl sulfate, Temazepam, Temelastine, temocapril, Temocillin, temoporfin, temozolomide, Tenidap, Teniposide, tenosal, tenoxicam, tepirindole, Tepoxalin, Teprotide, terazosin, Terbinafine, Terbutaline Sulfate, Terconazole, terfenadine, terflavoxate, terguride, Teriparatide Acetate, terlakiren, terlipres sin, terodiline, Teroxalene Hydrochloride, Teroxirone, tertatolol, Tesicam, Tesimide, Testolactone, Testosterone, Tetracaine, tetrachlorodecaoxide, Tetracycline, Tetrahydrozoline Hydrochloride, Tetramisole Hydrochloride, Tetrazolast Meglumine, tetrazomine, Tetrofosmin, Tetroquinone, Tetroxoprim, Tetrydamine, thaliblastine, Thalidomide, Theofibrate, Theophylline, Thiabendazole, Thiamiprine, Thiamphenicol, Thiamylal, Thiazesim Hydrochloride, Thiazinamium Chloride, Thiethylperazine, Thimerfonate Sodium, Thimerosal, thiocoraline, thiofedrine, Thioguanine, thiomarinol, Thiopental Sodium, thioperamide, Thioridazine, Thiotepa, Thiothixene, Thiphenamil Hydrochloride, Thiphencillin Potassium, Thiram, Thozalinone, Threonine, Thrombin, thrombopoietin, thrombopoietin mimetic, thymalfasin, thymopoietin receptor agonist, thymotrinan, Thyromedan Hydrochloride, Thyroxine 1 125, Thyroxine 1 131, Tiacrilast, Tiacrilast Sodium, tiagabine, Tiamenidine, tianeptine, tiapafant, Tiapamil Hydrochloride, Tiaramide Hydrochloride, Tiazofurin, Tibenelast Sodium, Tibolone, Tibric Acid, Ticabesone Propionate, Ticarbodine, Ticarcillin Cresyl Sodium, Ticlatone, ticlopidine, Ticrynafen, tienoxolol, Tifurac Sodium, Tigemonam Dicholine, Tigestol, Tiletamine Hydrochloride, Tilidine Hydrochloride, tilisolol, tilnoprofen arbamel, Tilorone Hydrochloride, Tiludronate Disodium, tiludronic acid, Timefurone, Timobesone Acetate, Timolol, tin ethyl etiopurpurin, Tinabinol, Timidazole, Tinzaparin Sodium, Tioconazole, Tiodazosin, Tiodonium Chloride, Tioperidone Hydrochloride, Tiopinac, Tiospirone Hydrochloride, Tiotidine, tiotropium bromide, Tioxidazole, Tipentosin Hydrochloride, Tipredane, Tiprenolol Hydrochloride, Tiprinast Meglumine, Tipropidil Hydrochloride, Tiqueside, Tiquinamide Hydrochloride, tirandalydigin, Tirapazamine, tirilazad, tirofiban, tiropramide, titanocene dichloride, Tixanox, Tixocortol Pivalate, Tizanidine Hydrochloride, Tobramycin, Tocainide, Tocamphyl, Tofenacin Hydrochloride, Tolamolol, Tolazamide, Tolazoline Hydrochloride, Tolbutamide, Tolcapone, Tolciclate, Tolfamide, Tolgabide, lamotrigine, Tolimidone, Tolindate, Tolmetin, Tolnaftate, Tolpovidone 1 131, Tolpyrramide, Tolrestat, Tomelukast, Tomoxetine Hydrochloride, Tonazocine Mesylate, Topiramate, topotecan, Topotecan Hydrochloride, topsentin, Topterone, Toquizine, torasemide, toremifene, Torsemide, Tosifen, Tosufloxacin, totipotent stem cell factor, Tracazolate, trafermin, Tralonide, Tramadol Hydrochloride, Tramazoline Hydrochloride, trandolapril, Tranexamic Acid, Tranilast, Transcainide, translation inhibitors, traxanox, Trazodone Hydrochloride, Trazodone-HCL, Trebenzomine Hydrochloride, Trefentanil Hydrochloride, Treloxinate, Trepipam Maleate, Trestolone Acetate, tretinoin, Triacetin, triacetyluridine, Triafungin, Triamcinolone, Triampyzine Sulfate, Triamterene, Triazolam, Tribenoside, tricaprilin, Tricetamide, Trichlormethiazide, trichohyalin, triciribine, Tricitrates, Triclofenol piperazine, Triclofos Sodium, Triclonide, trientine, Trifenagrel, triflavin, Triflocin, Triflubazam, Triflumidate, Trifluoperazine Hydrochloride, Trifluperidol, Triflupromazine, Triflupromazine Hydrochloride, Trifluridine, Trihexyphenidyl Hydrochloride, Trilostane, Trimazosin Hydrochloride, trimegestone, Trimeprazine Tartrate, Trimethadione, Trimethaphan Camsylate, Trimethobenzamide Hydrochloride, Trimethoprim, Trimetozine, Trimetrexate, Trimipramine, Trimoprostil, Trimoxamine Hydrochloride, Triolein 1 125, Triolein 1 131, Trioxifene Mesylate, Tripamide, Tripelennamine Hydrochloride, Triprolidine Hydrochloride, Triptorelin, Trisulfapyrimidines, Troclosene Potassium, troglitazone, Trolamine, Troleandomycin, trombodipine, trometamol, Tropanserin Hydrochloride, Tropicamide, tropine ester, tropisetron, trospectomycin, trovafloxacin, trovirdine, Tryptophan, Tuberculin, Tubocurarine Chloride, Tubulozole Hydrochloride, tucarcsol, tulobuterol, turosteride, Tybamate, tylogenin, Tyropanoate Sodium, Tyrosine, Tyrothricin, tyrphostins, ubenimex, Uldazepam, Undecylenic Acid, Uracil Mustard, urapidil, Urea, Uredepa, uridine triphosphate, Urofollitropin, Urokinase, Ursodiol, valaciclovir, Valine, Valnoctamide, Valproate Sodium, Valproic Acid, valsartan, vamicamide, vanadeine, Vancomycin, vaninolol, Vapiprost Hydrochloride, Vapreotide, variolin B, Vasopressin, Vecuronium Bromide, velaresol, Velnacrine Maleate, venlafaxine, Veradoline Hydrochloride, veramine, Verapamil Hydrochloride, verdins, Verilopam Hydrochloride, Verlukast, Verofylline, veroxan, verteporfin, Vesnarinone, vexibinol, Vidarabine, vigabatrin, Viloxazine Hydrochloride, Vinblastine Sulfate, vinburnine citrate, Vincofos, vinconate, Vincristine Sulfate, Vindesine, Vindesine Sulfate, Vinepidine Sulfate, Vinglycinate Sulfate, Vinleurosine Sulfate, vinorelbine, vinpocetine, vintoperol, vinxaltine, Vinzolidine Sulfate, Viprostol, Virginiamycin, Viridofulvin, Viroxime, vitaxin, Volazocine, voriconazole, vorozole, voxergolide, Warfarin Sodium, Xamoterol, Xanomeline, Xanoxate Sodium, Xanthinol Niacinate, xemilofiban, Xenalipin, Xenbucin, Xilobam, ximoprofen, Xipamide, Xorphanol Mesylate, Xylamidine Tosylate, Xylazine Hydrochloride, Xylometazoline Hydrochloride, Xylose, yangambin, zabicipril, zacopride, zafirlukast, Zalcitabine, zaleplon, zalospirone, Zaltidine Hydrochloride, zaltoprofen, zanamivir, zankiren, zanoterone, Zantac, Zarirlukast, zatebradine, zatosetron, Zatosetron Maleate, zenarestat, Zenazocine Mesylate, Zeniplatin, Zeranol, Zidometacin, Zidovudine, zifrosilone, Zilantel, zilascorb, zileuton, Zimeldine Hydrochloride, Zinc Undecylenate, Zindotrine, Zinoconazole Hydrochloride, Zinostatin, Zinterol Hydrochloride, Zinviroxime, ziprasidone, Zobolt, Zofenopril Calcium, Zofenoprilat, Zolamine Hydrochloride, Zolazepam Hydrochloride, zoledronie acid, Zolertine Hydrochloride, zolmitriptan, zolpidem, Zomepirac Sodium, Zometapine, Zoniclezole Hydrochloride, Zonisamide, zopiclone, Zopolrestat, Zorbamyciin, Zorubicin Hydrochloride, zotepine, Zucapsaicin.

Further examples of antidiabetic actives include but are not limited to JTT-501 (PNU-182716) (Reglitazar), AR-H039242, MCC-555 (Netoglitazone), AR-H049020 Tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (Ragaglitazar), L-686398, L-168049, L-805645, L-054852, Demethyl asteriquinone B1 (L-783281), L-363586, KRP-297, P32/98, CRE-16336 and EML-16257.

In an embodiment, the pharmaceutical active is rizatriptan, optionally in combination with an NSAID. In an embodiment, the pharmaceutical active is testosterone. In another embodiment, the pharmaceutical active is diazepam. In another embodiment, the pharmaceutical active is pregabalin (Lyrica). In yet another embodiment, the pharmaceutical active is epinephrine. In a further embodiment, the pharmaceutical active is sildenafil. In a further embodiment, the pharmaceutical active is clobazam. In a still further embodiment, the pharmaceutical active is riluzole. In a further embodiment, the pharmaceutical active is apomorphine. In yet another embodiment, the pharmaceutical active is buprenorphine, optionally in combination with naloxone.

In one example, a self-supporting individual film dosage form including epinephrine, or its salts, prodrugs, derivatives, analogues, or esters, has a substantially equivalent pharmacokinetic profile to that of epinephrine administered by injection, for example, using an EpiPen. Epinephrine can be present in the individual film dosage form in an amount of from about 0.01 mg to about 100 mg per dosage, for example, at a 0.075 mg, 0.125 mg, 0.2 mg, 0.5 mg, 0.75 mg, 2 mg, 3.5 mg, 4 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, less than 5 mg, less than 0.5 mg, less than 0.3 mg or any combination thereof. About 30% to about 75%, about 50% to about 70%, about 50%, about 60%, about 70%, or about 75% less epinephrine may be required in the self-supporting film dosage unit, than in the corresponding autoinjection solution to achieve substantially equivalent Cmax, AUC, and Tmax values, i.e., within the range of about 80% to about 125%, at a confidence level of at least about 80%.

In another example, a self-supporting individual film dosage form containing diazepam has a substantially equivalent pharmacokinetic profile to that of a diazepam tablet or gel. Diazepam or its salts can be present in the individual film dosage form in an amount of from about 0.5 mg to about 100 mg per dosage, for example, at a 0.5 mg, 0.75 mg, 1 mg, 1.5 mg, 2 mg, 2.5 mg, 3 mg, 3.5 mg, 4 mg, 4.5 mg, 5 mg, 7.5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. About 20% to about 60%, about 30% to about 50%, about 30%, about 40%, about 50%, about 60%, or about 70% less diazepam may be required in the self-supporting film dosage unit than in the corresponding oral tablet to achieve substantially equivalent Cmax, AUC, and Tmax values, i.e., within the range of about 80% to about 125%, at a confidence level of at least about 80%.

In another example, a self-supporting individual film dosage form containing sildenafil, has a substantially equivalent pharmacokinetic profile to that of an oral tablet containing sildenafil. Sildenafil or its salts can be present in the individual film dosage form in an amount of from about 0.5 mg to about 100 mg per dosage, for example, at a 0.5 mg, 0.75 mg, 1 mg, 1.5 mg, 2 mg, 2.5 mg, 3 mg, 3.5 mg, 4 mg, 4.5 mg, 5 mg, 7.5 mg, 10 mg, 20 mg, 22 mg, 25 mg, 30 mg, 35 mg, 40 mg, 45 mg, 50 mg, 55 mg, 60 mg, 65 mg, 70 mg, 80 mg, 90 mg, 95 mg, or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. About 1% to about 30%, about 5% to about 20%, about 1%, about 5%, about 10%, about 15%, or about 20% less sildenafil may be required in the self-supporting film dosage unit than in the corresponding oral tablet to achieve substantially equivalent Cmax, AUC, and Tmax values, i.e., within the range of about 80% to about 125%, at a confidence level of at least about 80%.

In another example, a self-supporting individual film dosage form containing rizatriptan, has a substantially equivalent pharmacokinetic profile to that of an oral tablet containing rizatriptan. Rizatriptan or its salts can be present in the individual film dosage form in an amount of from about 0.5 mg to about 100 mg per dosage, for example, at a 0.5 mg, 0.75 mg, 1 mg, 1.5 mg, 2 mg, 2.5 mg, 3 mg, 3.5 mg, 4 mg, 4.5 mg, 5 mg, 7.5 mg, 10 mg, 20 mg, 22 mg, 25 mg, 30 mg, 35 mg, 40 mg, 45 mg, 50 mg, 55 mg, 60 mg, 65 mg, 70 mg, 80 mg, 90 mg, 95 mg, or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. About 40% to about 75%, about 50% to about 70%, about 40%, about 50%, about 60%, about 70%, or about 75% less rizatriptan may be required in the self-supporting film dosage unit than in the corresponding oral tablet to achieve substantially equivalent Cmax, AUC, and Tmax values, i.e., within the range of about 80% to about 125%, at a confidence level of at least about 80%.

In another example, a self-supporting individual film dosage form containing buprenorphine has a substantially equivalent pharmacokinetic profile to that of an oral tablet containing buprenorphine. Buprenorphine or its salts can be present in the individual film dosage form in an amount of from about 0.5 mg to about 30 mg per dosage, for example, at a 0.5 mg, 0.75 mg, 1 mg, 1.5 mg, 2 mg, 2.5 mg, 3 mg, 3.5 mg, 4 mg, 4.5 mg, 5 mg, 5.5 mg, 7.5 mg, 8 mg, 8.5 mg, 9 mg, 10 mg, 11 mg, 20 mg, 22 mg, 25 mg, or 30 mg dosage including greater than 1 mg, more than 5 mg, more than 20 mg, more than 25 mg, or less than 30 mg, less than 20 mg, less than 15 mg, less than 10 mg, less than 8 mg, less than 5 or less than 3 mg, or any combination thereof. About 10% to about 50%, about 20% to about 40%, about 10%, about 20%, about 30%, about 40%, or about 45% less buprenorphine may be required in the self-supporting film dosage unit than in the corresponding oral tablet to achieve substantially equivalent Cmax, AUC, and Tmax values, i.e., within the range of about 80% to about 125%, at a confidence level of at least about 80%.

In another example, a self-supporting individual film dosage form containing buprenorphine and naloxone has a substantially equivalent pharmacokinetic profile to that of an oral tablet containing buprenorphine. Buprenorphine or its salts can be present in the individual film dosage form in any of the amounts discussed above. Naloxone or its salts can be present in the individual film dosage form in an amount of from about 0.05 mg to about 30 mg per dosage, for example, at 0.1 mg, 0.2 mg, 0.3 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 7.5 mg, 8 mg, 8.5 mg, 9 mg, 10 mg, 11 mg, 20 mg, 22 mg, 25 mg, or 30 mg dosage including greater than 1 mg, more than 5 mg, more than 20 mg, more than 25 mg, or less than 30 mg, less than 20 mg, less than 15 mg, less than 10 mg, less than 8 mg, less than 5, less than 3 mg, less than 2 mg, less than 1 mg, less than 0.5 mg or any combination thereof. About 10% to about 50%, about 20% to about 40%, about 10%, about 20%, about 30%, about 40%, or about 45% less buprenorphine and naloxone may be required in the self-supporting film dosage unit than in the corresponding oral tablet to achieve substantially equivalent Cmax, AUC, and Tmax values, i.e., within the range of about 80% to about 125%, at a confidence level of at least about 80%.

In another example, a self-supporting individual film dosage form including alprazolam, diazepam or epinephrine can have a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a hydrophobic penetration enhancer; (viii) sodium or a salicylic acid derivative; (ix.) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; or (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for as increased bioavailability of the compound in a blood plasma of a subject. The formulation can include approximately the same active pharmaceutical ingredient (API):enhancer ratio as in the other examples for diazepam and alprazolam.

Another pharmaceutical active acceptable for use herein is lumateperone, as disclosed in U.S. Pat. Nos. 9,745,300, 9,708,322, 7,183,282, 7,071,186, 6,552,017, 8,648,077, 8,598,119, 9,751,883, 9,371,324, 9,315,504, 9,428,506, 8,993,572, 8,309,722, 6,713,471, 8,779,139, 9,168,258, RE039680E1, 9616061, 9586960, and in U.S. Patent Publication Nos. 2017114037, 2017183350, 2015072964, 2004034015, 2017189398, 2016310502, 2015080404, the aforementioned contents of which are incorporated by reference herein in their entirety.

The active in the present disclosure may be incorporated into the polymer matrix or film in a controlled release form. For example, particles of active may be coated with polymers such as ethyl cellulose or polymethacrylate, commercially available under brand names such as Aquacoat ECD and Eudragit E-100, respectively. Solutions of active may also be absorbed on such polymer materials and incorporated into the inventive films. Other components such as fats and waxes, as well as sweeteners and/or flavors may also be employed in such controlled release compositions.

The active may be taste-masked prior to incorporation into the polymer matrix or film, as set forth in the PCT Application No. PCT/US02/32594, entitled Uniform Films For Rapid Dissolve Dosage Form Incorporating Taste-Masking Compositions, based on U.S. Provisional Application No. 60/414,276 of the same title, filed Sep. 27, 2002, the entire subject matter of which is incorporated by reference herein.

An anti-oxidant may also be added to the film to prevent the degradation of the active, especially where the active is oxidation sensitive, for example, photosensitive.

Cosmetic active agents may include breath freshening compounds like menthol, other flavors; such as mint, cherry, lemon lime, mixed berry, or grapefruit, or fragrances, especially those used for oral hygiene, as well as actives used in dental and oral cleansing such as quaternary ammonium bases. The effect of flavors may be enhanced using flavor enhancers like tartaric acid, vanillin, or the like.

As disclosed in U.S. Patent Publication No. 2017/0035689, which is incorporated by reference herein in its entirety, when the dosage form includes at least one antagonist in addition to the agonist, it may be desired to control the release of the antagonist, so as to minimize or wholly prevent the absorption of the antagonist from the dosage form when taken orally. In this fashion, the antagonist may be released faster and a larger proportion of it may be present as the ionized form in solution, thereby lessening the likelihood of its absorption in the body. In a dosage form that is placed in the oral cavity, the agonist may be absorbed buccally, so as to provide rapid absorption of the agonist into the body of the patient. At the same time, it may be desired to inhibit or reduce absorption of any antagonist buccally, thereby allowing the antagonist to be swallowed and destroyed in the stomach, or in some cases absorbed in the colon. It may be desired to reduce the absorption of the antagonist by chemical means, such as by controlling the local pH of the dosage form.

It has been found that by controlling the local pH of the dosage form, the release and/or absorption of the active(s) therein may be controlled. For example, in a dosage that includes an amount of an agonist, the local pH may be controlled to a level that optimizes its release and/or absorption into the oral cavity of the patient. In dosages incorporating an agonist and an antagonist, the local pH may be controlled to a level that maximizes the release and/or oral absorption of the agonist while simultaneously minimizing the release and/or oral absorption of the antagonist. For example, the film dosage may include distinct regions, one region including an agonist and the other region including an antagonist, where the local pH of each region is optimized for the desired effect.

The dosage form for mucosal delivery may include a combination of an agonist (or partial agonist) and an antagonist. The film dosage form may include a combination of an agonist and an antagonist, while the dosage has a controlled local pH. The present disclosure is not limited to the use of any one particular agonist and/or antagonist. Any agonist (or partial agonist) and any antagonist may be incorporated herein. The agonist and optional antagonist should be selected from those agonists and antagonists that are useful in treating the particular symptom being treated. The dosage forms for mucosal delivery, including the inventive films discussed, may incorporate agonists and/or antagonists that are basic in nature. Suitable agonists (and/or partial agonists) may include buprenorphine (pKa=8.42), sufentanil (pKa=8.0), morphine (pKa=8.0), fentanil (pKa=8.4), alfentanil (pKa=6.5), pethidine (pKa=8.7), apomorphine (pKa=8.9), alphaprodine (pKa=8.7), remifentanil (pKa=7.0), methadone (pKa=9.2), codeine (pKa=8.2), dihydrocodeine (pKa=9.4), morphine (pKa=8.0), oxycodone (pKa=8.53), oxymorphone (pKa=8.17), tramadol (pKa=9.41), or pharmaceutically acceptable salts thereof. Suitable antagonists (and/or partial antagonists) may include naloxone, maltrexone, nalorphine and levallorphan, or therapeutically acceptable salts thereof.

As discussed above, the local pH of the film is preferably controlled to provide the desired release and/or absorption of the agonist and antagonist. Suitable agonists may have a pKa of about 5 to about 9.5, and most preferably from about 8.0 to about 9.0. Suitable antagonists may have a pKa of about 6.0 to about 9.0, and most preferably about 7.0 to about 9.0. For example, naloxone has a pKa of about 7.94.

In one embodiment, the self-supporting film includes a polymer matrix, a therapeutically effective amount of an agonist or a pharmaceutically acceptable salt thereof, and a buffer. The agonist may be a partial agonist, or an opioid agonist, such as buprenorphine. The buffer is preferably capable of providing a local pH of the composition within a range that provides a controllable level and desirably an optimal treatment level of absorption of the agonist. For example, it may be desired to provide an absorption of buprenorphine that is bioequivalent to a Suboxone® tablet.

Certain agonists, such as buprenorphine, are capable of being suitably absorbed when the local pH of the film composition is either between about 3 to about 4 or between about 5 to about 9. Thus, the local pH for the film including the agonist may be either from about 3 to about 4 or from about 5 to about 9. To provide a maximum absorption of buprenorphine, for example, the local pH of the film may be about 5.5. To provide an absorption of buprenorphine that is bioequivalent to the Suboxone® tablet, the local pH of the film may be about 6 to about 7. The resulting dosage is a film that allows for a rapid and effective release of the agonist (such as buprenorphine) into the oral cavity of the user. At the same time, the film desirably has a sufficient adhesion profile, such that the film cannot easily be removed, or cannot be removed at all, from the oral cavity of the user once it has been placed into the cavity. Full release of the agonist may take place within less than about thirty minutes, e.g., within about 10 minutes to about 30 minutes and preferably remains in the oral cavity for at least 1 minute and desirably about 1 to about 30 minutes.

It may be desirable to combine the opioid agonist (or partial agonist) in the film composition with an opioid antagonist or a pharmaceutically acceptable salt thereof. The agonist and antagonist may be dispersed throughout the dosage separately or the agonist and antagonist may be separately dispersed in individual film regions. Most desirably the antagonist includes naloxone, but any suitable antagonist may be selected as desired. The antagonist may optionally be water-soluble, so as to render separation of the antagonist and agonist difficult, thereby lessening the potential for diversion abuse of the agonist.

As with a film including an agonist, a film including an agonist and an antagonist is desirably pH-controlled through the inclusion of a buffer. At the desired local pH level of the agonist and the antagonist, optimal absorption of the agonist may be achieved while the absorption of the antagonist may be greatly inhibited.

The film may contain any desired level of self-supporting film forming polymer, such that a self-supporting film composition is provided. In one embodiment, the film contains a polymer matrix in an amount of at least about 25% by weight of the film composition. The polymer matrix may be present in an amount of at least about 50% by weight of the film composition, or in a range of about 25% to about 75%, or about 30% to about 50% by weight of the film composition.

Any desired level of agonist and optional antagonist may be included in the dosage, so as to provide the desired therapeutic effect. In certain embodiments, the film composition. includes about 2 mg to about 16 mg, or about 4 mg to about 12 mg, of agonist per dosage. The film composition may include about 0.5 mg to about 5 mg, or about 1 mg to about 3 mg of antagonist per dosage. If an antagonist is incorporated into the film, the film composition may include the antagonist in a ratio of about 6:1-2:1 agonist to antagonist. Most desirably, the film composition contains about 4:1 agonist to antagonist per dosage. For example, in one embodiment, the dosage includes an agonist in an amount of about 12 mg, and includes an antagonist in an amount of about 3 mg.

The film may include at least one buffer so as to control the local pH of the film. Any desired level of buffer may be incorporated into the film so as to provide the desired local pH level. The buffer is preferably provided in an amount sufficient to control the release from the film and/or the absorption into the body of the agonist and the optional antagonist. In a desired embodiment, the film includes buffer in a ratio of buffer to agonist in an amount of from about 2:1 to about 1:5 (buffer:agonist). The buffer may alternatively be provided in a 1:1 ratio of buffer to agonist. A film including an antagonist preferably has a local pH of about 2 to about 4. Any buffer may be used as desired. In some embodiments, the buffer may include Sodium citrate, citric acid. Succinic acid, malic acid, phosphoric acid, boric acid, and combinations thereof. The buffer may include a buffering system including a combination of components, such as Citric Acid/Sodium Citrate, Succinic Acid/Monosodium Succinate, Glycine/SodiumGlycine, Malic Acid/Sodium Malate, Phosphoric Acid/Sodium Phosphate, Fumaric Acid/Sodium Fumarate, Monosodium Phosphate/Disodium Phosphate, and Boric Acid/Sodium Borate.

In this embodiment, the film includes a polymer matrix, an agonist, and an optional antagonist, while the film has a controlled local pH to the level desired. The buffer is desirably present in an amount to provide a therapeutically adequate absorption of the agonist, while simultaneously limiting or preventing substantial absorption of the antagonist. Controlling of the local pH allows for the desired release and/or absorption of the components, and thus provides a more useful and effective dosage.

The film dosage composition may include a polymer matrix, a therapeutically effective amount of agonist, a therapeutically effective amount of antagonist, and a buffering system. A “therapeutically effective amount” of an antagonist is intended to refer to an amount of the antagonist that is useful in diverting abuse of the agonist by a patient. The buffering system may include a buffer in addition to a solvent. The buffering system desirably includes a sufficient level of buffer so as to provide a desired local pH level of the film.

Biological macromolecule actives fall into four major classes: proteins (including amino acids and enzymes), carbohydrates, nucleic acids (e.g., DNA and RNA, including nucleosides and nucleotides), and lipids. The biological macromolecule active used herein may be a protein, macromolecular carbohydrate, glycoprotein, proteoglycan, lignin, biological poly-acid, or a nucleic acid. The protein may be an enzyme such as an apoenzyme or an isoenzyme.

The biological macromolecule active may be synthetic or semi-synthetic. The biological macromolecule active may be or a natural product. Isolated biological macromolecules may be used, for example, in identifying genetic defects, diagnosing diseases, development of new drugs or treatments, and studying gene expression. Purified nucleic acids are derived from biological material samples, such as whole blood, plasma, blood serum, urine, feces, saliva, sperm, tissue, cells, and other body fluids, materials, or plant tissue.

The biological macromolecule active used in the films disclosed herein may be any FDA-approved biologic drug, such as, but not limited to: abciximab (ReoPro), alteplase (Cathflo Activase, Activase), reteplase (Retavase), tenecteplase (TNKase), abobotulinumtoxinA (Dysport), onabotulinumtoxinA (Botox), incobotulinumtoxinA (Xeomin), anakinra (Kineret), rimabotulinumtoxinB (Myobloc), follitropin alpha (Gonal f), abobotulinumtoxinA (Dysport), onabotulinumtoxinA (Botox), collagenase (Santyl), ecallantide (Kalbitor), incobotulinumtoxinA (Xeomin), collagenase Clostridium histolyticum (Xiaflex), aflibercept (Eylea), ranibizumab (Lucentis), incobotulinumtoxinA (Xeomin), ocriplasmin (Jetrea), bevacizumab (Avastin), pegaptanib (Macugen), abatacept (Orencia), adalimumab (Humira), adalimumab-atto (Amjevita), certolizumab pegol (Cimzia), etanercept (Enbrel), etanercept-szzs (Erelzi), golimumab (Simponi), golimumab injection (Simponi Aria), infliximab (Remicade), infliximab-dyyb (Inflectra), secukinumab (Cosentyx), tocilizumab (Actemra), ustekinumab (Stelara), anakinra (Kineret), canakinumab (Ilaris), golimumab injection (Simponi Aria), infliximab (Remicade), infliximab-dyyb (Inflectra), rituximab (rituxan), tocilizumab (Actemra), ustekinumab (Stelara), interferon beta-1b (Betaseron), interferon beta-1b (Extavia), daclizumab (Zenapax), daclizumab (Zinbryta), interferon beta-1a (Avonex), natalizumab (Tysabri), interferon beta-1a (Rebif), peginterferon beta-1a (Plegridy), ixekizumab (Taltz), brodalumab (Siliq), Rilonacept (Arcalyst), belimumab (Benlysta), interferon gamma-1b (Actimmune), asparaginase (Elspar), asparaginase Erwinia chrysanthemi (Erwinaze), blinatumomab (Blincyto), interferon alfa-2b (Intron A), obinutuzumab (Gazyva), ofatumumab (Arzerra), pegaspargase (Oncaspar), sargramostim (Leukine), nivolumab (Opdivo), brentuximab vedotin (Adcetris), ibritumomab tiuxetan (Zevalin), rituximab (Rituxan), ado-trastuzumab emtansine (Kadcyla), pertuzumab (Perjeta), trastuzumab (Herceptin), aldesleukin (Proleukin), daratumumab (Darzalex), elotuzumab (Empliciti), pembrolizumab (Keytruda), ipilimumab (Yervoy), atezolizumab (Tecentriq), cetuximab (Erbitux), necitumumab (Portrazza), nivolumab (Opdivo), ramucirumab (Cyramza), cetuximab (Erbitux), panitumumab (Vectibix), ziv-aflibercept (Zaltrap), capromab pendetide (ProstaScint), trastuzumab (Herceptin), bevacizumab (Avastin), olaratumab (Lartruvo), elosulfase alfa (Vimizim), idursulfase (Elaprase), iaronidase (Aldurazyme), asfotase alfa (Strensiq), pegloticase (Krystexxa), hypercholesterolemia, alirocumab (Praluent), evolocumab (Repatha), albiglutide (Tanzeum), dulaglutide (Trulicity), becaplermin (Regranex), agalsidase beta (Fabrazyme), alglucosidase alfa (Myozyme, Lumizyme), canakinumab (Ilaris), galsulfase (Naglazyme), metreleptin (Myalept), rasburicase (Elitek), sebelipase alfa (Kanuma), parathyroid hormone (Natpara), bezlotoxumab (Zinplava), interferon alfa-n3 (Alferon N Injection), peginterferon alfa-2a (Pegasys), peginterferon alfa-2b (PegIntron, Sylatron), palivizumab (Synagis), siltuximab (Sylvant), pegfilgrastim (Neulasta), epoetin alfa (Epogen/Procrit), methoxy polyethylene glycol-epoetin beta (Mircera), basiliximab (Simulect), belatacept (Nulojix), palifermin (Kepivance), eculizumab (Soliris), oprelvekin (Neumega), romiplostim (Nplate), glucarpidase (Voraxaze), idarucizumab (Praxbind), obiltoxaximab (Anthim), Raxibacumab, dornase alfa (Pulmozyme), mepolizumab (Nucala), reslizumab (Cinqair), and omalizumab (Xolair).

Other suitable actives for use herein are disclosed in U.S. Patent Publication No. 2015/0190476, which is incorporated by reference herein in its entirety.

### Pharmacokinetic Profile

A pharmacokinetic profile is the measure of the movement of a pharmaceutical active into, through, and out of the body, including the time course of its absorption, bioavailability, distribution, metabolism, and excretion. Means of measuring the pharmacokinetic profile include Cmax, AUC and Tmax. Cmax or “Cmax” as used herein means the maximum observed plasma concentration. Tmax or “Tₘₐₓ” as used herein means the time point of maximum observed plasma concentration. Tmax is the time at which Cmax is attained.

The term “AUC” as used herein means “area under the curve” in a plot of concentration of drug in plasma versus time. AUC is usually given for the time interval zero to infinity (AUC_{inf}), however, clearly plasma drug concentrations cannot be measured ‘to infinity’ for a patient so mathematical approaches are used to estimate the AUC from a limited number of concentration measurements (AUCₜ). In a practical sense, the AUC (from zero to infinity) represents the total amount of drug absorbed by the body, irrespective of the rate of absorption. This is useful when trying to determine whether two formulations of the same dose release the same dose of drug to the body. The AUC of a transmucosal dosage form compared to that of the same dosage administered intravenously serves as the basis for a measurement of bioavailability.

A pharmacokinetic profile of two dosage forms containing the same active that is “substantially equivalent” as used herein means that the relative mean Cmax and AUC between the two is within about 75% to about 130% at a confidence level of at least about 80% or greater.

In an embodiment, the pharmacokinetic profile of the film dosage form (pKP_{film}) is about 80% to about 125%, or about 90% to about 115% of the pharmacokinetic profile of the corresponding enterally delivered dosage form (pKP_{ingestible}) at a confidence level of at least about 80%, at least about 70%, or at least about 60%. In another embodiment, the pharmacokinetic profile of the film dosage form (pKP_{film}) is about 95% to about 110% of the pharmacokinetic profile of the corresponding enterally delivered dosage form (pKP_{ingestible}) at a confidence level of at least about 80%, at least about 70%, or at least about 60%.

When the active is a small molecule, one or both of the AUC and Cmax of the self-supporting film dosage form is about 75% to about 130%, about 80% to about 130%, about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the AUC and/or Cmax of the corresponding enterally delivered dosage form. When the active is a small molecule, the Tmax of the self-supporting film dosage form is about 75% to about 130%, about 80% to about 130%, about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the Tmax of the corresponding enterally delivered dosage form. For example, the AUC of the self-supporting film dosage form is about 80% to about 125%, at a confidence level of at least about 80%, of the AUC of the corresponding enterally delivered dosage form, and/or the Cmax of the self-supporting film dosage form is about 80% to about 125%, at a confidence level of at least about 80%, of the Cmax of the corresponding enterally delivered dosage form, and/or the Tmax of the self-supporting film dosage form is about 80% to about 125%, at a confidence level of at least about 80%, of the Tmax of the corresponding enterally delivered dosage form.

When the active is a biological macromolecule, one or both of the AUC and Cmax of the self-supporting film dosage form is about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the AUC and Cmax of the corresponding intravenously delivered dosage form. When the active is a biological macromolecule, the Tmax of the self-supporting film dosage form is about 75% to about 130%, about 80% to about 130%, about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the Tmax of the corresponding intravenously delivered dosage form. For example, the AUC of the self-supporting film dosage form is about 80% to about 125%, at a confidence level of at least about 80%, of the AUC of the corresponding intravenously delivered dosage form, and/or the Cmax of the self-supporting film dosage form is about 80% to about 125%, at a confidence level of at least about 80%, of the Cmax of the corresponding intravenously delivered dosage form, and/or the Tmax of the self-supporting film dosage form is about 80% to about 125%, at a confidence level of at least about 80%, of the Tmax of the corresponding intravenously delivered dosage form.

In another embodiment, the pharmacokinetic profile of the film dosage form (pKP_{film}) is about 80% to about 125%, or about 90% to about 115% of the pharmacokinetic profile of the corresponding intravenously delivered dosage form (pKP_{ingestible}) at a confidence level of at least about 80%, at least about 70%, or at least about 60%. In another embodiment, the pharmacokinetic profile of the film dosage form (pKP_{film}) is about 95% to about 110% of the pharmacokinetic profile of the corresponding intravenously delivered dosage form (pKP_{ingestible}) at a confidence level of at least about 80%, at least about 70%, or at least about 60%.

In an embodiment, the pharmacokinetic profile of the film dosage form (pKP_{film}) is about 80% to about 125%, or about 90% to about 115% of the pharmacokinetic profile of the corresponding enterally or parenterally delivered non-film dosage form (pKP_{ingestible}) at a confidence level of at least about 80%, at least about 70%, or at least about 60%. In another embodiment, the pharmacokinetic profile of the film dosage form (pKP_{film}) is about 95% to about 110% of the pharmacokinetic profile of the corresponding enterally or parenterally delivered non-film dosage form (pKP_{ingestible}) at a confidence level of at least about 80%, at least about 70%, or at least about 60%.

When the active is a small molecule, one or both of the AUC and Cmax of the self-supporting film dosage form is about 75% to about 130%, about 80% to about 130%, about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the AUC and/or Cmax of the corresponding enterally or parenterally delivered non-film dosage form. When the active is a small molecule, the Tmax of the self-supporting film dosage form is about 75% to about 130%, about 80% to about 130%, about 80% to about 125%, about 85% to about 120%, about 90% to about 115%, about 90% to about 110%, or about 95% to about 110%, at a confidence level of at least about 60%, at least about 70%, at least about 80%, at least about 85%, at least about 90%, at least about 95%, or at least about 98%, of the Tmax of the corresponding enterally or parenterally delivered non-film dosage form. For example, the AUC of the self-supporting film dosage form is about 80% to about 125%, at a confidence level of at least about 80%, of the AUC of the corresponding enterally or parenterally delivered non-film dosage form, and/or the Cmax of the self-supporting film dosage form is about 80% to about 125%, at a confidence level of at least about 80%, of the Cmax of the corresponding enterally or parenterally delivered non-film dosage form, and/or the Tmax of the self-supporting film dosage form is about 80% to about 125%, at a confidence level of at least about 80%, of the Tmax of the corresponding enterally or parenterally delivered non-film dosage form.

### Penetration Enhancer

Any penetration enhancer known for use in the art may be incorporated into the dosage forms of the present disclosure. The term “penetration enhancer” is interchangeable with absorption enhancer. When delivered to the mouth via a film, a penetration enhancer is a component that can improve the permeability of the pharmaceutical active through the mucosa and into the blood stream of the subject.

The penetration enhancer may a nonionic alkyl glycoside having a hydrophobic alkyl group joined by a linkage to a hydrophilic saccharide. The penetration enhancer may be selected from the group consisting of a maltoside or maltoside derivative, a sucroside or sucroside derivative, and an essential oil or a component of an essential oil. The penetration enhancer may be selected from the group consisting of alkyl thiomaltoside, maltoside, maltotrioside, maltopyranoside, dodecyl maltoside, tridecyl maltoside, tetradecyl maltoside, tetradecyl-β-D-maltoside, dodecyl-β-D-maltoside, tridecyl-β-D-maltoside, sucroside, sucrose mono-dodecanoate, sucrose mono-tridecanoate, sucrose mono-tetradecanoate and combinations thereof.

The permeation enhancer can improve absorption rate and amount of the pharmaceutical active by more than 5%, more than 10%, more than 20%, more than 30%, more than 40%, more than 50%. more than 60%, more than 70%, more than 80%, more than 90%, more than 100%, more than 150%, about 200% or more, or less than 200%, less than 150%, less than 100%, less than 90%, less than 80%, less than 70%, less than 60%, less than 50%, less than 40%, less than 30%, less than 20%, less than 10%, or less than 5%, or a combination of these ranges, depending on the other components in the composition.

In certain embodiments, the self-supporting film comprises a pharmaceutically acceptable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof; (xiv) an N-acetylamino acid or salt thereof; (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced transmucosal delivery, wherein the formulation of the compound with the transmucosal delivery-enhancing agents provides for increased bioavailability of the compound in blood plasma of a subject. Penetration enhancers have been described in J. Nicolazzo, et al., J. of Controlled Disease, 105 (2005) 1-15, which is incorporated by reference herein.

Surfactants and bile salts have been shown to enhance the permeability of various compounds across the buccal mucosa, both in vitro and in vivo. The data obtained from these studies strongly suggest that the enhancement in permeability is due to an effect of the surfactants on the mucosal intercellular lipids.

Fatty acids have been shown to enhance the permeation of a number of drugs through the skin, and this has been shown by differential scanning calorimetry and Fourier transform infrared spectroscopy to be related to an increase in the fluidity of intercellular lipids.

Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azone® on the permeability of compounds through oral mucosa. Further, chitosan, a biocompatible and biodegradable polymer, has been shown to enhance drug delivery through various tissues, as including the intestine and nasal mucosa.

It has been shown that buccal penetration can be improved by using various classes of transmucosal and transdermal penetration enhancers such as bile salts, surfactants, fatty acids and their derivatives, chelators, cyclodextrins and chitosan. Among these chemicals used for the drug permeation enhancement, bile salts are the most common.

In vitro studies on enhancing effect of bile salts on the buccal permeation of compounds is discussed in Sevda Senel, Drug permeation enhancement via buccal route: possibilities and limitations, Journal of Controlled Release 72 (2001) 133-144, which is incorporated by reference herein. That article also discusses recent studies on the effects of buccal epithelial permeability of dihydroxy bile salts, sodium glycodeoxycholate (SGDC) and sodium taurodeoxycholate (TDC) and tri-hydroxy bile salts, sodium glycocholate (GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, or safrole, or a combination thereof. The phytoextract can be an essential oil extract of a clove plant, for example, from the leaf, stem or flower bud of a clove plant. The clove plant can be Syzygium aromaticum. The phytoextract can include about 20 to about 95% eugenol, including about 40 to about 95% eugenol, including about 60 to about 95% eugenol, and for example, about 80-95% eugenol. The extract can also include about 5% to about 15% eugenol acetate. The extract can also include caryophyllene. The extract can also include up to about 2.1% α-humulen. Other volatile compounds included in lower concentrations in clove essential oil can be β-pinene, limonene, farnesol, benzaldehyde, 2-heptanone or ethyl hexanoate. Other permeation enhancers may be added to the composition to improve absorption of the drug. Suitable permeation enhancers include natural or synthetic bile salts such as sodium fusidate; glycocholate or deoxycholate and their salts; fatty acids and derivatives such as sodium laurate, oleic acid, oleyl alcohol, monoolein, or palmitoylcarnitine; chelators such as disodium EDTA, sodium citrate and sodium laurylsulfate, atone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol. The permeation enhancer can include phytoextract derivatives and/or monolignols. The permeation enhancer can also be a fungal extract.

Some natural products of plant origin have been known to have a vasodilatory effect. For review, see McNeill J. R. and Jurgens, T. M., Can. J. Physiol. Pharmacol. 84:803-821 (2006), which is incorporated by reference herein. Specifically, vasorelaxant effects of eugenol have been reported in a number of animal studies. See, e.g., Lahlou, S., et at., J. Cardiovasc. Pharmacol. 43:250-57 (2004), Damiani, C. E. N., et al., Vascular Pharmacol. 40:59-66 (2003), Nishijima, H., et al., Japanese J. Pharmacol. 79:327-334 (1998), and Hume W. R., J. Dent Res. 62(9):1013-15 (1983), each of which is incorporated by reference herein. Calcium channel blockade was suggested to be responsible for vascular relaxation induced by a plant essential oil, or its main constituent, eugenol. See, Interaminense L. R. L. et al., Fundamental & Clin. Pharmacol. 21: 497-506 (2007), which is incorporated by reference herein.

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference herein. There are two families of essential fatty acids that are metabolized in the human body: Ω-3 and Ω-6 polyunsaturated fatty acids (PUFAs). If the first double bond is found between the third and the fourth carbon atom from the .omega. carbon, they are called Ω-3 fatty acids. If the first double bond is between the sixth and seventh carbon atom, they are called Ω-6 fatty acids. PUFAs are further metabolized in the body by the addition of carbon atoms and by desaturation (extraction of hydrogen). Linoleic acid, which is a Ω-6 fatty acid, is metabolized to γ-linolenic acid, dihomo-γ-linolinic acid, arachidonic acid, adrenic acid, tetracosatetraenoic acid, tetracosapentaenoic acid and docosapentaenoic acid. α-linolenic acid, which is Ω-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na.sup.+K.sup.+-APTase pump and the fatty acids with increasing degrees of cis-unsaturation had higher potencies. See, Pomposiello, S. I. et al., Hypertension 31:615-20 (1998), which is incorporated by reference herein. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Peddersen, C. O. et al., J. Appl. Physiol. 68(5):1799-808 (1990); and see, Sparwhake, E. W., et al., J. Appl. Physiol. 44:397-495 (1978) and Wicks, T. C. et al., Circ. Res, 38:167-71 (1976), each of which is incorporated by reference herein.

Many studies have reported effects of eicosapentaenoic acid (EPA) and docosahexaenoic acid (DHA) on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F., et al., Hypertension 21:22-8 (1993), and Tagawa, H, et al., J Cardiovasc Pharmacol 33:633-40 (1999), each of which is incorporated by reference herein. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference herein. Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50(4):164-70 (2007), which is incorporated by reference herein.

The adrenergic receptors (or adrenoceptors) are a class of G protein-coupled receptors that are a target of catecholamines, especially norepinephrine (noradrenaline) and epinephrine (adrenaline). Epinephrine (adrenaline) interacts with both α- and β.-adrenoceptors, causing vasoconstriction and vasodilation, respectively. Although αreceptors are less sensitive to epinephrine, when activated, they override the vasodilation mediated by β-adrenoceptors because there are more peripheral α1 receptors than β-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The α1-adrenoreceptor is known for smooth muscle contraction, mydriasis, vasoconstriction in the skin, mucosa and abdominal vicera and sphincter contraction of the gastrointestinal (GI) tract and urinary bladder. The α1-adrenergic receptors are member of the G_{q} protein-coupled receptor superfamily. Upon activation, a heterotrimeric G protein, G_{q}, activates phospholipase C (PLC). The mechanism of action involves interaction with calcium channels and changing the calcium content in a cell. For review, see Smith R. S. et al., Journal of Neurophysiology, 102(2): 1103-14 (2009), which is incorporated by reference herein. Many cells possess these receptors.

α 1-adrenergic receptors can be a main receptor for fatty acids. For example, saw palmetto is extract (SPE), widely used for the treatment of benign prostatic hyperplasia (BPH), has been reported to bind .alpha.1-adrenergic, muscarinic and 1,4-dihydropyridine (1,4-DH P) calcium channel antagonist receptors. See, Abe M., et al., Biol. Pharm. Bull. 32(4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to a 1-adrenergic, muscarinic and 1,4-DHP calcium channel antagonist receptors.

In certain embodiments, a permeation enhancer can be an adrenergic receptor interacter. An adrenergic receptor interacter refers to a compound or substance that modifies and/or otherwise alters the action of an adrenergic receptor. For example, an adrenergic receptor interacter can prevent stimulation of the receptor by increasing, or decreasing their ability to bind. Such interacters can be provided in either short-acting or long-acting forms. Certain short-acting interacters can work quickly, but their effects last only a few hours. Certain long-acting interacters can take longer to work, but their effects can last longer. The interacter can be selected and/or designed based on, e.g., one or more of the desired delivery and dose, active pharmaceutical ingredient, permeation modifier, permeation enhancer, matrix, and the condition being treated. An adrenergic receptor interacter can be an adrenergic receptor blocker. The adrenergic receptor interacter can be a terpene (e.g. volatile unsaturated hydrocarbons found in the essential oils of plants, derived from units of isoprenes) or a C3-C22 alcohol or acid, preferably a C7-C18 alcohol or acid. In certain embodiments, the adrenergic receptor interacter can include farnesol, linoleic acid, arachidonic acid, docosahexanoic acid, eicosapentanoic acid, and/or docosapentanoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

The C3-C22 alcohol or acid can be an alcohol or acid having a straight C3-C22 hydrocarbon chain, for example a C3-C22 hydrocarbon chain optionally containing at least one double bond, at least one triple bond, or at least one double bond and one triple bond; said hydrocarbon chain being optionally substituted with C₁₋₄ alkyl, C₂₋₄ alkenyl, C₂₋₄ alkynyl, C₁₋₄ alkoxy, hydroxyl, halo, amino, nitro, cyano. C₃₋₅ cycloalkyl, 3-5 membered heterocycloalkyl, monocyclic aryl, 5-6 membered heteroaryl, C₁₋₄ alkylcarbonyloxy, C₁₋₄ alkylcyloxycarbonyl, C₁₋₄ alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl.

Fatty acids with a higher degree of unsaturation are effective candidates to enhance the permeation of drugs. Unsaturated fatty acids showed higher enhancement than saturated fatty acids, and the enhancement increased with the number of double bonds. See, A. Mittal, et al, Status of Fatty Acids as Skin Penetration Enhancers—A Review, Current Drug Delivery, 2009, 6, pp. 274-279, which is incorporated by reference herein. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than a fatty acid which has double bond at an odd number position. Cis-unsaturation in the chain can tend to increase activity.

An adrenergic receptor interacter can be a terpene. Hypotensive activity of terpenes in essential oils has been reported. See, Menezes I. A. et al., Z. Naturforsch. 65c:652-66 (2010), which is incorporated by reference herein. In certain embodiments, the permeation enhancer can be a sesquiterpene. Sesquiterpenes are a class of terpenes that consist of three isoprene units and have the empirical formula C₁₅H₂₄. Like monoterpenes, sesquiterpenes may be acyclic or contain rings, including many unique combinations. Biochemical modifications such as oxidation or rearrangement produce the related sesquiterpenoids.

An adrenergic receptor interacter can be an unsaturated fatty acid such as linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in perfumes and flavors. It has been reported that farnesol selectively kills acute myeloid leukemia blasts and leukemic cell lines in preference to primary hemopoietic cells, See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both Farnesol and N-acetyl-S-trans, trans-famesyl-L-cysteine (AFC), a synthetic mimic of the carboxyl terminus of farnesylated proteins inhibited vasoconstriction in rat aortic rings.

In an embodiment, the film comprises a permeation enhancer that includes one or more of a phenylpropanoid, farnesol, Labrasol, and linoleic acid. In an embodiment, the permeation enhancer is a phenylpropanoid that is selected from the group consisting of: eugenol; eugenol acetate; a cinnamic acid; a cinnamic acid ester; a cinnamic aldehyde; a hydrocinnamic acid; chavicol; safrole; or a combination thereof.

In an embodiment, the film comprises a permeation enhancer that is a phytoextract. The phytoextract may be an essential oil extract of a clove plant, an essential oil extract of a leaf of a clove plant, an essential oil extract of a flower bud of a clove plant, an essential oil extract of a stem of a clove plant, or a combination thereof. In an embodiment, the phytoextract can be synthetic. The phytoextract may include about 20% to about 95% eugenol, about 40% to about 95% eugenol, about 60% to about 95% eugenol, or about 80% to about 95% eugenol.

### Pharmaceutically Acceptable Film

Another embodiment is a pharmaceutically acceptable film for oral mucosal delivery of a pharmaceutical active comprising the pharmaceutical active and a water-soluble or water swellable film-forming polymer matrix. The terms used in this embodiment have the same meanings as defined above.

The pharmaceutical active may be a small molecule or biological macromolecule. In an embodiment, the pharmaceutical active is rizatriptan, or a salt, prodrug, derivative, analogue, or ester thereof, optionally in combination with an NSAID. In an embodiment, the pharmaceutical active is testosterone. In another embodiment, the pharmaceutical active is diazepam, or a salt, prodrug, derivative, analogue, or ester thereof. In another embodiment, the pharmaceutical active is pregabalin (Lyrica), or a salt, prodrug, derivative, analogue, or ester thereof. In yet another embodiment, the pharmaceutical active is epinephrine, or a salt, prodrug, derivative, analogue, or ester thereof. In a further embodiment, the pharmaceutical active is sildenafil, or a salt, prodrug, derivative, analogue, or ester thereof. In a further embodiment, the pharmaceutical active is clobazam, or a salt, prodrug, derivative, analogue, or ester thereof. In a still further embodiment, the pharmaceutical active is riluzole, or a salt, prodrug, derivative, analogue, or ester thereof. In a further embodiment, the pharmaceutical active is apomorphine, or a salt, prodrug, derivative, analogue, or ester thereof. In yet another embodiment, the pharmaceutical active is buprenorphine, or a salt, prodrug, derivative, analogue, or ester thereof, optionally in combination with naloxone.

The polymer matrix may include a polyethylene oxide composition, water or a water-containing solvent, and silicon dioxide. The silicon dioxide may be present in about 0.02% to about 3%, about 0.02% to about 1%, about 0.1% to about 3%, about 0.1% to about 1%, about 0.05% to about 2%, about 1% to about 3%, about 0.5% to about 2%, or about 0.5% to about 1.5% by weight of the polymer matrix.

A “polyethylene oxide composition” as used herein refers to one or more than one polyethylene oxide, and optionally silicon dioxide. The polyethylene oxide composition may comprise one polyethylene oxide or a mixture of two, three, or four polyethylene oxides of different molecular weights. In an embodiment, the polymer matrix includes a polyethylene oxide composition of two polyethylene oxides. For example, a polyethylene oxide composition may contain a first polyethylene oxide having a molecular weight below 500,000, and a second polyethylene oxide having a molecular weight above 500,000. In an embodiment thereof, the first polyethylene oxide has a molecular weight of about 100,000, 200,000 or 300,000, and the second polyethylene oxide has a molecular weight of about 700,000, 800,000 or 900,000.

In an embodiment, the polymer matrix includes a polyethylene oxide composition of three polyethylene oxides. For example, a polyethylene oxide composition may contain a first polyethylene oxide having a molecular weight below 500,000, a second polyethylene oxide having a molecular weight above 500,000, and a third polyethylene oxide having a molecular weight below 500,000 but greater than the first polyethylene oxide. In an embodiment thereof, the first polyethylene oxide has a molecular weight of about 100,000, 200,000 or 300,000, the second polyethylene oxide has a molecular weight of about 700,000, 800,000 or 900,000, and the third polyethylene oxide has a molecular weight of about 200,000, 300,000 or 400,000.

In an embodiment thereof, the pharmaceutically acceptable film is an individual unit dosage form, each individual unit dosage form contains about 0.5% to about 50% less active than the corresponding enterally delivered dosage form, and, when it is administered to a patient, one or more of the AUC, Cmax or Tmax of the pharmaceutical active is about 80% to about 125% of the AUC, Cmax or Tmax, respectively, of the corresponding enterally delivered dosage form.

In another embodiment thereof, the pharmaceutically acceptable film is an individual unit dosage form, the active is a biological macromolecule, each individual unit dosage form contains about 10% to about 10,000% more active than the corresponding intravenously delivered dosage form, and, when it is administered to a patient, one or more of the AUC, Cmax or Tmax of the pharmaceutical active is about 80% to about 125% of the AUC, Cmax or Tmax, respectively, of the corresponding enterally delivered dosage form.

In another embodiment thereof, the pharmaceutically acceptable film is an individual unit dosage form, each individual unit dosage form contains about 0.5% to about 50% less active than the corresponding enterally or parenterally delivered non-film dosage form, and, when it is administered to a patient, one or more of the AUC, Cmax or Tmax of the pharmaceutical active is about 80% to about 125% of the AUC, Cmax or Tmax, respectively, of the corresponding enterally or parenterally delivered non-film dosage form.

One or more of the AUC, Cmax or Tmax of the pharmaceutical active may be about 80% to about 125%, about 90% to about 115%, or about 95% to about 110% of the AUC, Cmax or Tmax of the corresponding dosage form of a different administration and/or form at a confidence level of at least about 80%, at least about 70%, or at least about 60%.

### Method of Use

Another embodiment is a method of treating a patient comprising administering a self-supporting film or pharmaceutically acceptable film disclosed herein to the patient in need thereof. The terms used in this embodiment have the same meanings as defined above.

### Forming the Film

The self-supporting films of the present disclosure may be formed into a sheet prior to drying. After the desired components are combined to form a multi-component matrix, including the polymer, water, and pharmaceutical active, and other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or in other words able to maintain their integrity and structure in the absence of a separate support. Furthermore, the films of the present invention may use selected materials that are edible or ingestible.

Coating or casting methods are particularly useful for the purpose of forming the films of the present invention. Specific examples include reverse roll coating, gravure coating, immersion or dip coating, metering rod or Mayer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating approaches or is 100% solids, the process is termed “Extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

The present invention yields exceptionally uniform film products when attention is paid to reducing the aggregation of the compositional components. By avoiding the introduction of and eliminating excessive air in the mixing process, selecting polymers and solvents to provide a controllable viscosity and by drying the film in a rapid manner from the bottom up, such films result.

The products and processes of the present invention rely on the interaction among various steps of the production of the films in order to provide films that substantially reduce the self-aggregation of the components within the films. Specifically, these steps include the particular method used to form the film, making the composition mixture to prevent air bubble inclusions, controlling the viscosity of the film forming composition and the method of drying the film. More particularly, a greater viscosity of components in the mixture is particularly useful when the pharmaceutical active is not soluble in the selected polar solvent in order to prevent the pharmaceutical active from settling out. However, the viscosity must not be too great as to hinder or prevent the chosen method of casting, which desirably includes reverse roll coating due to its ability to provide a film of substantially consistent thickness.

In addition to the viscosity of the film or film-forming components or matrix, there are other considerations taken into account by the present invention for achieving desirable film uniformity. For example, stable suspensions are achieved which prevent solid (such as drug particles) sedimentation in non-colloidal applications. One approach provided by the present invention is to balance the density of the particulate (ρₚ) and the liquid phase (ρ₁) and increase the viscosity of the liquid phase (μ). For an isolated particle, Stokes law relates the terminal settling velocity (Vo) of a rigid spherical body of radius (r) in a viscous fluid, as follows:

Vₒ=(2gr^{r})(ρₚ−ρ₁)/9μ

At high particle concentrations, however, the local particle concentration will affect the local viscosity and density. The viscosity of the suspension is a strong function of solids volume fraction, and particle-particle and particle-liquid interactions will further hinder settling velocity.

Stokian analysis has shown that the incorporation of a third phase, dispersed air or nitrogen, for example, promotes suspension stability. Further, increasing the number of particles leads to a hindered settling effect based on the solids volume fraction. In dilute particle suspensions, the rate of sedimentation, v, can be expressed as:

v/Vₒ=1/(1+κφ)

where κ=a constant, and φ is the volume fraction of the dispersed phase. More particles suspended in the liquid phase results in decreased velocity. Particle geometry is also an important factor since the particle dimensions will affect particle-particle flow interactions.

Similarly, the viscosity of the suspension is dependent on the volume fraction of dispersed solids. For dilute suspensions of non-interaction spherical particles, an expression for the suspension viscosity can be expressed as:

μ/μₒ=1+2.5ϕ

where μₒ is the viscosity of the continuous phase and ϕ is the solids volume fraction. At higher volume fractions, the viscosity of the dispersion can be expressed as

μ/μₒ=1+2.5φ+C₁φ²+C₂φ³+

where C is a constant.

The viscosity of the liquid phase is critical and is desirably modified by customizing the liquid composition to a viscoelastic non-Newtonian fluid with low yield stress values. This is the equivalent of producing a high viscosity continuous phase at rest. Formation of a viscoelastic or a highly structured fluid phase provides additional resistive forces to particle sedimentation. Further, flocculation or aggregation can be controlled minimizing particle-particle interactions. The net effect would be the preservation of a homogeneous dispersed phase.

The addition of hydrocolloids to the aqueous phase of the suspension increases viscosity, may produce viscoelasticity, and can impart stability depending on the type of hydrocolloid, its concentration and the particle composition, geometry, size, and volume fraction. The particle size distribution of the dispersed phase needs to be controlled by selecting the smallest realistic particle size in the high viscosity medium, i.e., <500 μm. The presence of a slight yield stress or elastic body at low shear rates may also induce permanent stability regardless of the apparent viscosity. The critical particle diameter can be calculated from the yield stress values. In the case of isolated spherical particles, the maximum shear stress developed in settling through a medium of given viscosity can be given as:

τₘₐₓ=3Vμ/2r

For pseudoplastic fluids, the viscosity in this shear stress regime may well be the zero shear rate viscosity at the Newtonian plateau.

A stable suspension is an important characteristic for the manufacture of a pre-mix composition which is to be fed into the film casting machinery film, as well as the maintenance of this stability in the wet film stage until sufficient drying has occurred to lock-in the particles and matrix into a sufficiently solid form such that uniformity is maintained. For viscoelastic fluid systems, a rheology that yields stable suspensions for extended time period, such as 24 hours, must be balanced with the requirements of high-speed film casting operations. A desirable property for the films is shear thinning or pseudoplasticity, whereby the viscosity decreases with increasing shear rate. Time dependent shear effects such as thixotropy are also advantageous. Structural recovery and shear thinning behavior are important properties, as is the ability for the film to self-level as it is formed.

The rheology requirements for the inventive compositions and films are quite severe. This is due to the need to produce a stable suspension of particles, for example 30-60 wt %, in a viscoelastic fluid matrix with acceptable viscosity values throughout a broad shear rate range. During mixing, pumping, and film casting, shear rates in the range of 10-10⁵ sec.⁻¹ may be experienced and pseudoplasticity is the preferred embodiment.

In film casting or coating, rheology is also a defining factor with respect to the ability to form films with the desired uniformity. Shear viscosity, extensional viscosity, viscoelasticity, and structural recovery will influence the quality of the film. As an illustrative example, the leveling of shear-thinning pseudoplastic fluids has been derived as:

α^{(n-1/n)}=αₒ^{(n-1/n)}−((n−1)/(2n−1))(τ/K)^{1/n}(2π/λ)^{(3+n)/n}h^{(2n+1)/n}t

where α is the surface wave amplitude, αₒ is the initial amplitude, λ is the wavelength of the surface roughness, and both “n” and “K” are viscosity power law indices. In this example, leveling behavior is related to viscosity, increasing as n decreases, and decreasing with increasing K.

Desirably, the films or film compositions of the present disclosure have a very rapid structural recovery, i.e. as the film is formed during processing, it doesn't fall apart or become discontinuous in its structure and compositional uniformity. Such very rapid structural recovery retards particle settling and sedimentation. Moreover, the films or film compositions are desirably shear-thinning pseudoplastic fluids. Such fluids with consideration of properties, such as viscosity and elasticity, promote thin film formation and uniformity.

Thus, uniformity in the mixture of components depends upon numerous variables. As described herein, viscosity of the components, the mixing techniques and the rheological properties of the resultant mixed composition and wet casted film are important aspects of the present invention. Additionally, control of particle size and particle shape is a further consideration. Desirably, the size of the particulate a particle size of 150 microns or less, for example 100 microns or less. Moreover, such particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles.

A number of techniques may be employed in the mixing stage to prevent bubble inclusions in the final film. To provide a composition mixture with substantially no air bubble formation in the final product, anti-foaming or surface-tension reducing agents are employed. Additionally, the speed of the mixture is desirably controlled to prevent cavitation of the mixture in a manner which pulls air into the mix. Finally, air bubble reduction can further be achieved by allowing the mix to stand for a sufficient time for bubbles to escape prior to drying the film. Desirably, the inventive process first forms a masterbatch of film-forming components without active ingredients such as drug particles or volatile materials such as flavor oils. The actives are added to smaller mixes of the masterbatch just prior to casting. Thus, the masterbatch pre-mix can be allowed to stand for a longer time without concern for instability in drug or other ingredients.

In embodiments, the multi-component matrix is a polymer matrix, which is then formed into a sheet as described above. In an embodiment, the polymer matrix is a Non-Newtonian visco-elastic polymer matrix. In one embodiment, the polymer matrix is a shear-thinning pseudoplastic fluid when exposed to shear rates of 10-10⁵ sec⁻¹.

### Drying the Film

The drying step is also a contributing factor with regard to maintaining the uniformity of the film composition. A controlled drying process is particularly important when, in the absence of a viscosity increasing composition or a composition in which the viscosity is controlled, for example by the selection of the polymer, the components within the film may have an increased tendency to aggregate or conglomerate. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se.

When a controlled or rapid drying process is desired, this may be through a variety of methods. A variety of methods may be used including those that require the application of heat. The liquid carriers are removed from the film in a manner such that the uniformity, or more specifically, the non-self-aggregating uniform heterogeneity, that is obtained in the wet film is maintained.

Desirably, the film is dried from the bottom of the film to the top of the film. Desirably, substantially no air flow is present across the top of the film during its initial setting period, during which a solid, visco-elastic structure is formed. The initial setting period, during which a solid, visco-elastic structure is formed, can take place within the first few minutes, e.g. within about the first 4 minutes or about the first 0.5 to about 4.0 minutes of the drying process.

In embodiments, at least a portion of the solvent is rapidly removed from the matrix to form a visco-elastic film having the pharmaceutical active substantially uniformly distributed throughout by rapidly increasing the viscosity of the matrix upon initiation of drying within about 4 minutes to maintain the uniform distribution of the pharmaceutical active by locking-in or substantially preventing migration of the pharmaceutical active.

Controlling the drying in this manner prevents the destruction and reformation of the film's top surface, which results from conventional drying methods. This is accomplished by placing the liquid visco-elastic composition on the top side of a surface having top and bottom sides. Then, heat is initially applied to the bottom side of the visco-elastic film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

In embodiments of the present invention, any top air flow does not overcome the inherent viscosity of the polymer matrix (e.g., the visco-elastic film) and/or any top air flow is insufficient to cause one or more of the following: (i) surface skinning prior to drying the depth of the film, (ii) surface rippling; (iii) self-aggregation of components; (iv) non-uniformity in the thickness of the film, and (v) non-uniformity of mass per unit volume.

The endogenous or internal temperature of the films should be less than about 100° C., desirably about 90° C. or less, and most desirably about 80° C. or less. The temperature inside the drying apparatus (i.e., exogenous to the film) may be any desired temperature and may be well above or below 100° C. In embodiment of the present invention, the differential in temperature between the endogenous or internal temperature and the temperature inside the drying apparatus is at least about 5° C., preferably from about 5° C. to about 30° C.

Another method of controlling the drying process, which may be used alone or in combination with other controlled methods as disclosed above includes controlling and modifying the humidity within the drying apparatus where the film is being dried. In this manner, the premature drying of the top surface of the film is avoided.

Additionally, it has also been discovered that the length of drying time can be properly controlled, i.e. balanced with the heat sensitivity and volatility of the components, and particularly the flavor oils and drugs. The amount of energy, temperature and length and speed of the conveyor can be balanced to accommodate such actives and to minimize loss, degradation or ineffectiveness in the final film.

A specific example of an appropriate drying method is that disclosed by Magoon. Magoon is specifically directed toward a method of drying fruit pulp. However, the present inventors have adapted this process toward the preparation of thin films.

The method and apparatus of Magoon are based on an interesting property of water. Although water transmits energy by conduction and convection both within and to its surroundings, water only radiates energy within and to water. Therefore, the apparatus of Magoon includes a surface onto which the fruit pulp is placed that is transparent to infrared radiation. The underside of the surface is in contact with a temperature controlled water bath. The water bath temperature is desirably controlled at a temperature slightly below the boiling temperature of water. When the wet fruit pulp is placed on the surface of the apparatus, this creates a “refractance window.” This means that infrared energy is permitted to radiate through the surface only to the area on the surface occupied by the fruit pulp, and only until the fruit pulp is dry. The apparatus of Magoon provides the films of the present invention with an efficient drying time reducing the instance of aggregation of the components of the film.

Another method of controlling the drying process involves a zone drying procedure. A zone drying apparatus may include a continuous belt drying tunnel having one or more drying zones located within. The conditions of each drying zone may vary, for example, temperature and humidity may be selectively chosen. It may be desirable to sequentially order the zones to provide a stepped up drying effect.

In aspects of the invention, the speed of the zone drying conveyor desirably is constant. Alternatively, the speed may be altered at a particular stage of the drying procedure to increase or decrease exposure of the film to the conditions of the desired zone. Whether continuous or modified, the zone drying dries the film without surface skinning.

To further control temperature and humidity, the drying zones may include additional atmospheric conditions, such as inert gases. The zone drying apparatus further may be adapted to include additional processes during the zone drying procedure, such as, for example, spraying and laminating processes, so long as controlled drying is maintained in accordance with the invention.

The films may initially have a thickness of about 500 μm to about 1,500 μm, or about 20 mils to about 60 mils, and when dried have a thickness from about 3 μm to about 500 μm, or about 0.1 mils to about 20 mils. Desirably, the dried films will have a thickness of about 1 mils to about 10 mils, more desirably about 2 mils to about 8 mils, and even more desirably, from about 3 mils to about 6 mils.

### Testing Films for Uniformity

It may be desirable to test the films of the present invention for chemical and physical uniformity during the film manufacturing process. In particular, samples of the film may be removed and analytically tested for uniformity in film components between various samples. Film thickness and overall appearance may also be checked for physical uniformity. Active uniform films are desired, particularly for films containing pharmaceutical active components due to safety and efficacy reasons.

A method for testing uniformity in accordance with the present invention includes conveying a film through a manufacturing process. This process may include subjecting the film to drying processes, dividing the film into individual dosage units, and/or packaging the dosages, among others. As the film is conveyed through the manufacturing process, for example on a conveyor belt apparatus, it is cut widthwise into at least one portion. The at least one portion has opposing ends that are separate from any other film portion. For instance, if the film is a roll, it may be cut into separate sub-rolls. Cutting the film may be accomplished by a variety of methods, such as with a knife, razor, laser, or any other suitable means for cutting a film.

The cut film then may be sampled by removing small pieces from each of the opposed ends of the portion(s), without disrupting the middle of the portion(s). Leaving the middle section intact permits the predominant portion of the film to proceed through the manufacturing process without interrupting the conformity of the film and creating sample-inducted gaps in the film. Accordingly, the concern of missing doses is alleviated as the film is further processed, e.g., packaged. Moreover, maintaining the completeness of cut portions or sub-rolls throughout the process will help to alleviate the possibility of interruptions in further film processing or packaging due to quality control issues, for example, alarm stoppage due to notice of missing pieces.

After the end pieces, or sampling sections, are removed from the film portion(s), they may be tested for physical defects in the film and for desired amount of active uniformity in the content of components between samples. Any conventional means for examining and testing the film pieces may be employed, such as, for example, visual inspection, use of analytical equipment, and any other suitable means known to those skilled in the art. Testing for content uniformity of the desired amount of active may be carried out by one or more analytic methods including high pressure liquid chromatography (HPLC) or near-infrared (NIR) spectroscopy. If the testing results show non-uniformity between film samples, the manufacturing process may be altered. For example, the compositional components, compositional rheology, drying conditions, and mixing conditions may be changed. Altering the drying conditions may involve changing the temperature, drying time, film speed through the oven, and dryer positioning, among others.

Moreover, it may be desirable to repeat the steps of sampling and testing throughout the manufacturing process. Testing at multiple intervals may ensure that physically uniform film dosages and film dosages with desired active content uniformity are continuously produced. Alterations to the process can be implemented at any stage to minimize non-uniformity between samples.

In embodiments, the films of the present invention have a substantially uniform content of active by weight per unit volume of the film. In an embodiment, the amount of active in substantially equally sized individual dosage units of the film varies by no more than 10% by weight from a desired amount (e.g., the label claim amount). In another embodiment, the amount of active in substantially equally sized individual dosage units of the film varies by no more than 10% between units.

As used herein, the term desired amount of active component per dosage unit means an amount of active component that is intended to be in each dosage unit. In the case of certain products, e.g., pharmaceutical products, there may be an amount of active component claimed on the label of the product. Thus, a desired active component (or drug) label claim per dosage unit means the amount of active component that is claimed to be in each dosage unit based upon the label of the product.

Uniformity of content of active component in a lot may be determined through establishing the amount of active component (A_{N(i)}) actually present in each sampled individual dosage unit from the same lot (N) as determined by taking the difference between the amount of active component in the sample with the most amount of active component (Max_{LOT(N)}) minus the amount of active component in the sample with the least amount of active component (Min_{LOT(N)}) and dividing the difference by the average amount of active component in the lot samples (Lot_{(N)} Sample Average).

(Max_{LOT(N)}−Min_{LOT(N)})/((A_{N(1)}+A_{N(2)}+++A_{N(10)})/10).

Uniformity of content across different lots may be determined through establishing the amount of active component actually present in each of the sampled individual dosage unit from the different lots and comparing that amount of active component with a desired amount of active component contained therein. The desired amount of active component, when it is a pharmaceutical, may be referred to as the “label claim amount”, thus identifying the amount of pharmaceutical active in the film dosage unit.

In an embodiment, the amount of active in substantially equally sized individual dosage units of the film is not less than 75% or greater than 125% of the desired amount, preferably not less than 85% or greater than 115% of the desired amount.

Using the methods of the present invention, the uniformity of content with respect to desired amount of active is achievable both within individual lots of the film and between different lots of film during large-scale manufacturing of the film. Using the methods of the present invention, this uniformity of content with respect to the amount of active in substantially equally sized individual dosage units is achievable both within individual lots of the film and between different lots of film during large-scale manufacturing of the film.

### Uses of Films

The films of the present invention are well suited for many uses. The high degree of desired active uniformity in the film makes them particularly well suited for incorporating pharmaceuticals. Furthermore, the polymers used in construction of the films may be chosen to allow for a range of disintegration times for the films. A variation or extension in the time over which a film will disintegrate may achieve control over the rate that the active is released, which may allow for a sustained release delivery system. In addition, the films may be used for the administration of a desired amount of a pharmaceutical active to any of several body surfaces, especially those including mucosal membranes, such as those found in the oral (i.e., sublingual, lingual, buccal, and gingival), anal, vaginal, ocular, nasal, aural, ophthalmological, and peritoneal environments; the surface of a wound, either on a skin surface or within the body such as during surgery or left in place after surgery to deliver the desired amount of active after the surgical procedure is completed; the surface of an organ (i.e., kidney, lung, liver, heart, etc.), and other similar surfaces.

The films may be used to orally administer a pharmaceutical active. This is accomplished by preparing the films as described above and introducing them to the oral cavity of an animal, such as a mammal. This film may be prepared and adhered to a second or support layer from which it is removed prior to use, i.e. introduction to the oral cavity. An adhesive may be used to attach the film to the support or backing material which may be any of those known in the art, and is preferably not water-soluble. If an adhesive is used, it will desirably be a food grade adhesive that is ingestible and does not alter the properties of the active. Mucoadhesive compositions are particularly useful. The film compositions in many cases serve as mucoadhesives themselves.

The films may be applied under or to the tongue of the patient, such as a mammal or human, including both an adult human and a child. When this is desired, a specific film shape, corresponding to the shape of the tongue may be preferred. Therefore the film may be cut to a shape where the side of the film corresponding to the back of the tongue will be longer than the side corresponding to the front of the tongue. Specifically, the desired shape may be that of a triangle or trapezoid. Desirably, the film will adhere to the oral cavity preventing it from being ejected from the oral cavity and permitting more of the active to be introduced to the oral cavity as the film dissolves.

Another use for the films of the present invention takes advantage of the films' tendency to dissolve quickly when introduce to a liquid. A pharmaceutical active may be introduced to a liquid by preparing a film in accordance with the present invention, introducing it to a liquid, and allowing it to dissolve. This may be used either to prepare a liquid dosage form of a pharmaceutical active, or to flavor a beverage.

The films of the present invention are desirably packaged in sealed, air and moisture resistant packages to protect the active from exposure oxidation, hydrolysis, volatilization and interaction with the environment. Moreover, the films of the present invention dissolve instantly upon contact with saliva or mucosal membrane areas, eliminating the need to wash the dose down with water.

Desirably, a series of such unit doses are packaged together in accordance with the prescribed regimen or treatment, e.g., a 10-90 day supply, depending on the particular therapy. The individual films can be packaged on a backing and peeled off for use.

The features and advantages of the present invention are more fully shown by the following examples which are provided for purposes of illustration, and are not to be construed as limiting the invention in any way.

### EXAMPLES

### Example 1: Buprenorphine/Naloxone Films

Self-supporting individual film dosage forms including a combination of buprenorphine and naloxone were prepared. Four different strength films were prepared in unit dosages, which include a ratio of buprenorphine to naloxone of 16/4, 12/3, 8/2, and 2/0.5. The compositions are summarized in Table 1 below.

TABLE 1
Compositions of Buprenorphine/Naloxone
Film Unit Dosage Forms

| | Buprenorphine/Naloxone Films | | | |
|---|---|---|---|---|
| | Unit Formula (mg per film strip) | | | |
| | Buprenorphine/Naloxone Ratios | | | |
| Components | 16/4 | 12/3 | 8/2 | 2/0.5 |
| Active Components | | | | |
| Buprenorphine HCl | 17.28 | 12.96 | 8.64 | 2.16 |
| Naloxone HCl Dihydrate | 4.88 | 3.66 | 2.44 | 0.61 |
| Inactive Components | | | | |
| Polyethylene Oxide, NF | 27.09 | 20.32 | 13.55 | — |
| (MW 200,000) | | | | |
| Polyethylene Oxide, NF | 12.04 | 9.03 | 6.02 | 19.06 |
| (MW 100,000) | | | | |
| Polyethylene Oxide, NF | 4.82 | 3.62 | 2.41 | 2.05 |
| (MW 900,000) | | | | |
| Maltitol, NF | 12.04 | 9.03 | 6.02 | 5.87 |
| Flavor | 6.0 | 4.5 | 3.0 | 2.4 |
| Citric Acid, USP | 5.92 | 4.44 | 2.96 | 2.96 |
| HPMC | 4.22 | 3.16 | 2.11 | 2.34 |
| Ace-K | 3.0 | 2.25 | 1.5 | 1.2 |
| Sodium Citrate, anhydrous | 2.68 | 2.01 | 1.34 | 1.34 |
| Colorant | 0.03 | 0.02 | 0.01 | 0.01 |
| Total (mg) | 100 | 75 | 50 | 40 |

These samples were tested for absorption data, including Cmax and AUC absorption levels.

### Example 2: Buprenorphine Films

Self-supporting individual film dosage forms were prepared including buprenorphine. Two different strength film compositions were prepared, which include buprenorphine in a dosage amount of 8 mg and in a dosage amount of 2 mg. The compositions are summarized in Table 2 below.

TABLE 2
Compositions of Buprenorphine Film Unit Dosage Forms

| | |
|---|---|
| | Buprenorphine Film Unit Formula |
| Components | (mg per film strip) |

| | | |
|---|---|---|
| Buprenorphine | 8.64 | 2.16 |
| Inactive Components | | |
| Polyethylene Oxide, NF | 17.66 | 21.87 |
| (MW 100,000) | | |
| Polyethylene Oxide, NF | 2.17 | 2.35 |
| (MW 900,000) | | |
| Maltitol, NF | 5.43 | 6.72 |
| Flavor | 2.8 | 2.8 |
| HPMC | 1.9 | 2.69 |
| Ace-K | 1.2 | 1.2 |
| Colorant | 0.2 | 0.2 |
| Total (mg) | 40 | 40 |

Five additional film dosage compositions were prepared, each including buprenorphine in a dosage from 2 mg to 16 mg. Table 3 below sets forth absorption data, including Cmax and AUC absorption levels, for the films having varying amounts of the active.

TABLE 2A
Absorption Data for Buprenorphine Films

| Buprenorphine | Cmax | AUCinf |
|---|---|---|
| 2 mg | 0.7-1.07 ng/ml | 6.8-9.5 hr · ng/ml |
| 4 mg | 1.2-1.84 ng/ml | 11.2-16.7 hr · ng/ml |
| 8 mg | 2.3-3.8 ng/ml | 22.7-34.1 hr · ng/ml |
| 12 mg | 2.8-5.2 ng/ml | 30.4-48.6 hr · ng/ml |
| 16 mg | 4.08-6.4 ng/ml | 42.6-65.8 hr · ng/ml |

### Comparative Example 3: Absorption Studies for Suboxone® Oral Tablets

Various tablet products were prepared and tested for absorption data, including Cmax, AUC, and Tmax absorption levels. The products tested included Suboxone® tablets made with 2, 4, 6, 8, 12 or 16 mg buprenorphine and 0.5, 1, 2, 3 or 4 mg naloxone. For 16 mg buprenorphine tablets, two 8 mg buprenorphine tablets were combined together to provide the level of components of a 16 mg buprenorphine tablet. In instances where a 12 mg buprenorphine tablet was evaluated, this dosage was obtained by combining one 8 mg buprenorphine tablet and two 2 mg buprenorphine tablets. These products were tested for absorption levels, with the amounts listed in Table 3 below.

TABLE 3
Absorption Data for Suboxone ® Tablets

| | | |
|---|---|---|
| Sample | Cmax | AUC |

| | | | | |
|---|---|---|---|---|
| Buprenorphine (2 mg) | 0.780 | ng/ml | 6.789 | hr * ng/ml |
| Suboxone ® Tablet | | | | |
| Naloxone (0.5 mg) | 51.30 | ng/ml | 128.60 | hr * ng/ml |
| Suboxone ® Tablet | | | | |
| Buprenorphine (16 mg) | 4.51 | ng/ml | 44.99 | hr * ng/ml |
| Suboxone ® Tablet | | | | |
| Naloxone (4 mg) | 259.00 | ng/ml | 649.60 | hr * ng/ml |
| Suboxone ® Tablet | | | | |
| Buprenorphine (4 mg) | 1.35 | ng/ml | 12.25 | hr * ng/ml |
| Suboxone ® Tablet | | | | |
| Naloxone (1 mg) | 80.97 | ng/ml | 203 | hr * ng/ml |
| Suboxone ® Tablet | | | | |
| Buprenorphine (8 mg) | 2.29 | ng/ml | 23.17 | hr * ng/ml |
| Suboxone ® Tablet | | | | |
| Naloxone (2 mg) | 140.31 | ng/ml | 351.8 | hr * ng/ml |
| Suboxone ® Tablet | | | | |
| Buprenorphine (12 mg) | 3.23 | ng/ml | 34.08 | hr * ng/ml |
| Suboxone ® Tablet | | | | |
| Naloxone (3 mg) | 199.7 | ng/ml | 500.6 | hr * ng/ml |
| Suboxone ® Tablet | | | | |

The absorption data for the self-supporting individual film dosage forms containing buprenorphine and naloxone was compared to the absorption data for the Buprenorphine/Naloxone Suboxone® Tablets. The results showed that about 30% less buprenorphine and naloxone was required in the film dosage unit than in the Suboxone® Tablet to achieve substantially equivalent Cmax, AUC, and Tmax values, i.e., within the range of about 80% to about 125%, at a confidence level of at least about 80%.

### Example 4: Comparison of Oral Products Containing Sildenafil

Self-supporting individual film dosage forms were prepared containing 22 mg, 45 mg, and 90 mg sildenafil, and the resulting films were tested for absorption data, including Cmax, AUC, and Tmax. The data was compared to the absorption data, including Cmax, A U C, and Tmax, of Viagra® oral tablets containing 25 mg, 50 mg and 100 mg of sildenafil. The results showed that 10% less sildenafil was required in the individual film dosage forms than in the oral tablet to achieve substantially equivalent Cmax, AUC, and Tmax values, i.e., within the range of about 80% to about 125%, at a confidence level of at least about 80%.

### Example 5: Comparison of Oral Products Containing Diazepam

Self-supporting individual film dosage forms were prepared containing 1 mg, 2.5 mg and 5 mg diazepam, and the resulting films were tested for absorption data, including Cmax, AUC, and Tmax. The data was compared to the absorption data, including Cmax, A U C, and Tmax, of Valium® oral tablets containing 2 mg, 5 mg and 10 mg of diazepam. The results showed that 50% less diazepam was required in the individual film dosage form than in the oral tablet to achieve substantially equivalent Cmax, AUC, and Tmax values, i.e., within the range of about 80% to about 125%, at a confidence level of at least about 80%.

### Example 6: Comparison of Oral Products Containing Rizatriptan

Self-supporting individual film dosage forms were prepared containing 2 mg and 4 mg rizatriptan, and the resulting films were tested for absorption data, including Cmax, AUC, and Tmax. The data was compared to the absorption data, including Cmax, A U C, and Tmax, of Maxalt® oral tablets containing 5 mg and 10 mg of rizatriptan. The results showed that 60% less rizatriptan was required in the individual film dosage form than in the oral tablet to achieve substantially equivalent Cmax, AUC, and Tmax values, i.e., within the range of about 80% to about 125%, at a confidence level of at least about 80%.

### Example 7: Comparison of Products Containing Epinephrine

Self-supporting individual film dosage forms were prepared containing 0.075 and 0.125 mg epinephrine, and the resulting films were tested for absorption data, including Cmax, AUC, and Tmax. The data was compared to the absorption data, including Cmax, A U C, and Tmax, for two epinephrine solutions of undiluted Adrenalin® for intramuscular or subcutaneous administration, containing 0.3 and 0.5 mg (0.3 mL and 0.5 mL, respectively) of epinephrine. The results showed that 75% less epinephrine was required in the individual film dosage form than in the epinephrine solution to achieve substantially equivalent Cmax, AUC, and Tmax values, i.e., within the range of about 80% to about 125%, at a confidence level of at least about 80%.

### Example 8: Comparison of Diazepam Film 20 mg Vs. Diastat® 20 mg Vagl Gel

Eleven human subjects were included in a pharmacokinetics study (subject 11 dosed period 1 but dismissed from study due to positive continine result and was not included in the analysis).

A Diazepam film 20 mg was made in accordance with the process of the present disclosure. Diazepam is a small molecule active. The Diazepam film 20 mg was administered to some of the human subjects via oral mucosal delivery and Diastat® 20 mg rectal gel (diazepam rectal gel) was administered to the remaining human subjects. Plasma concentrations were then measured. Test results are shown in Table 4.

TABLE 4
Test Results

| Pharmacokinetic Parameter | Geometric Mean | 90% Confidence |
|---|---|---|
| (PK Parameter)* | Ratio** (GMR %) | Interval (90% CI) |
| AUCt | 127.40 | 83.70-193.89 |
| AUCinf | 136.88 | 80.54-232.70 |
| Cmax | 182.06 | 127.31-260.36 |
| Tmax | Median | Range |
| Diazepam Film 20 mg | 1.50 h | 0.36-2.05 h |
| Diastat ® 20 mg rectal gel | 1.0 h | 0.15-2.00 h |

*Diastat ® 20 mg rectal gel pharmacokinetic were assessed without an assessment of degree of bowel emptying.
**Ratio of film to gel.

As is apparent from Table 4, the AUCt and AUCinf 90% CI range were greater than the upper limit of 125%. Cmax was also entirely above the upper limit of 125%.

As is also apparent from Table 4, although the median Tmax for Diazepam Film 20 mg was 0.5 hours slower than for Diastat® 20 mg rectal gel, the Tmax ranges for Diazepam Film 20 mg and for Diastat® 20 mg rectal gel were comparable.

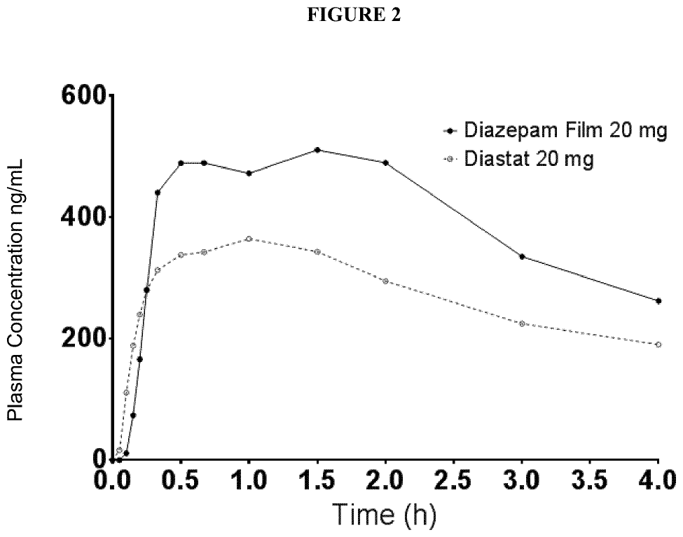

As shown in FIG. 2, the 20 mg Diazepam Film showed improved bioavailability and Cmax relative to Diastat® 20 mg rectal gel.

As evidenced by this example using transmucosal delivery can affect the pharmacokinetic parameters differently. The film delivered a total greater amount of drug.

### Example 9: Comparison of 15 mg Diazepam Buccal Film to 20 mg Diastat® Rectal Gel in Fasting and Fed States

A 15 mg Diazepam film was made in accordance with the process of the present disclosure.

A study was conducted in which the 15 mg Diazepam film was administered to the buccal mucosa of human subjects who were in a fasted state at the time of administration and to the buccal mucosa of human subjects who were in a fed state at the time of administration.

Table 5 shows ratios (B/A), 90% geometric confidence intervals, intra- and inter-subjects CV (%) for the 15 mg Diazepam buccal film.

TABLE 5
Ratios (B/A), 90% Geometric Confidence Intervals,
Intra- and Inter-Subjects CV (%) for Diazepam

| | | | | Intra- | Inter- |
|---|---|---|---|---|---|
| Pharmacokinetic | 90% Geometric C.I.² | | | Subject | Subject |
| parameters | Ratio^{1 3} | Lower | Upper | CV | CV |
| AUC 0-t | 98.84% | 93.18% | 104.85% | 10.16% | 33.38% |
| AUC 0-inf | 98.84% | 92.07% | 105.68% | 11.88% | 38.33% |
| Cmax | 55.16% | 48.55% | 62.67% | 22.20% | 5.53% |

¹Calculated using least-squares means according to the formula: e^{(DIFFERENCE)} × 100.
²90% Geometric Confidence Interval using In-transformed data
³Ratio of B to A where B = fed state and A = fasted state.

As is apparent from table 5, the Cmax was changed in the fed state. In particular, the Cmax was reduced by approximately 45% in the fed state. It was particularly surprising that the oral dosage only has a Cmax reduction of approximately 28%. The transmucosal delivery has a significant impact on the Cmax.

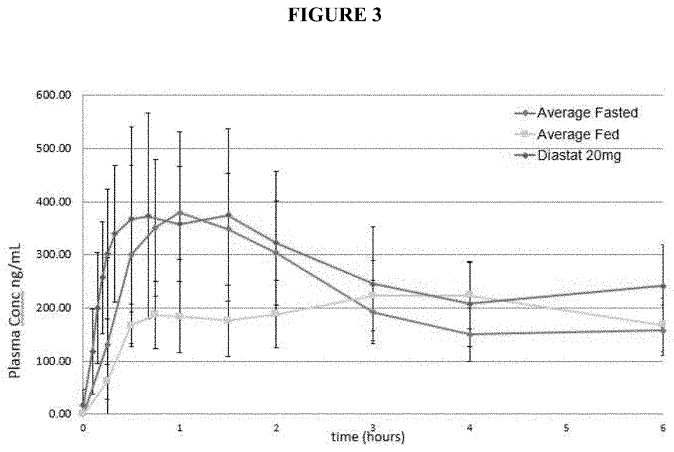

As is shown in FIG. 3, the 15 mg Diazepam buccal film exhibited bioavailability that was substantially equivalent to that of the 20 mg Diastat® rectal gel when administered to human subjects in a fasted state but that was significantly less when administered to human subjects in a fed state. The 15 mg Diazepam buccal film thus showed the ability to be both sub and super potent.

### Example 10: Comparison of 24 mg Dipivefrin Film Administered Transmucosally and Orally (Swallowed)

A 24 mg Dipivefrin film was made in accordance with the process of the present disclosure.

A study was conducted in which the 24 mg Dipivefrin film was administered to some human subjects bysublingul administration. Other human subjects swallowed a 24 mg Dipivefrin film with 240 mL water (enteral administration).

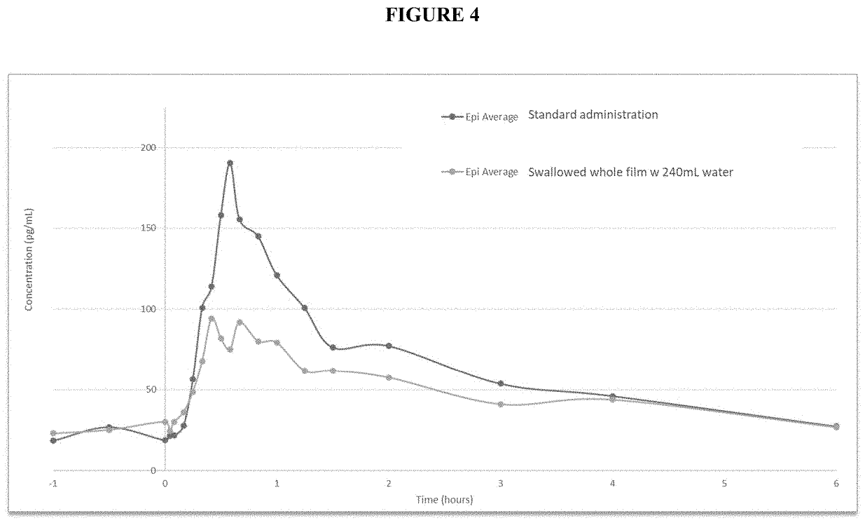

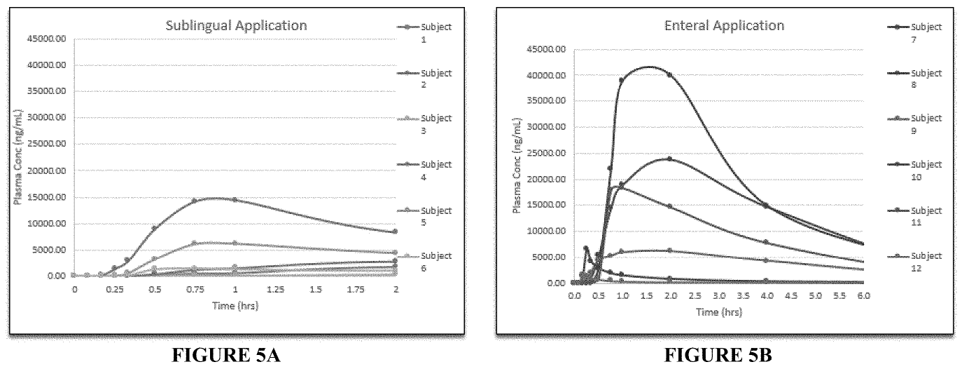

Bioavailability results from the study are shown in FIG. 4. As is apparent from FIG. 4, transmucosal delivery provided enhanced bioavailability and Cmax in comparison to oral absorption.

### Example 11: Comparison of 10 mg Octreotide Film Administered Transmucosally and Orally (Enterally)

A 10 mg Octreotide film was made in accordance with the process of the present disclosure.

A study was conducted to compare the effect of administrating the Octreotide 10 mg film transmucosally (by sublingual application) and orally (enteral application) to human subjects.

Total AUC for the orally administered film (enteral application) and for the transmucosally administered film is shown in Table 8.

TABLE 8
Total AUC

| Route of | Individual AUC's | | | | | | |
|---|---|---|---|---|---|---|---|
| Administration | (ng/ml/hr) | | | | | | Total AUC |
| Oral | 107898 | 152205 | 33770 | 5687 | 68285 | 2322 | 61694.5 |
| (Enteral) | | | | | | | |
| Transmucosal | 8519 | 42208 | 4609 | 14708 | 21172 | 9488 | 16784 |

As shown by the Total AUC values in Table 8, total AUC increased by 368% when the film was administered orally (via enteral application) versus transmucosally.

While there have been described what are presently believed to be the certain desirable embodiments of the invention, those skilled in the art will realize that changes and modifications may be made thereto without departing from the spirit of the invention, and it is intended to include all such changes and modifications as fall within the true scope of the invention.

1. A self-supporting film dosage form for oral mucosal delivery of a pharmaceutical active in individual dosage units comprising a water-soluble or water swellable film-forming polymer matrix containing a pharmaceutical active,
wherein when said film dosage form is administered to the oral mucosa of a mammal, a pharmacokinetic profile of the pharmaceutical active is substantially equivalent to the pharmacokinetic profile of a corresponding enterally delivered dosage form containing the pharmaceutical active, and
wherein the film dosage form for mucosal delivery contains about 0.5% to about 75% less active per individual dosage unit than the corresponding enterally delivered dosage form.

2. The self-supporting film dosage form of claim 1, wherein one or more of the AUC and Cmax of the film dosage form is about 80% to about 125% at a confidence level of at least about 80%, of the AUC and/or Cmax of the corresponding enterally delivered dosage form.

3. The self-supporting film dosage form of claim 1, wherein the corresponding enterally delivered dosage form is selected from the group consisting of tablets, capsules, lozenges, troches, wafers, caplets, chewables, gels, liquids, orally dissolving tablets (ODTs), lyophilized dosage forms and any combination thereof.

4. The self-supporting film dosage form of claim 1, wherein the pharmacokinetic profile of the film dosage form (pKP_{film}) is about 90% to about 115% of the pharmacokinetic profile of the corresponding enterally delivered dosage form (pKP_{ingestible}) at a confidence level of at least about 70%.

5. The self-supporting film dosage form of claim 1, wherein the pharmacokinetic profile of the film dosage form (pKP_{film}) is about 95% to about 110% of the pharmacokinetic profile of the corresponding enterally delivered dosage form (pKP_{ingestible}) at a confidence level of at least about 60%.

6. The self-supporting film dosage form of claim 1, wherein the Tmax is about 80% to about 125% of the corresponding enterally delivered dosage form.

7. The self-supporting film dosage form of claim 1, wherein the pharmaceutical active is a pharmaceutically acceptable small molecule.

8. The self-supporting film dosage form of claim 7, wherein the pharmaceutical active is a prodrug, derivative, analogue or any combination thereof.

9. The self-supporting film dosage form of claim 1, further including a penetration enhancer.

10. The self-supporting film dosage form of claim 9, wherein the penetration enhancer is a phytoextract or a phenylpropanoid.

11.-13. (canceled)

14. The self-supporting film dosage form of claim 1, wherein the water-soluble or water swellable film-forming polymer matrix comprises a polymer selected from the group consisting of polyethylene oxide, pullulan, hydroxypropylmethyl cellulose, hydroxyethyl cellulose, hydroxyethylmethyl cellulose, hydroxypropyl cellulose, polyvinyl pyrrolidone, methylcellulose, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, polysaccharides, and any combinations thereof.

15. The self-supporting film dosage form of claim 1, wherein the water-soluble or water swellable film-forming polymer matrix comprises at least one polymer selected from the group consisting of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

16. (canceled)

17. The self-supporting film dosage form of claim 1, wherein the pharmaceutical active is selected from the following therapeutic classes: ace-inhibitor; adrenergic agent; adrenocortical steroid; adrenocortical suppressant; aldosterone antagonist; alkaloid; amino acid; anabolic; analeptic; analgesic; anesthetic; anorectic; anti-acne agent; anti-adrenergic; anti-allergic; anti-amebic; anti-anemic; anti-anginal; anti-anxiety; anti-arthritic; anti-arrythmia; anti-asthmatic; anti-atherosclerotic; anti-cholesterolemic; antibacterial; antibiotic; anticholinergic; anticoagulant; anticonvulsant; antidepressant; antidiabetic; antidiarrheal; antidiuretic; antidote; anti-emetic; anti-epileptic; antifibrinolytic; antifungal; antihemorrhagic; antihistamine; antihyperlipidemia; antihypertensive; antihypotensive; anti-infective (both systemic and non-systemic); anti-inflammatory; anti-lipid; anti-manic; antimicrobial; antimigraine; antimitotic; antimycotic, antinauseant; antineoplastic; antineutropenic; anti-obesity; antiparasitic; anti-parkinson; antiproliferative; antipsychotic; anti-pyretic; antirheumatic; antiseborrheic; antisecretory; antispasmodic; anti-stroke; antithrombotic; anti-thyroid; anti-tumor; anti-tussive; anti-ulcerative; anti-uricemic; antiviral; appetite suppressant; appetite stimulant; biological response modifier; blood glucose regulator; blood modifier; blood metabolism regulator; bone resorption inhibitor; bronchodilator; cardiovascular agent; central nervous system stimulant; cerebral dilator; contraceptive; coronary dilator; cholinergic; cough suppressant; decongestant; depressant; diagnostic aid; dietary supplement; diuretic; dopaminergic agent; enzymes; estrogen receptor agonist; endometriosis management agent; expectorant; erectile dysfunction therapy; erythropoietic; ibrinolytic; fertility agent; fluorescent agent; free oxygen radical scavenger; gastric acid suppressant; gastrointestinal motility effector; genetic modifier; glucocorticoid; hair growth stimulant; hemostatic; histamine H2 receptor antagonists; homeopathic remedy; hormone; hypercalcemia management agent; hypocalcemia management agent; hypocholesterolemic; hypoglycemic; hypolipidemic; hypotensive; ion exchange resin; imaging agent; immunizing agent; immunomodulator; immunoregulator; immunostimulant; immunosuppressant; keratolytic; laxative; LHRH agonist; mood regulator; motion sickness preparation; mucolytic; muscle relaxant; mydriatic; nasal decongestant; neuromuscular blocking agent; neuroprotective; NMDA antagonist; non-hormonal sterol derivative; osteoporosis therapy; oxytocic; parasympatholytic; parasympathomimetic; plasminogen activator; platelet activating factor antagonist; platelet aggregation inhibitor; prostaglandin; psychotherapeutic; psychotropic; radioactive agent; respiratory agent; scabicide; sclerosing agent; sedative; sedative-hypnotic; selective adenosine A1 antagonist; serotonin antagonist; serotonin inhibitor; serotonin receptor antagonist; smoking cessation therapy; steroid; stimulant; sympatholytic; terine relaxant; thyroid hormone; thyroid inhibitor; thyromimetic; tranquilizer; tremor therapy; amyotrophic lateral sclerosis agent; cerebral ischemia agent; Paget's disease agent; unstable angina agent; vasoconstrictor; vasodilator; weight management; wound healing agent; xanthine oxidase inhibitor; and combinations thereof.

18. The self-supporting film dosage form of claim 1, wherein the pharmaceutical active is selected from the group consisting of rizatriptan, testosterone, diazepam, pregabalin (Lyrica), epinephrine, sildenafil, clobazam, riluzole, apomorphine, buprenorphine, naloxone and lumateperone.

19. The self-supporting film dosage form of claim 1, further comprising an NSAID.

20.-34. (canceled)

35. A pharmaceutically acceptable film for oral mucosal delivery of a pharmaceutical active comprising:
a. a pharmaceutical active; and
b. a water-soluble or water swellable film-forming polymer matrix comprising
i. a polyethylene oxide composition,
ii. water or a water-containing solvent,
iii. and silicon dioxide;
wherein the oral film is an individual unit dosage form,
wherein, when the individual unit dosage form is administered to the oral mucosa of a patient, one or more of the AUC and Cmax of the pharmaceutical active is about 80% to about 125% at a confidence level of at least about 80% of the AUC and/or Cmax of the corresponding enterally delivered dosage form, and
wherein each individual unit dosage form contains about 0.5% to about 50% less active than the corresponding enterally delivered dosage form.

36. The pharmaceutically acceptable film of claim 35, wherein the polyethylene oxide composition includes the silicon dioxide in about 0.02% to about 3% by weight of the polymer matrix.

37. The pharmaceutically acceptable film of claim 35, wherein the Tmax is about 80% to about 125% of the corresponding enterally delivered dosage form.

38. A method of treating a patient comprising administering the self-supporting film dosage form of claim 1 to a patient in need thereof.

39. A method of treating a patient comprising administering the film of claim 35 to a patient in need thereof.

40.-71. (canceled)
